(19) United States
(12) Patent Application Publication
(10) Pub. No.: US 2022/0025462 A1
(43) Pub. Date: Jan. 27, 2022

(54) NUCLEIC ACID BIOMARKERS FOR PLACENTAL DYSFUNCTION

(71) Applicants: Sera Prognostics, Inc., Salt Lake City, CA (US); THE REGENTS OF THE UNIVERSITY OF CALIFORNIA, Oakland, CA (US)

(72) Inventors: John Jay Boniface, Salt Lake City, UT (US); Julja Burchard, Holladay, UT (US); Ryan Michael Treacy, Salt Lake City, UT (US); Louise C. Laurent, La Jolla, CA (US)

(21) Appl. No.: 17/312,326

(22) PCT Filed: Dec. 9, 2019
(86) PCT No.: PCT/US19/65277

§ 371 Date: Jun. 9, 2021

Related U.S. Application Data
(60) Provisional application No. 62/777,576, filed on Dec. 10, 2018.

(51) Int. Cl. C12Q 1/6883

(52) U.S. Cl. CPC C12Q 1/6883; C12Q 2600/178

(57) ABSTRACT

The present invention provides compositions and methods for determining a pregnant female's risk of developing placental dysfunction later in the pregnancy.

This application claims the benefit of U.S. Provisional application No. 62/777,576 filed Dec. 10, 2018, the entire contents of which are incorporated herein by reference.

### BACKGROUND

Placental dysfunction, most commonly manifested as preeclampsia or intrauterine growth restriction, is an important cause of maternal and fetal morbidity and mortality in both the developing and developed world. placental insufficiency. In particular, placental dysfunction linked to preterm birth (PTB), preeclampsia, intrauterine growth restriction, preterm labor, preterm premature rupture of membranes, late spontaneous abortion and abruption placentae. It is thought that placental dysfunction arises from abnormal trophoblast differentiation and/or invasion, events that occur in the first trimester of pregnancy, but become clinically apparent only in the late second and third trimesters. Optimal surveillance and management of placental dysfunction, as well as the development of effective therapies, have been hampered by the lack of methods for early and accurate identification of pregnancies at risk for this disorder. MicroRNAs (miRNAs) are non-coding, 21-25 nucleotide, regulatory RNAs that affect the stability and/or translational efficiency of messenger-RNA (mRNAs). There is a need to develop a maternal blood-based assay for quantification of extracellular microRNA (miRNA) biomarkers present in the maternal serum during the second trimester of pregnancy in order to determine a pregnant female's risk of developing placental dysfunction later in the pregnancy. The present invention addresses this need and provides related advantages.

### SUMMARY

The present invention provides compositions and methods for determining a pregnant female's risk of developing placental dysfunction later in the pregnancy.

In one aspect, the invention provides a panel of isolated nucleic acid biomarkers comprising two or more of the nucleic acid biomarkers set forth in Tables 3-11 or 15-18.

In a further aspect, the invention provides a composition of labeled and/or amplified nucleic acid molecules, for example, amplified labeled nucleic acid molecules, that correspond to two or more of the nucleic acid biomarkers set forth in Tables 3-11 or 15-18.

In a further aspect, the invention provides a panel of isolated nucleic acid biomarkers comprising two or more of the nucleic acid biomarkers set forth in Tables 3-6, 15, 17 or 18.

In an additional aspect, the invention provides a composition of labeled and/or amplified nucleic acid molecules, for example, amplified labeled nucleic acid molecules, that correspond to two or more of the nucleic acid biomarkers set forth in Tables 3-6, 15, 17 or 18.

In additional embodiments, the invention provides a biomarker panel comprising two or more of the isolated nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941.

In some embodiments, the biomarker panel comprises isolated nucleic acid biomarkers comprising hsa-miR-331-3p and/or hsa-miR-941. In some embodiments, the biomarker panel comprises isolated nucleic acid biomarkers comprising hsa-miR-423-3p, hsa-miR-516b-5p, hsa-miR-4732-5p, and/or hsa-miR-1273h-3p. In some embodiments, the biomarker panel comprises isolated nucleic acid biomarkers comprising hsa-miR-4732-5p, hsa-miR-516b-5p, and/or hsa-miR-941. In some embodiments, the biomarker panel comprises isolated nucleic acid biomarkers comprising hsa-miR-155-5p, hsa-miR-331-3p, hsa-miR-1273h-3p, and/or hsa-miR-516b-5p.

In a further aspect, the invention provides a composition of labeled and/or amplified nucleic acid molecules, for example, amplified labeled nucleic acid molecules, that correspond to two or more of the nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941.

In an additional aspect, the invention provides a composition of labeled and/or amplified nucleic acid molecules, for example, amplified labeled nucleic acid molecules, that correspond to nucleic acid biomarkers hsa-miR-331-3p and/or hsa-miR-941. In an additional aspect, the invention provides a composition of labeled and/or amplified nucleic acid molecules, for example, amplified labeled nucleic acid molecules, that correspond to nucleic acid biomarkers hsa-miR-423-3p, hsa-miR-516b-5p, hsa-miR-4732-5p, and/or hsa-miR-1273h-3p. In an additional aspect, the invention provides a composition of labeled and/or amplified nucleic acid molecules, for example, amplified labeled nucleic acid molecules, that correspond to nucleic acid biomarkers hsa-miR-4732-5p, hsa-miR-516b-5p, and/or hsa-miR-941. In an additional aspect, the invention provides a composition of labeled and/or amplified nucleic acid molecules, for example, amplified labeled nucleic acid molecules, that correspond to nucleic acid biomarkers hsa-miR-155-5p, hsa-miR-331-3p, hsa-miR-1273h-3p, and/or hsa-miR-516b-5p.

In one aspect, the invention provides a pair of biomarker selected from the group consisting of the nucleic acid biomarkers set forth in Tables 3-11 or 15-18.

In a further aspect, the invention provides a composition of labeled and/or amplified nucleic acid molecules, for example, amplified labeled nucleic acid molecules, that correspond to a pair of biomarker selected from the group consisting of the nucleic acid biomarkers set forth in Tables 3-11 or 15-18.

In one aspect, the invention provides a panel of isolated nucleic acid biomarkers comprising a pair of biomarkers selected from the group consisting of the biomarker pairs set forth in Tables 7-10 or 16-18.

In further embodiments, the invention provides a pair of nucleic acid biomarkers selected from the group consisting of the biomarker pairs set forth in Tables 7-10 or 16-18.

In a further aspect, the invention provides a composition of labeled and/or amplified nucleic acid molecules, for example, amplified labeled nucleic acid molecules, that correspond to a pair of biomarker selected from the group consisting of the biomarker pairs set forth in Tables 7-10 or 16-18.

In further embodiments, the invention provides a pair of nucleic acid biomarkers selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

In further aspect, the invention provides a panel of isolated nucleic acid biomarkers comprising a pair of biomarkers selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

In additional embodiments of the pair of biomarkers or panel of biomarkers comprising a pair of biomarkers, the pair of biomarkers is selected from the group consisting of hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p.

In additional embodiments of the pair of biomarkers or panel of biomarkers comprising a pair of biomarkers, the pair of biomarkers is selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p.

In additional embodiments of the pair of biomarkers or panel of biomarkers comprising a pair of biomarkers, the pair of biomarkers is selected from the group consisting of hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c.

In additional embodiments of the pair of biomarkers or panel of biomarkers comprising a pair of biomarkers, the pair of biomarkers is selected from the group consisting of hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

In a further aspect, the invention provides a composition of labeled and/or amplified nucleic acid molecules, for example, amplified labeled nucleic acid molecules, wherein the labeled and/or amplified nucleic acid molecules, for example, amplified labeled nucleic acid molecules, correspond to a pair of biomarkers selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

In some embodiments, the invention provides a composition of labeled and/or amplified nucleic acid molecules, for example, amplified labeled nucleic acid molecules, wherein the labeled and/or amplified nucleic acid molecules, for example, amplified labeled nucleic acid molecules, correspond to a pair of biomarkers selected from the group consisting of hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p.

In some embodiments, the invention provides a composition of labeled and/or amplified nucleic acid molecules, for example, amplified labeled nucleic acid molecules, wherein the labeled and/or amplified nucleic acid molecules, for example, amplified labeled nucleic acid molecules, correspond to a pair of biomarkers selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p.

In some embodiments, the invention provides a composition of labeled and/or amplified nucleic acid molecules, for example, amplified labeled nucleic acid molecules, wherein the labeled and/or amplified nucleic acid molecules, for example, amplified labeled nucleic acid molecules, correspond to a pair of biomarkers selected from the group consisting of hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c.

In some embodiments, the invention provides a composition of labeled and/or amplified nucleic acid molecules, for example, amplified labeled nucleic acid molecules, wherein the labeled and/or amplified nucleic acid molecules, for example, amplified labeled nucleic acid molecules, correspond to a pair of biomarkers selected from the group consisting of hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

Also provided by the invention is a method of determining a pregnant female's risk of developing placental dysfunction later in the pregnancy comprising measuring the amount of two or more of the nucleic acid biomarkers listed in Tables 3-11 or 15-18 in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to two or more of the nucleic acid biomarkers set forth in Tables 3-11 or 15-18 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

Also provided by the invention is a method of determining a pregnant female's risk of developing placental dysfunction later in the pregnancy comprising measuring the amount of two or more of the nucleic acid biomarkers listed in Tables 3-6, 15, 17 or 18 in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to two or more of the nucleic acid biomarkers set forth in Tables 3-6, 15, 17 or 18 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

Also provided by the invention is a method of determining a pregnant female's risk of developing placental dysfunction later in the pregnancy comprising measuring the amount of two or more of the nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941 in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to two or more of the nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

Also provided by the invention is a method of determining a pregnant female's risk of developing placental dysfunction later in the pregnancy comprising measuring the amount of hsa-miR-331-3p and/or hsa-miR-941 in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-331-3p and/or hsa-miR-941 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

Also provided by the invention is a method of determining a pregnant female's risk of developing placental dysfunction later in the pregnancy comprising measuring the amount of hsa-miR-423-3p, hsa-miR-516b-5p, hsa-miR-4732-5p, and/or hsa-miR-1273h-3p in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-423-3p, hsa-miR-516b-5p, hsa-miR-4732-5p, and/or hsa-miR-1273h-3p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

Also provided by the invention is a method of determining a pregnant female's risk of developing placental dysfunction later in the pregnancy comprising measuring the amount of hsa-miR-4732-5p, hsa-miR-516b-5p, and/or hsa-miR-941 in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-4732-5p, hsa-miR-516b-5p, and/or hsa-miR-941 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

Also provided by the invention is a method of determining a pregnant female's risk of developing placental dysfunction later in the pregnancy comprising measuring the amount of hsa-miR-155-5p, hsa-miR-331-3p, hsa-miR-1273h-3p, and/or hsa-miR-516b-5p in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-155-5p, hsa-miR-331-3p, hsa-miR-1273h-3p, and/or hsa-miR-516b-5p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising measuring the amount of a pair of nucleic acid biomarkers selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

In some embodiments, the invention provides a method of determining a pregnant female's risk of developing placental dysfunction later in the pregnancy comprising measuring the amount of a pair of nucleic acid biomarkers selected from the group consisting of hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

In further embodiments, the invention provides a method of determining a pregnant female's risk of developing placental dysfunction later in the pregnancy comprising measuring the amount of a pair of nucleic acid biomarkers selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

In further embodiments, the invention provides a method of determining a pregnant female's risk of developing placental dysfunction later in the pregnancy comprising measuring the amount of a pair of nucleic acid biomarkers selected from the group consisting of hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

In further embodiments, the invention provides a method of determining a pregnant female's risk of developing placental dysfunction later in the pregnancy comprising measuring the amount of a pair of nucleic acid biomarkers selected from the group consisting of hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p in a biological sample obtained from the pregnant female, and calculating a risk score based upon the measured amounts of the nucleic acid biomarkers to determine the pregnant female's risk of developing placental dysfunction. In some embodiments, the method further comprises the step of providing a score corresponding to the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the method further comprises the step of providing a score corresponding to the pregnant female's risk of developing placental dysfunction.

In some embodiments of methods of the invention, the risk score is calculated based on a ratio of data values. In some embodiments of methods of the invention, data transformation is applied before or after the ratio is determined.

Other features and advantages of the invention will be apparent from the detailed description, and from the claims.

### BRIEF DESCRIPTION OF THE DRAWINGS

FIGS. 1A and 1B show Principle Component Analysis plots. Principle Component Analysis for the extracellular miRNA data (all possible reversals) using the log values of the ratios (FIG. 1A) or the ratios of log values (FIG. 1B) as the features.

### DETAILED DESCRIPTION

The present disclosure is based, generally, on the discovery that the concentration of certain extracellular microRNA (miRNA) biomarkers present in the maternal circulation during pregnancy predicts subsequent risk of developing placental dysfunction later in the pregnancy. For each of the miRNA biomarkers disclosed herein, the concentration of miRNA in the maternal circulation is altered in women who subsequently develop placental dysfunction. Advantageously, expression levels of these miRNA biomarkers can be measured from blood samples, thereby providing a minimally-invasive means for prediction of placental dysfunction, which can manifest as preeclampsia, intrauterine growth restriction, preterm birth (PTB), preterm labor, preterm premature rupture of membranes, late spontaneous abortion and abruption placentae.

The present disclosure is further specifically based, in part, on the unexpected discovery that single-miRNA biomarker and pairs of miRNA biomarkers disclosed herein can be utilized in methods of predicting a pregnant female's risk of developing placental dysfunction later in the pregnancy. Each of the miRNA biomarkers and clinical variables disclosed herein, either alone or as components of pairs, ratios and/or reversal pairs serve as biomarkers for determining a pregnant women's risk of developing placental dysfunction later in the pregnancy.

A reversal value is the ratio of the abundance of an up regulated biomarker over a down regulated biomarker and serves to both normalize variability and amplify diagnostic signal. The invention lies, in part, in the selection of particular biomarkers that, when paired together, can accurately determine a pregnant female's risk of developing placental dysfunction later in the pregnancy. Accordingly, it is human ingenuity in selecting the specific biomarkers that are informative upon being paired, for example, in novel reversals, and/or the data transformations, for example the ratio of log values, in forming said reversals, that underlies the present invention.

The disclosure provides single-miRNA biomarkers and pairs of miRNA biomarkers as well as associated panels, methods and kits for determining a pregnant female's risk of developing placental dysfunction later in the pregnancy.

In addition to the specific miRNA biomarkers identified in this disclosure, for example, by name, sequence, or reference, the invention also contemplates use of biomarker variants that are at least 90% or at least 95% or at least 97% identical to the exemplified sequences and that are now known or later discovered and that have utility for the methods of the invention. These variants may represent polymorphisms, splice variants, mutations, and the like. In this regard, the instant specification discloses multiple art-known proteins in the context of the invention and provides exemplary peptide sequences that can be used to identify these proteins. However, those skilled in the art appreciate that additional sequences or other information can easily be identified that can provide additional characteristics of the disclosed biomarkers and that the exemplified references are in no way limiting with regard to the disclosed nucleic acid.

As described herein, various techniques and reagents find use in the methods of the present invention. Suitable samples in the context of the present invention include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. As described herein, nucleic acid can be detected through a variety of assays and techniques known in the art.

The miRNA biomarkers that can be components of reversal pairs described herein include, for example, the miRNA biomarkers set forth in Tables 7-10 or 16-18.

In some embodiments, the invention provides a method of determining a pregnant female's risk of developing placental dysfunction later in the pregnancy, the method comprising measuring in a biological sample obtained from the pregnant female a reversal value for one or more of the biomarker pairs set forth in Tables 7-10 or 16-18.

In some embodiments, the invention provides a method of determining a pregnant female's risk of developing placental dysfunction later in the pregnancy, the method comprising measuring in a biological sample obtained from the pregnant female a reversal value for one pair of biomarkers selected from the biomarker pairs set forth in Tables 7-10 or 16-18.

In some embodiments, the invention provides a pair of isolated biomarkers selected from the biomarker pairs set forth in Tables 7-10 or 16-18, wherein the pair of biomarkers exhibits a higher ratio in pregnant females that will develop placental dysfunction later in the pregnancy relative to pregnant females that will not develop placental dysfunction.

In one embodiment, the present invention provides a composition comprising a pair of isolated biomarkers selected from the group consisting of the biomarker pairs listed in Tables 7-10 or 16-18, wherein the pair of biomarkers exhibits a higher ratio in pregnant females that will develop placental dysfunction later in the pregnancy relative to pregnant females that will not develop placental dysfunction.

In some embodiments, the sample is obtained between 18 and 21 weeks of GABD. In further embodiments, the sample is obtained between 23 and 28 weeks of GABD. In some embodiments, the sample is obtained between 18 and 28 weeks of GABD. In some embodiments, the sample is obtained between 18 and 36 weeks of GABD. In further embodiments the sample is obtained between 19 and 21 weeks of GABD. In some embodiments, the sample is obtained between 20 and 22 weeks of GABD. In some embodiments, the sample is obtained between 21 and 23 weeks of GABD. In further embodiments, the sample is obtained between 22 and 24 weeks of GABD. In additional embodiments, the sample is obtained between 23 and 25 weeks of GABD. In some embodiments, the sample is obtained between 24 and 26 weeks of GABD. In further embodiments, the sample is obtained between 25 and 27 weeks of GABD. In additional embodiments, the sample is obtained between 26 and 28 weeks of GABD. In some embodiments, the sample is obtained between 27 and 29 weeks of GABD. In further embodiments, the sample is obtained between 28 and 30 weeks of GABD. In additional embodiments, the sample is obtained between 29 and 31 weeks of GABD. In some embodiments, the sample is obtained between 30 and 32 weeks of GABD. In further embodiments, the sample is obtained between 31 and 33 weeks of GABD. In additional embodiments, the sample is obtained between 32 and 34 weeks of GABD. In some embodiments, the sample is obtained between 33 and 35 weeks of GABD. In further embodiments, the sample is obtained between 34 and 36 weeks of GABD. In additional embodiments, the sample is obtained between 18 and 21 weeks of GABD.

In some embodiments, the sample is obtained between 119 and 202 days of GABD. In further embodiments, the sample is obtained between 119 and 152 days of GABD. In some embodiments, the sample is obtained between 138 and 172 days of GABD. In further embodiments, the sample is obtained between 156 and 196 days of GABD.

In addition to the specific biomarkers, the disclosure further includes biomarker variants that are about 90%, about 95%, or about 97% identical to the exemplified sequences. Variants, as used herein, include polymorphisms, splice variants, mutations, and the like. Although described with reference to protein biomarkers, changes in reversal value can be identified in protein or gene expression levels for pairs of biomarkers.

The compositions and methods of the invention also can include clinical variables, including but not limited to, maternal characteristics, medical history, past pregnancy history, and obstetrical history. Such additional clinical variables can include, for example, previous low birth weight or preterm delivery, multiple 2nd trimester spontaneous abortions, prior first trimester induced abortion, familial and intergenerational factors, history of infertility, parity, nulliparity, placental abnormalities, cervical and uterine anomalies, short cervical length measurements, gestational bleeding, intrauterine growth restriction, in utero diethylstilbestrol exposure, multiple gestations, infant sex, short stature, low prepregnancy weight, low or high body mass index, diabetes, diabetes mellitus, chronic diabetes, chronic diabetes mellitus, chronic hypertension, urogenital infections (i.e. urinary tract infection), asthma, anxiety and depression, asthma, hypertension, hypothyroidism, high body mass index (BMI), low BMI, BMI. Demographic risk indicia for preterm birth can include, for example, maternal age, race/ethnicity, single marital status, low socioeconomic status, maternal age, employment-related physical activity, occupational exposures and environment exposures and stress. Further clinical variables can include, inadequate prenatal care, cigarette smoking, use of marijuana and other illicit drugs, cocaine use, alcohol consumption, caffeine intake, maternal weight gain, dietary intake, sexual activity during late pregnancy and leisure-time physical activities. (Preterm Birth: Causes, Consequences, and Prevention, Institute of Medicine (US) Committee on Understanding Premature Birth and Assuring Healthy Outcomes; Behrman R E, Butler A S, editors. Washington (D.C.): National Academies Press (US); 2007). Additional clinical variables useful for as markers can be identified using learning algorithms known in the art, such as linear discriminant analysis, support vector machine classification, recursive feature elimination, prediction analysis of microarray, logistic regression, CART, FlexTree, LART, random forest, MART, and/or survival analysis regression, which are known to those of skill in the art and are further described herein.

The present disclosure describes and exemplifies various models and corresponding biomarkers that perform at high levels of accuracy and precision in determining a pregnant female's risk of developing placental dysfunction later in the pregnancy

It will be understood by those of skill in the art, that other models are known in the art that can be used to practice the claimed inventions and that the performance of a model can be evaluated in a variety of ways, including, but not limited to accuracy, precision, recall/sensitivity, weighted average of precision and recall. Models known in the art include, without limitation, linear discriminant analysis, support vector machine classification, recursive feature elimination, prediction analysis of microarray, logistic regression, CART, FlexTree, LART, random forest, MART, and/or survival analysis regression.

In some embodiments, performance of a model can be evaluated based on accuracy. For example, accuracy can be expressed as the percentage of time, for example, 50%, 51%, 52%, 53%, 54%, 55%, 56%, 57%, 58%, 59%, 60%, 61%, 62%, 63%, 64%, 65%, 70%, 71%, 72%, 73%, 74%, 75%, 76%, 77%, 80% or more that a model accurately predicts a pregnant female's risk of developing placental dysfunction later in the pregnancy.

The present disclosure is based in part on the surprising discovery that the selection of certain biomarkers and/or clinical variables enables determining a pregnant female's risk of developing placental dysfunction later in the pregnancy.

It must be noted that, as used in this specification and the appended claims, the singular forms “a”, “an” and “the” include plural referents unless the content clearly dictates otherwise. Thus, for example, reference to “a biomarker” includes a mixture of two or more biomarkers, and the like.

The term “about,” particularly in reference to a given quantity, is meant to encompass deviations of plus or minus five percent.

As used in this application, including the appended claims, the singular forms “a,” “an,” and “the” include plural references, unless the content clearly dictates otherwise, and are used interchangeably with “at least one” and “one or more.”

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

As used herein, the term “panel” refers to a composition, such as an array or a collection, comprising one or more biomarkers. The term can also refer to a profile or index of expression patterns of one or more biomarkers described herein. The number of biomarkers useful for a biomarker panel is based on the sensitivity and specificity value for the particular combination of biomarker values.

As used herein, and unless otherwise specified, the terms “isolated” and “purified” generally describes a composition of matter that has been removed from its native environment (e.g., the natural environment if it is naturally occurring), and thus is altered by the hand of man from its natural state so as to possess markedly different characteristics with regard to at least one of structure, function and properties. An isolated protein or nucleic acid is distinct from the way it exists in nature and includes synthetic peptides and proteins.

The term “biomarker” refers to a biological molecule, a fragment of a biological molecule, or a clinical variable the change and/or the detection of which can be correlated with a particular physical condition or state. The terms “marker” and “biomarker” are used interchangeably throughout the disclosure. For example, the biomarkers of the present invention are associated with a discrimination power between pregnant females that will develop placental dysfunction later in the pregnancy versus those that will not develop placental dysfunction. Such biomarkers include any suitable analyte, but are not limited to, biological molecules comprising nucleotides, nucleic acids, nucleosides, amino acids, sugars, fatty acids, steroids, metabolites, peptides, polypeptides, proteins, carbohydrates, lipids, hormones, antibodies, regions of interest that serve as surrogates for biological macromolecules and combinations thereof (e.g., glycoproteins, ribonucleoproteins, lipoproteins). The term also encompasses miRNAs and portions or fragments of a miRNAs.

As used herein, the term “reversal” refers to the ratio of the measured abundance of an upregulated analyte over that of a down-regulated analyte. In some embodiments, transformation of the data can be applied prior to or after taking the ratio, as disclosed herein.

As used herein, the term “reversal pair” refers to biomarkers in pairs that exhibit a change in value between the classes being compared. The detection of reversals in analyte (i.e. miRNA) concentrations eliminates the need for data normalization or the establishment of population-wide thresholds. Encompassed within the definition of any reversal pair is the corresponding reversal pair wherein individual biomarkers are switched between the numerator and denominator. One skilled in the art will appreciate that such a corresponding reversal pair is equally informative with regard to its predictive power.

The term “reversal value” refers to the ratio of the abundance of two analytes and serves to both normalize variability and amplify diagnostic signal. In some embodiments, a reversal value refers to the ratio of the abundance of an up-regulated (interchangeably referred to as “over-abundant,” up-regulation as used herein simply refers to an observation of abundance) analyte over a down-regulated analyte (interchangeably referred to as “under-abundant,” down-regulation as used herein simply refers to an observation of relative abundance). In some embodiments, a reversal value refers to the ratio of an up-regulated analyte over an up-regulated analyte, where one analyte differs in the degree of up-regulation relative the other analyte. In some embodiments, a reversal value refers to the ratio a down-regulated analyte over a down-regulated analyte, where one analyte differs in the degree of down-regulation relative the other analyte. In some embodiments a reversal value refers to the ratio of a regulated analyte (up or down) and an analyte that is un-regulated. In this case the un-regulated analyte can still serve to normalize. In some embodiments, a reversal value refers to the ratio of two analytes that are un-regulated or whose directions of regulation are unknown. In this case, the un-regulated analytes can still serve to normalize each other and to reveal a diagnostic signal.

One advantageous aspect of a reversal is the presence of complementary information in the two analytes, so that the combination of the two is more diagnostic of the condition of interest than either one alone. Preferably the combination of the two analytes increases signal-to-noise ratio by compensating for biomedical conditions not of interest, pre-analytic variability and/or analytic variability. Out of all the possible reversals within a narrow window, a subset can be selected based on individual univariate performance. Additionally, a subset can be selected based on bivariate or multivariate performance in a training set, with testing on held-out data or on bootstrap iterations. For example, logistic or linear regression models can be trained, optionally with parameter shrinkage by L1 or L2 or other penalties, and tested in leave-one-out, leave-pair-out or leave-fold-out cross-validation, or in bootstrap sampling with replacement, or in a held-out data set. As disclosed herein, the ratio of the abundance of two analytes, for example, the ratio of an up-regulated biomarker over a down-regulated biomarker, referred herein as a reversal value, can be used to identify robust and accurate classifiers and predict a pregnant female's risk of developing placental dysfunction later in the pregnancy

Use of a ratio of biomarkers in the methods disclosed herein corrects for variability that is the result of human manipulation after the removal of the biological sample from the pregnant female. Such variability can be introduced, for example, during sample collection, processing, depletion, digestion or any other step of the methods used to measure the biomarkers present in a sample and is independent of how the biomarkers behave in nature. Accordingly, the invention generally encompasses the use of a reversal pair in a method of diagnosis or prognosis to reduce variability and/or amplify, normalize or clarify diagnostic signal.

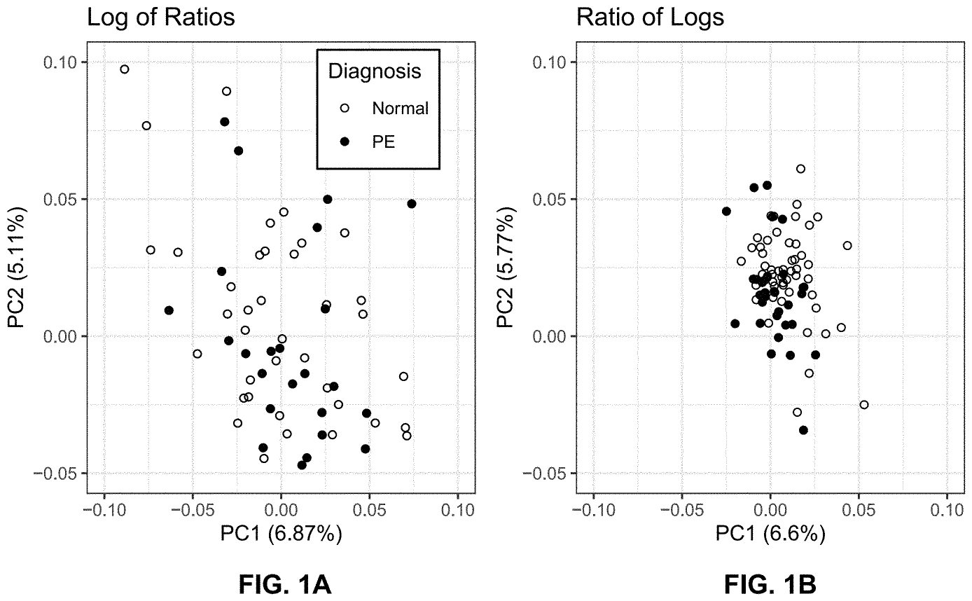

While the term reversal value can refer to the ratio of the abundance of an up regulated analyte over a down regulated analyte and serves to both normalize variability and amplify diagnostic signal, it is also contemplated that a pair of biomarkers of the invention could be treated in a classifier by any other means, for example, by subtraction, addition or multiplication of abundances. In addition, it is contemplated that a value can be mathematically converted to a different value and used to determine a ratio. For example, as disclosed herein, reversals can be constructed as the ratios of the logarithm (log) values. Similarly, ratios can be mathematically converted, for example, as the log of the ratioed values (see Example 2 and FIG. 1). The methods disclosed herein encompass the measurement of biomarker pairs by such other means. A person skilled in the art will readily understand suitable data transformations that can be applied to identify biomarkers predictive of placental dysfunction, including the data transformations disclosed herein. Exemplary transformations include, but are not limited to, box-cox, root, inverse, rank and log. Such data transformations are well known in the art, for example, root (where the root transformation is selected as appropriate for the data set, such as 2, 3, 4, and higher, as appropriate), inverse (1/X), rank (assigning to an ordered list based on appropriate criteria), and so forth, as is well known in the art.

This method is advantageous because it provides the simplest possible classifier that may be independent of data normalization, helps to avoid overfitting, and results in a very simple experimental test that is easy to implement in the clinic. In some uses of the term “reversal” it refers to the identification of analyte pairs where the relative expression (rank order) of each member of a pair reverses in the two conditions studied (e.g. cancer vs not cancer, placental dysfunction vs not). Reversal, as it is used here, allows for there to be opposing regulation of the two members of the pair (e.g., up or down), but does not require that their rank order in abundance to “reverse” in the different clinical conditions. The use of marker pairs based on changes in reversal values that are independent of data normalization enabled the development of the clinically relevant biomarkers disclosed herein. Because quantification of any single protein is subject to uncertainties caused by measurement variability, normal fluctuations, and individual related variation in baseline expression, identification of pairs of markers that may be under coordinated, systematic regulation enables robust methods for diagnosis and prognosis.

While the specification discloses embodiments directed to measuring the particular pairs of biomarkers disclosed in Tables 7-10 or 16-18, the invention is not restricted to the particular pairs recited in Tables 7-10 or 16-18 and individual biomarkers disclosed herein, for example, in Tables 3-6, 15, 17 or 18, as well as any pair or panel of the individual biomarkers is also encompassed by the present invention, as are methods comprising one or more pairs of biomarkers, for example, pairs of biomarkers comprising the biomarkers of any one of Tables 3-11 or 15-18. It is understood that the univariate and bivariate biomarkers disclosed herein, for example, in any one of Tables 3-11 or 15-18, can be used as biomarkers, either singly, in combinations of 2 or more biomarkers, as panels, or in combination with other variables (for example, proteins, metabolites, other molecules, clinical factors, and/or demographic factors) to predict placental dysfunction, such as preeclampsia, as disclosed herein. A person skilled in the art can readily contemplate these and/or additional parameters that can be combined with the biomarkers disclosed herein to predict placental dysfunction.

In one embodiment, the biological sample is selected from the group consisting of whole blood, plasma, and serum. In one embodiment, the biological sample is serum. In one embodiment, the sample is obtained between 18 and 21 weeks of gestational age. In an additional embodiment, the sample is obtained between 23 and 28 weeks of gestational age. In a further embodiment, the sample is obtained between 18 and 28 weeks of gestational age. In some embodiments, the sample is obtained between 119 and 202 days of gestational age. In further embodiments, the sample is obtained between 119 and 152 days of gestational age. In some embodiments, the sample is obtained between 138 and 172 days of gestational age. In further embodiments, the sample is obtained between 156 and 196 days of gestational age.

In addition to biomarkers, measurable features can further include clinical variables including, for example, maternal characteristics, age, race, ethnicity, medical history, past pregnancy history, obstetrical history. For a risk indicium, a measurable feature can include, for example, previous low birth weight or preterm delivery, multiple 2nd trimester spontaneous abortions, prior first trimester induced abortion, familial and intergenerational factors, history of infertility, nulliparity, placental abnormalities, cervical and uterine anomalies, short cervical length measurements, gestational bleeding, intrauterine growth restriction, in utero diethylstilbestrol exposure, multiple gestations, infant sex, short stature, low prepregnancy weight/low body mass index, diabetes, hypertension, urogenital infections, hypothyroidism, asthma, low educational attainment, cigarette smoking, drug use and alcohol consumption.

In some embodiments, the methods of the invention comprise calculation of body mass index (BMI).

As used herein, the term “risk score” refers to a score that can be assigned based on comparing the amount of one or more biomarkers or reversal values in a biological sample obtained from a pregnant female to a standard or reference score that represents an average amount of the one or more biomarkers calculated from biological samples obtained from a random pool of pregnant females. Alternatively, the calculated “risk score” can be compared to the average population risk (prevalence of the outcomes). As will be apparent to one of skill in the art, a risk score can represent the positive predictive value (PPV) of the pregnant female's one or more biomarkers or reversal values for occurrence of the event, i.e., placental dysfunction. A risk score can also represent the probability of occurrence of the event given the pregnant female's one or more biomarkers or reversal values. In a simple embodiment, the pregnant female's risk prior to measurement of biomarkers (pre-test risk) is assigned to be the average population risk (prevalence of the event). Her risk is updated upon measurement of biomarkers and to a post-test risk by calculation of the risk score. An individual pre-test risk can also be assigned to a pregnant female based her standard clinical and demographic data, or on individual, family or ancestral health history or genetic data. For example, a pregnant female with a history of prior preeclampsia may have a greater individual risk for placental dysfunction than the average population risk. The calculated risk based on biomarkers can then be an updated (post-test) risk for the current pregnancy, beyond that individual pre-test risk. A calculated risk of placental dysfunction can also be updated by events or information gathered after the test is applied in the current pregnancy. For example, a pregnant female with a calculated risk of placental dysfunction of 30%, but exhibiting later signs or symptoms (e.g., moderately elevated blood pressure) may have an even higher risk of placental dysfunction (>30%) given the combination of the test and the later sign or symptom. In some embodiments, the risk score is expressed as the log of the reversal value, i.e. the ratio of the relative intensities of the individual biomarkers. One skilled in the art will appreciate that a risk score can be expressed based on a various data transformations as well as being expressed as the ratio itself. Furthermore, with particular regard to reversal pairs, one skilled in the art will appreciate that any ratio is equally informative if the biomarkers in the numerator and denominator are switched or that related data transformations (e.g., subtraction) are applied. Because the level of a biomarker may not be static throughout pregnancy, a standard or reference score has to have been obtained for the gestational time point that corresponds to that of the pregnant female at the time the sample was taken. The standard or reference score can be predetermined and built into a predictor model such that the comparison is indirect rather than actually performed every time the probability is determined for a subject. A risk score can be a standard (e.g., a number) or a threshold (e.g., a line on a graph). The value of the risk score correlates to the deviation, upwards or downwards, from the average amount of the one or more biomarkers calculated from biological samples obtained from a random pool of pregnant females.

In some embodiments, the methods of the invention can be practiced with samples obtained from pregnant females with a specified BMI. Briefly, BMI is an individual's weight in kilograms divided by the square of height in meters. BMI does not measure body fat directly, but research has shown that BMI is correlated with more direct measures of body fat obtained from skinfold thickness measurements, bioelectrical impedance, densitometry (underwater weighing), dual energy x-ray absorptiometry (DXA) and other methods. Furthermore, BMI appears to be as strongly correlated with various metabolic and disease outcome as are these more direct measures of body fatness. Generally, an individual with a BMI below 18.5 is considered underweight, an individual with a BMI of equal or greater than 18.5 to 24.9 normal weight, while an individual with a BMI of equal or greater than 25.0 to 29.9 is considered overweight and an individual with a BMI of equal or greater than 30.0 is considered obese. In some embodiments, the predictive performance of the claimed methods can be improved with a BMI stratification of equal or greater than 18, equal or greater than 19, equal or greater than 20, equal or greater than 21, equal or greater than 22, equal or greater than 23, equal or greater than 24, equal or greater than 25, equal or greater than 26, equal or greater than 27, equal or greater than 28, equal or greater than 29 or equal or greater than 30. In other embodiments, the predictive performance of the claimed methods can be improved with a BMI stratification of equal or less than 18, equal or less than 19, equal or less than 20, equal or less than 21, equal or less than 22, equal or less than 23, equal or less than 24, equal or less than 25, equal or less than 26, equal or less than 27, equal or less than 28, equal or less than 29 or equal or less than 30.

In the context of the present invention, the term “biological sample,” encompasses any sample that is taken from pregnant female and contains one or more of the biomarkers disclosed herein. Suitable samples in the context of the present invention include, for example, blood, plasma, serum, amniotic fluid, vaginal secretions, saliva, and urine. In some embodiments, the biological sample is selected from the group consisting of whole blood, plasma, and serum. In a particular embodiment, the biological sample is serum. As will be appreciated by those skilled in the art, a biological sample can include any fraction or component of blood, without limitation, T cells, monocytes, neutrophils, erythrocytes, platelets and microvesicles such as exosomes and exosome-like vesicles. In a particular embodiment, the biological sample is serum.

The term “amount” or “level” as used herein refers to a quantity of a biomarker that is detectable or measurable in a biological sample and/or control. The quantity of a biomarker can be, for example, the quantity of nucleic acid (i.e. miRNA), the quantity of a polypeptide, the quantity of nucleic acid, or the quantity of a fragment or surrogate. The term can alternatively include combinations thereof. The term “amount” or “level” of a biomarker is a measurable feature of that biomarker.

In some embodiments, nucleic acid amplification methods can be used to detect a polynucleotide biomarker. For example, the oligonucleotide primers and probes of the present invention can be used in amplification and detection methods that use nucleic acid substrates isolated by any of a variety of well-known and established methodologies (e.g., Sambrook et al., Molecular Cloning, A laboratory Manual, pp. 7.37-7.57 (2nd ed., 1989); Lin et al., in Diagnostic Molecular Microbiology, Principles and Applications, pp. 605-16 (Persing et al., eds. (1993); Ausubel et al., Current Protocols in Molecular Biology (2001 and subsequent updates)). Methods for amplifying nucleic acids include, but are not limited to, for example the polymerase chain reaction (PCR) and reverse transcription PCR (RT-PCR) (see e.g., U.S. Pat. Nos. 4,683,195; 4,683,202; 4,800,159; 4,965,188), ligase chain reaction (LCR) (see, e.g., Weiss, Science 254:1292-93 (1991)), strand displacement amplification (SDA) (see e.g., Walker et al., Proc. Natl. Acad. Sci. USA 89:392-396 (1992); U.S. Pat. Nos. 5,270,184 and 5,455,166), Thermophilic SDA (tSDA) (see e.g., European Pat. No. 0 684 315), digital PCR (see, e.g., Salipante et al., Clin. Chem. doi: 10.1373/clinchem.2019.304048 (2019)), and methods described in U.S. Pat. No. 5,130,238; Lizardi et al., BioTechnol. 6:1197-1202 (1988); Kwoh et al., Proc. Natl. Acad. Sci. USA 86:1173-77 (1989); Guatelli et al., Proc. Natl. Acad. Sci. USA 87:1874-78 (1990); U.S. Pat. Nos. 5,480,784; 5,399,491; US Publication No. 2006/46265.

In some embodiments, measuring mRNA in a biological sample can be used as a surrogate for detection of the level of the corresponding protein biomarker in a biological sample. Thus, any of the biomarkers, biomarker pairs or biomarker reversal panels described herein can also be detected by detecting the appropriate RNA. Levels of mRNA can measured by reverse transcription quantitative polymerase chain reaction (RT-PCR followed with qPCR). RT-PCR is used to create a cDNA from the mRNA. The cDNA can be used in a qPCR assay to produce fluorescence as the DNA amplification process progresses. By comparison to a standard curve, qPCR can produce an absolute measurement such as number of copies of mRNA per cell. Digital PCR is a special case of qPCR, where PCR is performed in many discrete partitions of the sample, and can be more sensitive and reliable than traditional qPCR (see, e.g., Salipante et al., Clin. Chem. doi: 10.1373/clinchem.2019.304048 (2019)). Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

In one aspect, the invention provides a panel of isolated nucleic acid biomarkers comprising two or more of the nucleic acid biomarkers set forth in Tables 3-11 or 15-18.

In a further aspect, the invention provides a composition of labeled and/or amplified nucleic acid molecules, for example, amplified labeled nucleic acid molecules, that correspond to two or more of the nucleic acid biomarkers set forth in Tables 3-11 or 15-18.

In a further aspect, the invention provides a panel of isolated nucleic acid biomarkers comprising two or more of the nucleic acid biomarkers set forth in Tables 3-6, 15, 17 or 18.

In an additional aspect, the invention provides a composition of labeled and/or amplified nucleic acid molecules, for example, amplified labeled nucleic acid molecules, that correspond to two or more of the nucleic acid biomarkers set forth in Tables 3-6, 15, 17 or 18.

In additional embodiments, the invention provides a biomarker panel comprising two or more of the isolated nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941. In some embodiments, the biomarker panel comprises isolated nucleic acid biomarkers comprising hsa-miR-331-3p and/or hsa-miR-941. In some embodiments, the biomarker panel comprises isolated nucleic acid biomarkers comprising hsa-miR-423-3p, hsa-miR-516b-5p, hsa-miR-4732-5p, and/or hsa-miR-1273h-3p. In some embodiments, the biomarker panel comprises isolated nucleic acid biomarkers comprising hsa-miR-4732-5p, hsa-miR-516b-5p, and/or hsa-miR-941. In additional embodiments, the biomarker panel comprises isolated nucleic acid biomarkers comprising hsa-miR-155-5p, hsa-miR-331-3p, hsa-miR-1273h-3p, and/or hsa-miR-516b-5p. In some embodiments, the biomarker panel comprises isolated nucleic acid biomarkers comprising hsa-miR-516b-5p, and/or hsa-miR-941.

In a further aspect, the invention provides a composition of labeled and/or amplified nucleic acid molecules, for example, amplified labeled nucleic acid molecules, that correspond to two or more of the nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941. In an additional aspect, the invention provides a composition of labeled and/or amplified nucleic acid molecules, for example, amplified labeled nucleic acid molecules, that correspond to nucleic acid biomarkers hsa-miR-331-3p and/or hsa-miR-941. In an additional aspect, the invention provides a composition of labeled and/or amplified nucleic acid molecules, for example, amplified labeled nucleic acid molecules, that correspond to nucleic acid biomarkers hsa-miR-423-3p, hsa-miR-516b-5p, hsa-miR-4732-5p, and/or hsa-miR-1273h-3p. In an additional aspect, the invention provides a composition of labeled and/or amplified nucleic acid molecules, for example, amplified labeled nucleic acid molecules, that correspond to nucleic acid biomarkers hsa-miR-4732-5p, hsa-miR-516b-5p, and/or hsa-miR-941. In an additional aspect, the invention provides a composition of labeled and/or amplified nucleic acid molecules, for example, amplified labeled nucleic acid molecules, that correspond to nucleic acid biomarkers hsa-miR-155-5p, hsa-miR-331-3p, hsa-miR-1273h-3p, and/or hsa-miR-516b-5p. In some embodiments, the composition comprises isolated nucleic acid biomarkers comprising hsa-miR-516b-5p, and/or hsa-miR-941.

In one aspect, the invention provides a pair of biomarker selected from the group consisting of the nucleic acid biomarkers set forth in Tables 3-11 or 15-18.

In a further aspect, the invention provides a composition of labeled and/or amplified nucleic acid molecules, for example, amplified labeled nucleic acid molecules, that correspond to a pair of biomarker selected from the group consisting of the nucleic acid biomarkers set forth in Tables 3-11 or 15-18.

In further embodiments, the invention provides a pair of nucleic acid biomarkers selected from the group consisting of the biomarker pairs set forth in Tables 7-10 or 16-18.

In a further aspect, the invention provides a composition of labeled and/or amplified nucleic acid molecules, for example, amplified labeled nucleic acid molecules, that correspond to a pair of biomarker selected from the group consisting of the biomarker pairs set forth in Tables 7-10 or 16-18.

In further embodiments, the invention provides a pair of nucleic acid biomarkers, or a panel of isolated nucleic acid biomarkers comprising a pair of biomarkers, where the pair of biomarkers is selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p. In some embodiments, the pair of biomarkers is selected from the group consisting of hsa-miR-127-3p/hsa-miR-485-5p, hsa-miR-4732-3p/hsa-miR-941, hsa-miR-1273h-3p/hsa-miR-3173-5p, hsa-miR-155-5p/hsa-miR-3173-5p, hsa-miR-150-3p/hsa-miR-193b-5p, hsa-miR-1285-3p/hsa-mir-378c, hsa-miR-4732-3p/hsa-miR-381-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-25-3p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-let-7i-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-451a/hsa-miR-155-5p, hsa-miR-125a-5p/hsa-miR-155-5p, hsa-miR-99a-5p/hsa-miR-155-5p, hsa-let-7b-5p/hsa-miR-155-5p, hsa-miR-516b-5p/hsa-miR-155-5p, and hsa-miR-363-3p/hsa-miR-155-5p.

In a further aspect, the invention provides a composition of labeled and/or amplified nucleic acid molecules, for example, amplified labeled nucleic acid molecules, that correspond to a pair of biomarker selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p. In some embodiments, the pair of biomarkers is selected from the group consisting of hsa-miR-127-3p/hsa-miR-485-5p, hsa-miR-4732-3p/hsa-miR-941, hsa-miR-1273h-3p/hsa-miR-3173-5p, hsa-miR-155-5p/hsa-miR-3173-5p, hsa-miR-150-3p/hsa-miR-193b-5p, hsa-miR-1285-3p/hsa-mir-378c, hsa-miR-4732-3p/hsa-miR-381-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-25-3p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-let-7i-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-451a/hsa-miR-155-5p, hsa-miR-125a-5p/hsa-miR-155-5p, hsa-miR-99a-5p/hsa-miR-155-5p, hsa-let-7b-5p/hsa-miR-155-5p, hsa-miR-516b-5p/hsa-miR-155-5p, and hsa-miR-363-3p/hsa-miR-155-5p.

In some embodiments, a pair of nucleic acid biomarkers is selected from the group consisting of hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p. In a further aspect, the invention provides a composition of labeled and/or amplified nucleic acid molecules, for example, amplified labeled nucleic acid molecules, that correspond to a pair of nucleic acid biomarkers consisting of hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p.

In further embodiments, the invention provides a pair of nucleic acid biomarkers selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p.

In a further aspect, the invention provides a composition of labeled and/or amplified nucleic acid molecules, for example, amplified labeled nucleic acid molecules, that correspond to a pair of biomarker selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p.

In further embodiments, the invention provides a pair of nucleic acid biomarkers selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p.

In a further aspect, the invention provides a composition of labeled and/or amplified nucleic acid molecules, for example, amplified labeled nucleic acid molecules, that correspond to a pair of biomarker selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p.

In additional embodiments, the invention provides a pair of nucleic acid biomarkers selected from the group consisting of hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

In a further aspect, the invention provides a composition of labeled and/or amplified nucleic acid molecules, for example, amplified labeled nucleic acid molecules, that correspond to a pair of biomarker selected from the group consisting of hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

Also provided by the invention is a method of determining a pregnant female's risk of developing placental dysfunction later in the pregnancy comprising measuring the amount of two or more of the nucleic acid biomarkers listed in Tables 3-11 or 15-18 in a biological sample obtained from the pregnant female, and calculating a risk score based upon the measured amounts of the nucleic acid biomarkers to determine the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to two or more of the nucleic acid biomarkers set forth in Tables 3-11 or 15-18 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction.

Also provided by the invention is a method of determining a pregnant female's risk of developing placental dysfunction later in the pregnancy comprising measuring the amount of two or more of the nucleic acid biomarkers listed in Tables 3-6, 15, 17 or 18 in a biological sample obtained from the pregnant female, and calculating a risk score based upon the measured amounts of the nucleic acid biomarkers to determine the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to two or more of the nucleic acid biomarkers set forth in Tables 3-6, 15, 17 or 18 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction.

Also provided by the invention is a method of determining a pregnant female's risk of developing placental dysfunction later in the pregnancy comprising measuring the amount of two or more of the nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941 in a biological sample obtained from the pregnant female, and calculating a risk score based upon the measured amounts of the nucleic acid biomarkers to determine the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to two or more of the nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction.

Also provided by the invention is a method of determining a pregnant female's risk of developing placental dysfunction later in the pregnancy comprising measuring the amount of hsa-miR-331-3p and/or hsa-miR-941 in a biological sample obtained from the pregnant female, and calculating a risk score based upon the measured amounts of the nucleic acid biomarkers to determine the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-331-3p and/or hsa-miR-941 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction.

Also provided by the invention is a method of determining a pregnant female's risk of developing placental dysfunction later in the pregnancy comprising measuring the amount of hsa-miR-423-3p, hsa-miR-516b-5p, hsa-miR-4732-5p, and/or hsa-miR-1273h-3p in a biological sample obtained from the pregnant female, and calculating a risk score based upon the measured amounts of the nucleic acid biomarkers to determine the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-423-3p, hsa-miR-516b-5p, hsa-miR-4732-5p, and/or hsa-miR-1273h-3p in a biological sample obtained from the pregnant female; measuring the levels of expression of the labeled and/or amplified nucleic acid molecules, for example, amplified labeled nucleic acid molecules,; calculating a risk score based upon the measured levels of the labeled and/or amplified nucleic acid molecules, for example, amplified labeled nucleic acid molecules, to determine the pregnant female's risk of developing placental dysfunction.

Also provided by the invention is a method of determining a pregnant female's risk of developing placental dysfunction later in the pregnancy comprising measuring the amount of hsa-miR-4732-5p, hsa-miR-516b-5p, and/or hsa-miR-941 in a biological sample obtained from the pregnant female, and calculating a risk score based upon the measured amounts of the nucleic acid biomarkers to determine the pregnant female's risk of developing placental dysfunction. In some embodiments, hsa-miR-516b-5p, and/or hsa-miR-941 is measured.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-4732-5p, hsa-miR-516b-5p, and/or hsa-miR-941 in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the labeled and/or amplified nucleic acid molecules, for example, amplified labeled nucleic acid molecules, correspond to hsa-miR-516b-5p, and/or hsa-miR-941 is measured.

Also provided by the invention is a method of determining a pregnant female's risk of developing placental dysfunction later in the pregnancy comprising measuring the amount of hsa-miR-155-5p, hsa-miR-331-3p, hsa-miR-1273h-3p, and/or hsa-miR-516b-5p in a biological sample obtained from the pregnant female, and calculating a risk score based upon the measured amounts of the nucleic acid biomarkers to determine the pregnant female's risk of developing placental dysfunction.

In some embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to hsa-miR-155-5p, hsa-miR-331-3p, hsa-miR-1273h-3p, and/or hsa-miR-516b-5p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction.

In some embodiments, the invention provides a method of determining a pregnant female's risk of developing placental dysfunction later in the pregnancy comprising measuring the amount of a pair of nucleic acid biomarkers selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p in a biological sample obtained from the pregnant female, and calculating a risk score based upon the measured amounts of the nucleic acid biomarkers to determine the pregnant female's risk of developing placental dysfunction. In some embodiments, the pair of biomarkers is selected from the group consisting of hsa-miR-127-3p/hsa-miR-485-5p, hsa-miR-4732-3p/hsa-miR-941, hsa-miR-1273h-3p/hsa-miR-3173-5p, hsa-miR-155-5p/hsa-miR-3173-5p, hsa-miR-150-3p/hsa-miR-193b-5p, hsa-miR-1285-3p/hsa-mir-378c, hsa-miR-4732-3p/hsa-miR-381-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-25-3p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-let-7i-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-451a/hsa-miR-155-5p, hsa-miR-125a-5p/hsa-miR-155-5p, hsa-miR-99a-5p/hsa-miR-155-5p, hsa-let-7b-5p/hsa-miR-155-5p, hsa-miR-516b-5p/hsa-miR-155-5p, and hsa-miR-363-3p/hsa-miR-155-5p.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction. In some embodiments, the pair of biomarkers is selected from the group consisting of hsa-miR-127-3p/hsa-miR-485-5p, hsa-miR-4732-3p/hsa-miR-941, hsa-miR-1273h-3p/hsa-miR-3173-5p, hsa-miR-155-5p/hsa-miR-3173-5p, hsa-miR-150-3p/hsa-miR-193b-5p, hsa-miR-1285-3p/hsa-mir-378c, hsa-miR-4732-3p/hsa-miR-381-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-25-3p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-let-7i-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-451a/hsa-miR-155-5p, hsa-miR-125a-5p/hsa-miR-155-5p, hsa-miR-99a-5p/hsa-miR-155-5p, hsa-let-7b-5p/hsa-miR-155-5p, hsa-miR-516b-5p/hsa-miR-155-5p, and hsa-miR-363-3p/hsa-miR-155-5p.

In some embodiments, the invention provides a method of determining a pregnant female's risk of developing placental dysfunction later in the pregnancy comprising measuring the amount of a pair of nucleic acid biomarkers hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p in a biological sample obtained from the pregnant female, and calculating a risk score based upon the measured amounts of the nucleic acid biomarkers to determine the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction.

In further embodiments, the invention provides a method of determining a pregnant female's risk of developing placental dysfunction later in the pregnancy comprising measuring the amount of a pair of nucleic acid biomarkers selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p in a biological sample obtained from the pregnant female, and calculating a risk score based upon the measured amounts of the nucleic acid biomarkers to determine the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p in a biological sample obtained from the pregnant female; measuring the levels of expression of the labeled and/or amplified nucleic acid molecules, for example, amplified labeled nucleic acid molecules,; calculating a risk score based upon the measured levels of the labeled and/or amplified nucleic acid molecules, for example, amplified labeled nucleic acid molecules, to determine the pregnant female's risk of developing placental dysfunction.

In further embodiments, the invention provides a method of determining a pregnant female's risk of developing placental dysfunction later in the pregnancy comprising measuring the amount of a pair of nucleic acid biomarkers selected from the group consisting of hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c in a biological sample obtained from the pregnant female, and calculating a risk score based upon the measured amounts of the nucleic acid biomarkers to determine the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction.

In further embodiments, the invention provides a method of determining a pregnant female's risk of developing placental dysfunction later in the pregnancy comprising measuring the amount of a pair of nucleic acid biomarkers selected from the group consisting of hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p in a biological sample obtained from the pregnant female, and calculating a risk score based upon the measured amounts of the nucleic acid biomarkers to determine the pregnant female's risk of developing placental dysfunction.

In additional embodiments, the invention provides a method of determining a pregnant female's risk of developing placental dysfunction later in the pregnancy comprising producing labeled and/or amplified nucleic acid molecules, for example, amplified labeled nucleic acid molecules, that correspond to a pair of nucleic acid biomarkers selected from the group consisting of hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p in a biological sample obtained from the pregnant female; measuring the levels of expression of the labeled and/or amplified nucleic acid molecules, for example, amplified labeled nucleic acid molecules; calculating a risk score based upon the measured levels of the labeled and/or amplified nucleic acid molecules, for example, amplified labeled nucleic acid molecules, to determine the pregnant female's risk of developing placental dysfunction.

The quantitation of biomarkers in a biological sample can be determined, without limitation, by the methods described above as well as any other method known in the art. The quantitative data thus obtained is then subjected to an analytic classification process. In such a process, the raw data is manipulated according to an algorithm, where the algorithm has been pre-defined by a training set of data, for example as described in the examples provided herein. An algorithm can utilize the training set of data provided herein, or can utilize the guidelines provided herein to generate an algorithm with a different set of data.

An analytic classification process can use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc.

Classification can be made according to predictive modeling methods that set a threshold for determining the probability that a sample belongs to a given class. The probability preferably is at least 50%, or at least 60%, or at least 70%, or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUROC (area under the ROC curve) or accuracy, of a particular value, or range of values. Area under the curve measures are useful for comparing the accuracy of a classifier across the complete data range. Classifiers with a greater AUC have a greater capacity to classify unknowns correctly between two groups of interest. In some embodiments, a desired quality threshold is a predictive model that will classify a sample with an accuracy of at least about 0.5, at least about 0.55, at least about 0.6, at least about 0.7, at least about 0.75, at least about 0.8, at least about 0.85, at least about 0.9, at least about 0.95, or higher. As an alternative measure, a desired quality threshold can refer to a predictive model that will classify a sample with an AUC of at least about 0.7, at least about 0.75, at least about 0.8, at least about 0.85, at least about 0.9, or higher.

As is known in the art, the relative sensitivity and specificity of a predictive model can be adjusted to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about 0.7, at least about 0.75, at least about 0.8, at least about 0.85, at least about 0.9, or higher.

The raw data can be initially analyzed by measuring the values for each biomarker, usually in triplicate or in multiple triplicates. The data can be manipulated, for example, raw data can be transformed using standard curves, and the average of triplicate measurements used to calculate the average and standard deviation for each patient. These values can be transformed before being used in the models, e.g. log-transformed, Box-Cox transformed (Box and Cox, Royal Stat. Soc., Series B, 26:211-246(1964). The data are then input into a predictive model, which will classify the sample according to the state. The resulting information can be communicated to a patient or health care provider.

To generate a predictive model for determining the risk of developing placental dysfunction later in the pregnancy a robust data set, comprising known control samples and samples corresponding to the birth classification of interest is used in a training set. A sample size can be selected using generally accepted criteria. As discussed above, different statistical methods can be used to obtain a highly accurate predictive model.

In one embodiment, hierarchical clustering is performed in the derivation of a predictive model, where the Pearson correlation is employed as the clustering metric. One approach is to consider a given birth dataset as a “learning sample” in a problem of “supervised learning.” CART is a standard in applications to medicine (Singer, Recursive Partitioning in the Health Sciences, Springer(1999)) and can be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

This approach led to what is termed FlexTree (Huang, Proc. Nat. Acad. Sci. U.S.A 101:10529-10534(2004)). FlexTree performs very well in simulations and when applied to multiple forms of data and is useful for practicing the claimed methods. Software automating FlexTree has been developed. Alternatively, LARTree or LART can be used (Turnbull (2005) Classification Trees with Subset Analysis Selection by the Lasso, Stanford University). The name reflects binary trees, as in CART and FlexTree; the lasso, as has been noted; and the implementation of the lasso through what is termed LARS by Efron et al. (2004) Annals of Statistics 32:407-451 (2004). See, also, Huang et al., Proc. Natl. Acad. Sci. USA. 101(29):10529-34 (2004). Other methods of analysis that can be used include logic regression. One method of logic regression Ruczinski, Journal of Computational and Graphical Statistics 12:475-512 (2003). Logic regression resembles CART in that its classifier can be displayed as a binary tree. It is different in that each node has Boolean statements about features that are more general than the simple “and” statements produced by CART.

Another approach is that of nearest shrunken centroids (Tibshirani, Proc. Natl. Acad. Sci. U.S.A 99:6567-72(2002)). The technology is k-means-like, but has the advantage that by shrinking cluster centers, one automatically selects features, as is the case in the lasso, to focus attention on small numbers of those that are informative. The approach is available as PAM software and is widely used. Two further sets of algorithms that can be used are random forests (Breiman, Machine Learning 45:5-32 (2001)) and MART (Hastie, The Elements of Statistical Learning, Springer (2001)). These two methods are known in the art as “committee methods,” that involve predictors that “vote” on outcome.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (Tusher et al., Proc. Natl. Acad. Sci. U.S.A 98, 5116-21 (2001)). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles. Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pair wise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

In an alternative analytical approach, variables chosen in the cross-sectional analysis are separately employed as predictors in a time-to-event analysis (survival analysis), where the event is the occurrence of preterm birth, and subjects with no event are considered censored at the time of giving birth. Given the specific pregnancy outcome (preterm birth event or no event), the random lengths of time each patient will be observed, and selection of proteomic and other features, a parametric approach to analyzing survival can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and corresponding functions are available with this model.

In addition the Cox models can be used, especially since reductions of numbers of covariates to manageable size with the lasso will significantly simplify the analysis, allowing the possibility of a nonparametric or semi-parametric approach to prediction of time to preterm birth. These statistical tools are known in the art and applicable to all manner of proteomic data. A set of biomarker, clinical and genetic data that can be easily determined, and that is highly informative regarding the probability for preterm birth and predicted time to a preterm birth event in said pregnant female is provided. Also, algorithms provide information regarding the probability for preterm birth in the pregnant female.

Survival analyses are commonly used to understand time to occurrence of an event of interest such as birth or death. Commonly, the Kaplan-Meier estimator is used to estimate the survival function, while Cox proportional hazards models are used to estimate the effects of covariates on the hazard of event occurrence. These models conventionally assume that survival time is based on risk of exactly one type of event. However a competing risk for a different event may be present that either hinders the observation of an event of interest or modifies the chance that this event occurs. Conventional methods may be inappropriate in the presence of competing risks. Alternative methods appropriate for analysis of competing risks either asses, competing hazards in subdistribution hazards models or cause-specific modified Cox proportional hazards models; or estimate cumulative incidence over competing events (Jason P. Fine & Robert J. Gray. Journal of the American Statistical Association Vol. 94 , Issue 446,1999. A Proportional Hazards Model for the Subdistribution of a Competing Risk).

In the development of a predictive model, it can be desirable to select a subset of markers, i.e. at least 3, at least 4, at least 5, at least 6, up to the complete set of markers. Usually a subset of markers will be chosen that provides for the needs of the quantitative sample analysis, e.g. availability of reagents, convenience of quantitation, etc., while maintaining a highly accurate predictive model. The selection of a number of informative markers for building classification models requires the definition of a performance metric and a user-defined threshold for producing a model with useful predictive ability based on this metric. For example, the performance metric can be the AUC, the sensitivity and/or specificity of the prediction as well as the overall accuracy of the prediction model.

As will be understood by those skilled in the art, an analytic classification process can use any one of a variety of statistical analytic methods to manipulate the quantitative data and provide for classification of the sample. Examples of useful methods include, without limitation, linear discriminant analysis, recursive feature elimination, a prediction analysis of microarray, a logistic regression, a CART algorithm, a FlexTree algorithm, a LART algorithm, a random forest algorithm, a MART algorithm, and machine learning algorithms. Various methods are used in a training model. The selection of a subset of markers can be for a forward selection or a backward selection of a marker subset. The number of markers can be selected that will optimize the performance of a model without the use of all the markers. One way to define the optimum number of terms is to choose the number of terms that produce a model with desired predictive ability (e.g. an AUC>0.75, or equivalent measures of sensitivity/specificity) that lies no more than one standard error from the maximum value obtained for this metric using any combination and number of terms used for the given algorithm.

The biomarkers of the invention, which have been identified in the invention disclosed herein as being useful for predicting placental dysfunction, are known in the art and are readily available in public databases. For example, the human microRNAs disclosed herein as biomarkers useful for determining a pregnant female's risk of developing placental dysfunction are available in mirBase (mirbase.org). The naming convention for microRNAs generally uses “-” in the name of the microRNA, for example, hsa-miR-423-3p. It is noted that in some instances herein a shortened nomenclature is used, in which the “-” is replaced with “.” such as “hsa.miR.423.3p” instead of “hsa-miR-423-3p.” A person skilled in the art will readily understand the nomenclature commonly used for microRNAs and will appreciate that the microRNAs disclosed herein are readily available in public databases (see also Tables 15 and 16, in which mirBase accession numbers have been included). Biomarker pairs are generally denoted herein as a pair separated by a “/”, for example, hsa-miR-127-3p/hsa-miR-485-5p.

In yet another aspect, the invention provides kits for determining a pregnant female's risk of developing placental dysfunction later in the pregnancy. The kit can include one or more agents for detection of biomarkers, a container for holding a biological sample isolated from a pregnant female; and printed instructions for reacting agents with the biological sample or a portion of the biological sample to detect the presence or amount of the isolated biomarkers in the biological sample. The agents can be packaged in separate containers. The kit can further comprise one or more control reference samples and reagents for performing an immunoassay.

The kit can comprise one or more containers for compositions or reagents contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods for determining a pregnant female's risk of developing placental dysfunction later in the pregnancy.

### EXAMPLES

### Example 1. Identification of Extracellular Micro RNA Biomarkers for Identification of Pregnancies at Risk for Placental Dysfunction

This example shows identification of extracellular miRNA biomarkers for prediction of preeclampsia: placental dysfunction affecting maternal blood pressure and renal, liver and central nervous system function.

This is a biomarker discovery study including training and verification phases.

Study Design

Unblinded samples were reserved for the training set. Blinded samples were split between training and verification sets. Additional blinded samples are reserved for future re-verification or validation.

Maternal serum samples were collected from high-risk and average-risk pregnant women between 17-28 weeks, for which pregnancy outcomes are known. The samples were divided into a training set of 141 subjects (49 Preeclampsia/92 Normal) and a verification set of 71 subjects (24 Preeclampsia/47 Normal). GABD for the training and test groups was at a minimum of 120 days to a maximum of 201 days with a mean of 163.6 days.

TABLE 1
Pre-specified subject classifications

| | Range of | Range of | Criterion | Criterion | Criterion | Criterion | Criterion |
|---|---|---|---|---|---|---|---|
| Group | GA at draw | GA at birth | 1 | 2 | 3 | 4 | 5 |
| UCSD | 19-27 | 25-41 | New-onset | Chronic | Chronic | New-onset | New-onset or |
| cases | weeks | weeks | hypertension | hypertension | proteinuria | or chronic | chronic |
| | | | and new- | and new- | and new- | hypertension | proteinuria |
| | | | onset | onset | onset | and new- | and new- |
| | | | proteinuria | proteinuria | hypertension | onset severe | onset severe |
| | | | OR | OR | OR | feature | feature |
| | | | | | | (elevated | (elevated |
| | | | | | | LFTs, | LFTs, |
| | | | | | | elevated Cr, | elevated Cr, |
| | | | | | | low platelets, | low platelets, |
| | | | | | | IUGR) OR | IUGR) |
| Sera | 17 1/7- | 25 5/7- | New-onset | Superimposed | | Proteinuria | |
| PAPR | 28 5/7 | 40 5/7 | hypertension | preeclampsia | | and new- | |
| cases | weeks | | with or | Preeclampsia | | onset severe | |
| | | | without | with or | | feature | |
| | | | severe | without | | (elevated | |
| | | | features OR | severe | | LFTs, | |
| | | | | features, OR | | elevated Cr, | |
| | | | | | | low platelets, | |
| | | | | | | IUGR) | |
| UCSD | 19-27 | 25-41 | No | | | | |
| controls | weeks | weeks | hypertensive | | | | |
| | | | disease | | | | |
| Sera | 17 1/7- | 37 0/7- | | | | | |
| PAPR | 28 5/7 | 41 4/7 | | | | | |
| controls | weeks | weeks | | | | | |

TABLE 2
Sample, maternal, and pregnancy conditions and identifiers

| Field | Description |
|---|---|
| Batch | Small RNAseq analysis batch |
| SampleName | Sample Name |
| GASampleCollection | Gestational Age at Sample Collection |
| GADelivery | Gestational Age at Delivery |
| BMI | First recorded maternal BMI during pregnancy episode |
| MaternalAge | Maternal Age at Delivery |
| Race | Maternal Race (self-reported) |
| Ethnicity | Maternal Ethnicity (self-reported) |
| Birthweight | Birthweight |
| Gender | Fetal sex |
| BW % | Birthweight Percentile (Calculated using Hadlock and Fenton) |
| Diagnosis | Detailed Placental Dysfunction Diagnosis |
| Diagnosis_simple | Simple Placental Dysfunction Diagnosis (Normal, Pree) |
| Diagnosis_mild_severe | Intermediate Placental Dysfunction Diagnosis (Normal, Pree_Mild, |
| | Pree_Severe) |
| Diagnosis_Diabetes | Diabetes Diagnosis (No Diabetes, GDM, T2DM, T1DM) |
| IUGR | Birthweight < 10^{th} percentile (YES, NO) |

Lab Analysis

Total extracellular RNA (exRNA) from each sample was purified and subjected to small RNA sequencing.

Total extracellular RNA was purified from 500 μL serum using the miRNeasy micro kit (Qiagen), followed by the RNA Clean and Concentrator Kit (Zymo), with a final elution volume of 12 μL. 1.2 μL of the resulting exRNA was used to prepare the Small RNAseq libraries using the NEBNext Small RNA Sequencing Library Preparation Kit, using the manufacturer's instruction except for the following: 1. Adapters were diluted 1:6; 2. The reactions were run at 115^{th} volume using a mosquito HTS liquid handler. Up to 384 libraries were prepared in a given batch (some of the libraries for this project were prepared in the same batches as other projects), and libraries were multiplexed using the available 48 NEB Small RNAseq indices. Up to 48 samples were combined per pool, and each pool was size selected using a Pippin Prep (either 177-180 bp, or 125-160 bp). Samples were sequenced on a HiSeq 4000. Each pool of up to 48 samples was loaded onto its own lane, generating at least 350 million single-end 75 bp reads.

Data Analysis

Unblinded UCSD samples were reserved for the training set. Blinded Sera samples were split between training and verification sets, requiring balance between the training and test sets of gestational age at blood draw (GABD), and of the proportions of preeclampsia cases to non-preeclamptic controls across all GABD and in 1- and 3-week windows of GABD. Additional blinded UCSD samples are reserved for future re-verification or validation.

Data preprocessing, including adapter trimming and mapping to miRBase (ref to miRbase version), was performed using the ExceRpt pipeline on the Genboree Workbench (which can be accessed at exRNA.org), to yield Raw Count data.

Further filtering removed individual miRNAs with >70% missing values. Batch normalization was carried out using Variance Stabilizing Transformation and Bias Reduction. The PEER package (Sanger Institute) was run to further to reduce batch effect and amplify preeclampsia signal. Replicate data was then collapsed to single values. AUCs were generated with the pROC package, using the Delong and bootstrap methods to establish the confidence intervals (CIs). Analysis was performed using R 3.4.3 (Robin et al., BMC bioinformatics 12:77 (2011)).

Four windows of blood draw GA were considered: full window (119-202 days), early window (119-152), middle window (138-172 days), and late window (156-196 days).

Univariate miRNA models were fit to the entire dataset range of gestational age at blood draw (GABD) and to early, middle and late GABD windows. Univariate models with significant chi-square p-values between residual and null deviance were selected in each window.

Bivariate reversals (ratios of miRNAs) were selected by ranking performance in bootstrapped resampling with replacement.

Mean and 95% CI of AUC

Pearson correlation of reversal score with diagnosis of Preeclampsia (1) or not (0)

Mean of the differences in reversal score between cases and controls

Numeric performance data were converted to ranks, inverted and summed to produce a final ranking. The top 50 reversals were selected for entire, early, middle and late windows.

As the output of the discovery phase, 60 univariate hypotheses and 200 reversal hypotheses are enumerated in the attached tables.

These hypotheses will be tested en masse for AUC>0.5 (95% CI does not include 0.5) in the Verification phase. Surviving hypotheses will be filtered for overall performance in both the Discovery and Verification sets and for kinetics of performance in these sets.

Top ranking reversals after Verification testing and filtering may be:
a) Formed into tree-like panel hypotheses of robust performance across multiple weeks.
b) Formed into combined protein / RNA hypotheses to the extent that proteomic data are available for Discovery and Verification samples.
c) Re-verified or validated as univariate, bivariate and panel hypotheses in a future small RNA sequencing data set, for example comprising blinded UCSD samples.

TABLE 3
Selected single-miRNA hypotheses in the entire GABD window

| | AUC | | | Significance | | |
|---|---|---|---|---|---|---|
| miRNA | AUC | lo 95% CI | hi 95% CI | Wilcoxon | T-test | Chi-square |
| hsa.miR.30c.5p | 0.662 | 0.565 | 0.759 | 1.6E−03 | 6.0E−03 | 2.8E−04 |
| hsa.miR.1301.3p | 0.658 | 0.560 | 0.757 | 2.0E−03 | 7.7E−03 | 7.1E−04 |
| hsa.miR.23a.3p | 0.660 | 0.568 | 0.753 | 1.8E−03 | 4.2E−03 | 1.4E−03 |
| hsa.miR.6842.3p | 0.643 | 0.550 | 0.736 | 5.3E−03 | 1.0E−02 | 4.7E−03 |
| hsa.miR.485.5p | 0.663 | 0.568 | 0.758 | 1.5E−03 | 8.9E−03 | 1.2E−02 |
| hsa.miR.361.3p | 0.618 | 0.518 | 0.717 | 2.2E−02 | 1.6E−02 | 1.2E−02 |
| hsa.miR.191.5p | 0.618 | 0.518 | 0.719 | 2.1E−02 | 1.7E−02 | 1.2E−02 |
| hsa.miR.4446.3p | 0.653 | 0.556 | 0.749 | 2.9E−03 | 7.6E−03 | 1.2E−02 |
| hsa.miR.6747.3p | 0.637 | 0.538 | 0.736 | 7.6E−03 | 2.1E−02 | 1.5E−02 |
| hsa.miR.409.3p | 0.601 | 0.505 | 0.697 | 4.8E−02 | 6.2E−02 | 2.3E−02 |
| hsa.miR.224.5p | 0.602 | 0.504 | 0.701 | 4.6E−02 | 4.4E−02 | 2.8E−02 |
| hsa.miR.1224.5p | 0.596 | 0.497 | 0.696 | 6.0E−02 | 4.0E−02 | 3.2E−02 |
| hsa.miR.423.3p | 0.601 | 0.501 | 0.700 | 4.9E−02 | 3.7E−02 | 3.9E−02 |
| hsa.miR.941 | 0.645 | 0.552 | 0.738 | 4.6E−03 | 3.5E−02 | 4.2E−02 |

TABLE 4
Selected single-miRNA hypotheses in the early GABD window

| | AUC | | | Significance | | |
|---|---|---|---|---|---|---|
| miRNA | AUC | lo 95% CI | hi 95% CI | Wilcoxon | T-test | Chi-square |
| hsa.miR.30d.5p | 0.774 | 0.618 | 0.931 | 4.5E−03 | 7.0E−03 | 4.8E−03 |
| hsa.miR.1323 | 0.680 | 0.513 | 0.847 | 6.2E−02 | 2.9E−02 | 1.2E−02 |
| hsa.let.7d.3p | 0.712 | 0.540 | 0.884 | 2.8E−02 | 5.7E−02 | 1.5E−02 |
| hsa.miR.191.5p | 0.692 | 0.532 | 0.853 | 4.6E−02 | 9.0E−03 | 1.9E−02 |
| hsa.miR.518e.5p | 0.749 | 0.595 | 0.904 | 9.7E−03 | 3.7E−02 | 2.2E−02 |
| hsa.miR.519a.5p | | | | | | |
| hsa.miR.519b.5p | | | | | | |
| hsa.miR.519c.5p | | | | | | |
| hsa.miR.522.5p | | | | | | |
| hsa.miR.523.5p | | | | | | |
| hsa.miR.516b.5p | 0.809 | 0.664 | 0.954 | 1.4E−03 | 4.2E−02 | 2.4E−02 |
| hsa.miR.26a.5p | 0.705 | 0.540 | 0.870 | 3.4E−02 | 2.7E−02 | 2.5E−02 |
| hsa.miR.99b.5p | 0.655 | 0.454 | 0.856 | 1.1E−01 | 1.1E−01 | 2.8E−02 |
| hsa.miR.18a.3p | 0.727 | 0.555 | 0.899 | 1.9E−02 | 5.2E−02 | 3.0E−02 |
| hsa.miR.1224.5p | 0.710 | 0.536 | 0.884 | 3.0E−02 | 5.3E−02 | 3.5E−02 |
| hsa.miR.142.3p | 0.660 | 0.480 | 0.840 | 9.7E−02 | 6.0E−02 | 3.7E−02 |
| hsa.miR.423.3p | 0.702 | 0.528 | 0.876 | 3.6E−02 | 4.2E−02 | 3.7E−02 |
| hsa.miR.4429 | 0.593 | 0.402 | 0.784 | 3.3E−01 | 1.2E−01 | 4.3E−02 |
| hsa.miR.224.5p | 0.675 | 0.501 | 0.849 | 7.0E−02 | 6.1E−02 | 4.6E−02 |

TABLE 5
Selected single-miRNA hypotheses in the middle GABD window

| | AUC | | | Significance | | |
|---|---|---|---|---|---|---|
| miRNA | AUC | lo 95% CI | hi 95% CI | Wilcoxon | T-test | Chi-square |
| hsa.miR.23a.3p | 0.776 | 0.662 | 0.890 | 5.7E−04 | 3.7E−04 | 1.5E−03 |
| hsa.miR.4732.5p | 0.684 | 0.533 | 0.835 | 2.2E−02 | 3.8E−03 | 1.6E−03 |
| hsa.miR.122.5p | 0.745 | 0.600 | 0.891 | 2.2E−03 | 1.1E−02 | 2.6E−03 |
| hsa.miR.191.5p | 0.722 | 0.587 | 0.857 | 5.6E−03 | 4.8E−03 | 3.9E−03 |
| hsa.miR.326 | 0.696 | 0.555 | 0.838 | 1.4E−02 | 1.1E−02 | 7.8E−03 |
| hsa.miR.941 | 0.698 | 0.561 | 0.835 | 1.4E−02 | 7.2E−03 | 9.8E−03 |
| hsa.miR.223.3p | 0.701 | 0.563 | 0.840 | 1.2E−02 | 1.1E−02 | 9.9E−03 |
| hsa.miR.374b.5p | 0.651 | 0.485 | 0.817 | 5.9E−02 | 3.0E−02 | 1.2E−02 |
| hsa.miR.324.3p | 0.701 | 0.563 | 0.839 | 1.2E−02 | 9.8E−03 | 1.3E−02 |
| hsa.miR.30c.5p | 0.632 | 0.467 | 0.796 | 1.0E−01 | 1.0E−01 | 2.0E−02 |
| hsa.miR.148a.3p | 0.672 | 0.524 | 0.820 | 3.2E−02 | 3.0E−02 | 3.7E−02 |

TABLE 6
Selected single-miRNA hypotheses in the late GABD window

| | AUC | | | Significance | | |
|---|---|---|---|---|---|---|
| miRNA | AUC | lo 95% CI | hi 95% CI | Wilcoxon | T-test | Chi-square |
| hsa.miR.155.5p | 0.715 | 0.603 | 0.827 | 7.1E−04 | 1.3E−03 | 7.7E−04 |
| hsa.miR.30c.5p | 0.705 | 0.590 | 0.820 | 1.2E−03 | 1.3E−02 | 8.4E−04 |
| hsa.miR.1301.3p | 0.640 | 0.519 | 0.761 | 2.7E−02 | 2.2E−02 | 4.4E−03 |
| hsa.miR.23a.3p | 0.688 | 0.575 | 0.801 | 3.1E−03 | 2.1E−02 | 5.3E−03 |
| hsa.miR.10a.5p | 0.671 | 0.549 | 0.793 | 7.1E−03 | 1.0E−02 | 5.7E−03 |
| hsa.miR.485.5p | 0.687 | 0.571 | 0.804 | 3.2E−03 | 9.6E−03 | 7.2E−03 |
| hsa.miR.4446.3p | 0.651 | 0.528 | 0.775 | 1.7E−02 | 1.2E−02 | 9.5E−03 |
| hsa.miR.375 | 0.641 | 0.521 | 0.761 | 2.7E−02 | 1.9E−02 | 1.5E−02 |
| hsa.miR.6842.3p | 0.624 | 0.506 | 0.742 | 5.1E−02 | 5.0E−02 | 1.7E−02 |
| hsa.miR.184 | 0.645 | 0.525 | 0.766 | 2.2E−02 | 2.3E−02 | 2.1E−02 |
| hsa.miR.18a.3p | 0.607 | 0.485 | 0.730 | 9.2E−02 | 4.0E−02 | 2.3E−02 |
| hsa.miR.6747.3p | 0.661 | 0.538 | 0.784 | 1.1E−02 | 5.1E−02 | 2.9E−02 |
| hsa.miR.664a.5p | 0.613 | 0.494 | 0.732 | 7.5E−02 | 3.9E−02 | 3.3E−02 |
| hsa.miR.345.5p | 0.614 | 0.492 | 0.736 | 7.2E−02 | 3.7E−02 | 3.4E−02 |
| hsa.miR.1260b | 0.616 | 0.498 | 0.734 | 6.9E−02 | 4.4E−02 | 3.6E−02 |
| hsa.miR.516b.5p | 0.591 | 0.469 | 0.713 | 1.5E−01 | 6.1E−02 | 3.7E−02 |
| hsa.miR.374b.5p | 0.606 | 0.475 | 0.736 | 9.7E−02 | 5.1E−02 | 3.8E−02 |
| hsa.miR.1273h.3p | 0.624 | 0.506 | 0.742 | 5.1E−02 | 3.6E−02 | 4.0E−02 |
| hsa.miR.99b.3p | 0.628 | 0.508 | 0.749 | 4.3E−02 | 4.3E−02 | 4.3E−02 |
| hsa.miR.409.3p | 0.616 | 0.496 | 0.736 | 6.7E−02 | 4.5E−02 | 4.4E−02 |
| hsa.miR.331.3p | 0.621 | 0.501 | 0.742 | 5.6E−02 | 5.9E−02 | 4.7E−02 |

TABLE 7
Selected miRNA-reversal hypotheses in the entire GABD window

| | | | | Mean | 25% Ie | Mean log |
|---|---|---|---|---|---|---|
| | | Mean | 25% Ie | R\hat{ }2 vs. | R\hat{ }2 vs. | case- |
| Reversal | Rank | AUC | AUC | PE | PE | control |
| hsa.miR.7.5p/hsa.miR.485.5p | 1 | 0.693 | 0.662 | 0.087 | 0.057 | 0.601 |
| hsa.miR.501.3p/hsa.miR.4446.3p | 2 | 0.693 | 0.661 | 0.091 | 0.063 | 0.595 |
| hsa.miR.140.3p/hsa.miR.485.5p | 3 | 0.673 | 0.642 | 0.072 | 0.044 | 0.825 |
| hsa.miR.181a.5p/hsa.miR.130b.5p | 4 | 0.675 | 0.643 | 0.039 | 0.029 | 1.649 |
| hsa.miR.484/hsa.miR.485.5p | 5 | 0.678 | 0.646 | 0.074 | 0.043 | 0.571 |
| hsa.mir.320b.2/hsa.miR.130b.5p | 6 | 0.690 | 0.655 | 0.039 | 0.026 | 0.751 |
| hsa.miR.501.3p/hsa.miR.485.5p | 7 | 0.677 | 0.648 | 0.067 | 0.042 | 0.582 |
| hsa.miR.100.5p/hsa.miR.485.5p | 8 | 0.670 | 0.638 | 0.068 | 0.042 | 0.725 |
| hsa.miR.27a.3p/hsa.miR.485.5p | 9 | 0.674 | 0.644 | 0.070 | 0.046 | 0.497 |
| hsa.miR.451a/hsa.miR.130b.5p | 10 | 0.666 | 0.637 | 0.035 | 0.026 | 3.142 |
| hsa.miR.7.5p/hsa.miR.4446.3p | 11 | 0.690 | 0.659 | 0.076 | 0.037 | 0.436 |
| hsa.miR.182.5p/hsa.miR.485.5p | 12 | 0.665 | 0.634 | 0.063 | 0.036 | 0.726 |
| hsa.miR.425.5p/hsa.miR.130b.5p | 13 | 0.665 | 0.634 | 0.037 | 0.028 | 1.766 |
| hsa.miR.363.3p/hsa.miR.130b.5p | 14 | 0.677 | 0.644 | 0.034 | 0.025 | 1.732 |
| hsa.miR.140.3p/hsa.miR.4446.3p | 15 | 0.672 | 0.642 | 0.052 | 0.023 | 0.600 |
| hsa.miR.320b/hsa.miR.130b.5p | 16 | 0.663 | 0.631 | 0.039 | 0.027 | 1.898 |
| hsa.let.7b.5p/hsa.miR.130b.5p | 17 | 0.663 | 0.632 | 0.035 | 0.026 | 3.043 |
| hsa.miR.134.5p/hsa.miR.130b.5p | 18 | 0.663 | 0.635 | 0.037 | 0.026 | 1.355 |
| hsa.miR.125a.5p/hsa.miR.130b.5p | 19 | 0.661 | 0.630 | 0.037 | 0.028 | 2.170 |
| hsa.miR.125b.5p/hsa.miR.130b.5p | 20 | 0.662 | 0.630 | 0.037 | 0.027 | 1.683 |
| hsa.miR.182.5p/hsa.miR.4446.3p | 21 | 0.679 | 0.650 | 0.044 | 0.014 | 0.453 |
| hsa.miR.181a.5p/hsa.miR.223.5p | 22 | 0.673 | 0.642 | 0.031 | 0.019 | 0.754 |
| hsa.miR.378g/hsa.miR.485.5p | 23 | 0.667 | 0.635 | 0.065 | 0.038 | 0.370 |
| hsa.let.7i.5p/hsa.miR.130b.5p | 24 | 0.662 | 0.629 | 0.035 | 0.026 | 2.643 |
| hsa.miR.127.3p/hsa.miR.485.5p | 25 | 0.662 | 0.627 | 0.056 | 0.029 | 0.523 |
| hsa.miR.363.3p/hsa.miR.485.5p | 26 | 0.699 | 0.666 | 0.040 | 0.011 | 0.369 |
| hsa.miR.140.5p/hsa.miR.379.5p | 27 | 0.665 | 0.637 | 0.034 | 0.007 | 0.619 |
| hsa.miR.125b.5p/hsa.miR.485.5p | 28 | 0.662 | 0.631 | 0.046 | 0.016 | 0.461 |
| hsa.miR.451a/hsa.miR.223.5p | 29 | 0.664 | 0.630 | 0.030 | 0.017 | 1.113 |
| hsa.miR.484/hsa.miR.4446.3p | 30 | 0.662 | 0.630 | 0.049 | 0.017 | 0.429 |
| hsa.miR.25.3p/hsa.miR.130b.5p | 31 | 0.660 | 0.627 | 0.033 | 0.025 | 2.821 |
| hsa.miR.98.5p/hsa.miR.485.5p | 32 | 0.679 | 0.646 | 0.042 | 0.006 | 0.368 |
| hsa.miR.181a.5p/hsa.miR.485.5p | 33 | 0.677 | 0.643 | 0.036 | 0.006 | 0.377 |
| hsa.miR.199a.3p hsa.miR.199b.3p/ | 34 | 0.667 | 0.636 | 0.043 | 0.013 | 0.364 |
| hsa.miR.4446.3p | | | | | | |
| hsa.miR.181a.5p/hsa.miR.199a.5p | 35 | 0.664 | 0.632 | 0.026 | 0.012 | 1.351 |
| hsa.miR.7.5p/hsa.let.7c.5p | 36 | 0.685 | 0.651 | 0.023 | 0.011 | 0.731 |
| hsa.miR.23b.5p/hsa.miR.2110 | 37 | 0.659 | 0.628 | 0.074 | 0.053 | 0.383 |
| hsa.miR.320a/hsa.miR.130b.5p | 38 | 0.657 | 0.626 | 0.035 | 0.026 | 2.743 |
| hsa.miR.451a/hsa.miR.485.5p | 39 | 0.672 | 0.643 | 0.031 | 0.003 | 0.484 |
| hsa.miR.186.5p/hsa.miR.485.5p | 40 | 0.660 | 0.629 | 0.041 | 0.013 | 0.377 |
| hsa.miR.181a.5p/hsa.miR.941 | 41 | 0.659 | 0.625 | 0.031 | 0.013 | 23.965 |
| hsa.miR.134.5p/hsa.miR.485.5p | 42 | 0.666 | 0.634 | 0.035 | 0.006 | 0.358 |
| hsa.let.7b.5p/hsa.miR.485.5p | 43 | 0.669 | 0.636 | 0.030 | 0.003 | 0.480 |
| hsa.miR.140.5p/hsa.miR.486.3p | 44 | 0.658 | 0.629 | 0.035 | 0.014 | 0.492 |
| hsa.miR.3615/hsa.miR.130b.5p | 45 | 0.657 | 0.625 | 0.037 | 0.027 | 1.957 |
| hsa.miR.142.5p/hsa.miR.130b.5p | 46 | 0.657 | 0.627 | 0.029 | 0.020 | 1.496 |
| hsa.miR.363.3p/hsa.let.7c.5p | 47 | 0.679 | 0.645 | 0.021 | 0.010 | 0.714 |
| hsa.miR.330.5p/hsa.miR.654.5p | 48 | 0.660 | 0.624 | 0.040 | 0.021 | 0.689 |
| hsa.miR.1307.3p/hsa.miR.130b.5p | 49 | 0.656 | 0.625 | 0.035 | 0.026 | 2.067 |
| hsa.miR.26b.5p/hsa.miR.485.5p | 50 | 0.672 | 0.639 | 0.036 | 0.003 | 0.365 |

TABLE 8
Selected miRNA-reversal hypotheses in the early GABD window

| | | | | Mean | 25% Ie | Mean log |
|---|---|---|---|---|---|---|
| | | Mean | 25% Ie | R\hat{ }2 vs. | R\hat{ }2 vs. | case- |
| Reversal | Rank | AUC | AUC | PE | PE | control |
| hsa.miR.1224.5p/hsa.miR.433.3p | 1 | 0.765 | 0.725 | 0.179 | 0.139 | 0.937 |
| hsa.miR.125a.3p/hsa.miR.3173.5p | 2 | 0.754 | 0.712 | 0.154 | 0.109 | 1.184 |
| hsa.miR.4732.3p/hsa.miR.381.3p | 3 | 0.816 | 0.774 | 0.139 | 0.062 | 5.249 |
| hsa.miR.4732.3p/hsa.miR.941 | 4 | 0.808 | 0.764 | 0.145 | 0.058 | 5.985 |
| hsa.miR.324.3p/hsa.miR.942.5p | 5 | 0.765 | 0.718 | 0.129 | 0.082 | 1.282 |
| hsa.miR.4433b.3p/hsa.miR.7976 | 6 | 0.755 | 0.712 | 0.133 | 0.087 | 1.191 |
| hsa.miR.370.3p/hsa.miR.193b.5p | 7 | 0.760 | 0.700 | 0.220 | 0.166 | 0.620 |
| hsa.miR.1224.5p/hsa.miR.221.5p | 8 | 0.751 | 0.704 | 0.168 | 0.125 | 0.684 |
| hsa.miR.652.3p/hsa.miR.550a.3.5p | 9 | 0.767 | 0.724 | 0.146 | 0.111 | 0.589 |
| hsa.miR.5189.5p/hsa.miR.374b.5p | 10 | 0.742 | 0.702 | 0.171 | 0.129 | 0.620 |
| hsa.miR.7706/hsa.miR.193b.5p | 11 | 0.827 | 0.779 | 0.191 | 0.145 | 0.451 |
| hsa.miR.652.3p/hsa.miR.941 | 12 | 0.755 | 0.714 | 0.121 | 0.075 | 1.130 |
| hsa.miR.20a.5p/hsa.miR.3173.5p | 13 | 0.782 | 0.733 | 0.128 | 0.064 | 1.049 |
| hsa.miR.155.5p/hsa.miR.3173.5p | 14 | 0.768 | 0.727 | 0.137 | 0.061 | 0.903 |
| hsa.miR.1292.5p/hsa.miR.221.5p | 15 | 0.740 | 0.689 | 0.135 | 0.087 | 0.742 |
| hsa.miR.19b.3p/hsa.miR.760 | 16 | 0.745 | 0.703 | 0.165 | 0.105 | 0.507 |
| hsa.miR.7.5p/hsa.miR.941 | 17 | 0.781 | 0.729 | 0.109 | 0.056 | 9.424 |
| hsa.miR.330.5p/hsa.miR.942.5p | 18 | 0.725 | 0.687 | 0.126 | 0.072 | 2.103 |
| hsa.miR.1976/hsa.miR.505.5p | 19 | 0.720 | 0.671 | 0.154 | 0.102 | 1.018 |
| hsa.miR.550a.3.5p hsa.miR.550a.5p/ | 20 | 0.791 | 0.744 | 0.187 | 0.131 | 0.338 |
| hsa.miR.193b.5p | | | | | | |
| hsa.miR.4433b.3p/hsa.miR.378i | 21 | 0.752 | 0.697 | 0.125 | 0.066 | 0.861 |
| hsa.miR.30a.3p/hsa.miR.181b.5p | 22 | 0.799 | 0.754 | 0.162 | 0.114 | 0.343 |
| hsa.miR.150.3p/hsa.miR.3173.5p | 23 | 0.745 | 0.700 | 0.129 | 0.062 | 0.940 |
| hsa.miR.16.2.3p/hsa.miR.941 | 24 | 0.755 | 0.700 | 0.112 | 0.063 | 1.561 |
| hsa.miR.652.3p/hsa.miR.381.3p | 25 | 0.743 | 0.695 | 0.119 | 0.069 | 0.995 |
| hsa.miR.125a.3p/hsa.miR.381.3p | 26 | 0.734 | 0.684 | 0.113 | 0.069 | 2.507 |
| hsa.miR.382.5p/hsa.miR.221.5p | 27 | 0.725 | 0.680 | 0.136 | 0.094 | 0.751 |
| hsa.miR.500a.3p/hsa.miR.146b.3p | 28 | 0.752 | 0.710 | 0.111 | 0.079 | 0.745 |
| hsa.miR.186.5p/hsa.miR.941 | 29 | 0.767 | 0.715 | 0.103 | 0.056 | 9.304 |
| hsa.miR.6741.5p/hsa.miR.221.5p | 30 | 0.723 | 0.669 | 0.168 | 0.121 | 0.659 |
| hsa.miR.106b.5p/hsa.miR.193b.5p | 31 | 0.768 | 0.714 | 0.240 | 0.180 | 0.304 |
| hsa.miR.1249.3p/hsa.miR.204.5p | 32 | 0.726 | 0.682 | 0.174 | 0.123 | 0.483 |
| hsa.miR.2110/hsa.miR.181b.5p | 33 | 0.761 | 0.710 | 0.164 | 0.105 | 0.328 |
| hsa.miR.144.3p/hsa.miR.942.5p | 34 | 0.742 | 0.682 | 0.120 | 0.071 | 0.799 |
| hsa.miR.885.5p/hsa.miR.146b.3p | 35 | 0.744 | 0.687 | 0.104 | 0.072 | 1.645 |
| hsa.miR.345.5p/hsa.miR.877.5p | 36 | 0.739 | 0.689 | 0.150 | 0.105 | 0.395 |
| hsa.miR.1976/hsa.miR.378e | 37 | 0.770 | 0.717 | 0.191 | 0.117 | 0.287 |
| hsa.miR.345.5p/hsa.miR.200c.3p | 38 | 0.753 | 0.705 | 0.126 | 0.081 | 0.447 |
| hsa.miR.378e/hsa.miR.221.5p | 39 | 0.738 | 0.694 | 0.149 | 0.103 | 0.373 |
| hsa.miR.485.3p/hsa.miR.381.3p | 40 | 0.732 | 0.672 | 0.115 | 0.068 | 1.254 |
| hsa.miR.3182/hsa.miR.518e.5p | 41 | 0.759 | 0.714 | 0.136 | 0.073 | 0.401 |
| hsa.miR.519a.5p hsa.miR.519b.5p | | | | | | |
| hsa.miR.519c.5p hsa.miR.522.5p | | | | | | |
| hsa.miR.523.5p | | | | | | |
| hsa.miR.4433b.3p/hsa.miR.93.3p | 42 | 0.794 | 0.749 | 0.095 | 0.061 | 7.573 |
| hsa.miR.550a.3.5p/hsa.miR.361.5p | 43 | 0.714 | 0.667 | 0.138 | 0.098 | 0.968 |
| hsa.miR.155.5p/hsa.miR.221.5p | 44 | 0.728 | 0.680 | 0.121 | 0.077 | 0.728 |
| hsa.miR.345.5p/hsa.miR.766.5p | 45 | 0.751 | 0.707 | 0.175 | 0.129 | 0.291 |
| hsa.miR.1273h.3p/hsa.miR.3173.5p | 46 | 0.821 | 0.779 | 0.124 | 0.051 | 0.765 |
| hsa.miR.1224.5p/hsa.miR.342.3p | 47 | 0.746 | 0.707 | 0.109 | 0.058 | 1.099 |
| hsa.miR.182.5p/hsa.miR.941 | 48 | 0.752 | 0.709 | 0.099 | 0.058 | 6.410 |
| hsa.miR.320c/hsa.miR.941 | 49 | 0.722 | 0.671 | 0.123 | 0.085 | 0.747 |
| hsa.miR.6852.5p/hsa.miR.505.5p | 50 | 0.713 | 0.667 | 0.145 | 0.090 | 0.785 |

TABLE 9
Selected miRNA-reversal hypotheses in the middle GABD window

| | | | | Mean | 25% Ie | Mean log |
|---|---|---|---|---|---|---|
| | | Mean | 25% Ie | R\hat{ }2 vs. | R\hat{ }2 vs. | case- |
| Reversal | Rank | AUC | AUC | PE | PE | control |
| hsa.miR.877.5p/hsa.miR.24.2.5p | 1 | 0.789 | 0.756 | 0.154 | 0.118 | 0.463 |
| hsa.miR.92b.3p/hsa.miR.24.2.5p | 2 | 0.764 | 0.722 | 0.129 | 0.079 | 0.713 |
| hsa.miR.1299/hsa.miR.433.3p | 3 | 0.718 | 0.677 | 0.126 | 0.085 | 0.663 |
| hsa.miR.1224.5p/hsa.miR.15b.5p | 4 | 0.719 | 0.667 | 0.116 | 0.068 | 0.795 |
| hsa.miR.18a.3p/hsa.miR.375 | 5 | 0.719 | 0.676 | 0.139 | 0.107 | 0.361 |
| hsa.miR.150.3p/hsa.miR.589.5p | 6 | 0.740 | 0.693 | 0.122 | 0.082 | 0.335 |
| hsa.miR.885.5p/hsa.miR.885.3p | 7 | 0.693 | 0.652 | 0.146 | 0.089 | 1.969 |
| hsa.miR.206/hsa.miR.654.3p | 8 | 0.697 | 0.651 | 0.162 | 0.116 | 0.756 |
| hsa.miR.210.3p/hsa.miR.654.3p | 9 | 0.706 | 0.663 | 0.123 | 0.087 | 0.500 |
| hsa.miR.532.3p/hsa.miR.374a.5p | 10 | 0.724 | 0.681 | 0.099 | 0.072 | 0.539 |
| hsa.miR.18a.3p/hsa.miR.942.5p | 11 | 0.754 | 0.707 | 0.129 | 0.074 | 0.250 |
| hsa.miR.206/hsa.miR.24.2.5p | 12 | 0.715 | 0.660 | 0.111 | 0.074 | 0.597 |
| hsa.miR.340.3p/hsa.miR.221.5p | 13 | 0.754 | 0.712 | 0.100 | 0.066 | 0.390 |
| hsa.miR.181a.2.3p/hsa.miR.374a.5p | 14 | 0.698 | 0.657 | 0.106 | 0.074 | 1.214 |
| hsa.miR.92b.3p/hsa.miR.589.5p | 15 | 0.721 | 0.683 | 0.110 | 0.067 | 0.405 |
| hsa.mir.320a/hsa.miR.543 | 16 | 0.758 | 0.717 | 0.116 | 0.075 | 0.244 |
| hsa.miR.30a.3p/hsa.miR.654.3p | 17 | 0.695 | 0.647 | 0.158 | 0.115 | 0.514 |
| hsa.miR.4732.5p/hsa.miR.485.5p | 18 | 0.742 | 0.703 | 0.155 | 0.118 | 0.157 |
| hsa.miR.1285.3p/hsa.mir.378c | 19 | 0.697 | 0.665 | 0.097 | 0.071 | 0.722 |
| hsa.miR.150.3p/hsa.miR.193b.5p | 20 | 0.730 | 0.687 | 0.102 | 0.057 | 0.348 |
| hsa.miR.125b.5p/hsa.miR.543 | 21 | 0.709 | 0.661 | 0.104 | 0.065 | 0.504 |
| hsa.miR.885.5p/hsa.miR.375 | 22 | 0.712 | 0.668 | 0.108 | 0.066 | 0.360 |
| hsa.miR.1285.3p/hsa.miR.326 | 23 | 0.717 | 0.677 | 0.135 | 0.087 | 0.208 |
| hsa.mir.320a/hsa.miR.24.2.5p | 24 | 0.764 | 0.727 | 0.097 | 0.051 | 0.350 |
| hsa.mir.320a/hsa.miR.654.3p | 25 | 0.700 | 0.651 | 0.140 | 0.084 | 0.352 |
| hsa.miR.20b.5p/hsa.miR.6741.5p | 26 | 0.715 | 0.677 | 0.101 | 0.066 | 0.276 |
| hsa.miR.4732.5p/hsa.miR.199a.5p | 27 | 0.735 | 0.691 | 0.193 | 0.138 | 0.113 |
| hsa.miR.877.5p/hsa.miR.589.5p | 28 | 0.703 | 0.657 | 0.111 | 0.072 | 0.256 |
| hsa.miR.25.5p/hsa.miR.24.2.5p | 29 | 0.709 | 0.661 | 0.090 | 0.058 | 0.427 |
| hsa.miR.150.3p/hsa.miR.518e.5p | 30 | 0.703 | 0.659 | 0.126 | 0.080 | 0.194 |
| hsa.miR.519a.5p hsa.miR.519b.5p | | | | | | |
| hsa.miR.519c.5p hsa.miR.522.5p | | | | | | |
| hsa.miR.523.5p | | | | | | |
| hsa.miR.142.5p/hsa.miR.24.2.5p | 31 | 0.714 | 0.674 | 0.094 | 0.043 | 0.488 |
| hsa.miR.4746.5p/hsa.miR.326 | 32 | 0.701 | 0.653 | 0.098 | 0.066 | 0.361 |
| hsa.miR.4732.5p/hsa.miR.4446.3p | 33 | 0.705 | 0.651 | 0.183 | 0.128 | 0.149 |
| hsa.miR.1285.3p/hsa.miR.6741.5p | 34 | 0.704 | 0.663 | 0.113 | 0.080 | 0.180 |
| hsa.miR.181b.5p/hsa.miR.24.2.5p | 35 | 0.722 | 0.677 | 0.093 | 0.046 | 0.330 |
| hsa.miR.1285.3p/hsa.miR.3614.5p | 36 | 0.729 | 0.689 | 0.124 | 0.081 | 0.123 |
| hsa.miR.4732.5p/hsa.miR.24.2.5p | 37 | 0.723 | 0.675 | 0.094 | 0.044 | 0.345 |
| hsa.miR.1306.3p/hsa.miR.326 | 38 | 0.729 | 0.679 | 0.094 | 0.055 | 0.224 |
| hsa.miR.517a.3p hsa.miR.517b.3p/ | 39 | 0.697 | 0.661 | 0.100 | 0.060 | 0.306 |
| hsa.miR.375 | | | | | | |
| hsa.miR.221.3p/hsa.miR.24.2.5p | 40 | 0.699 | 0.660 | 0.092 | 0.047 | 0.614 |
| hsa.miR.374b.5p/hsa.miR.342.3p | 41 | 0.709 | 0.669 | 0.119 | 0.078 | 0.140 |
| hsa.miR.125a.3p/hsa.miR.589.5p | 42 | 0.699 | 0.664 | 0.090 | 0.066 | 0.238 |
| hsa.miR.4732.5p/hsa.miR.516b.5p | 43 | 0.744 | 0.703 | 0.183 | 0.129 | 0.087 |
| hsa.miR.4732.5p/hsa.miR.23a.3p | 44 | 0.726 | 0.675 | 0.233 | 0.175 | 0.093 |
| hsa.miR.374b.5p/hsa.miR.106b.5p | 45 | 0.708 | 0.667 | 0.079 | 0.054 | 0.372 |
| hsa.miR.4732.5p/hsa.miR.1301.3p | 46 | 0.704 | 0.659 | 0.174 | 0.126 | 0.107 |
| hsa.miR.1246/hsa.miR.24.2.5p | 47 | 0.696 | 0.647 | 0.088 | 0.048 | 0.653 |
| hsa.miR.18a.3p/hsa.miR.19b.3p | 48 | 0.711 | 0.666 | 0.075 | 0.040 | 10.486 |
| hsa.miR.92b.5p/hsa.miR.654.3p | 49 | 0.711 | 0.672 | 0.080 | 0.043 | 0.416 |
| hsa.miR.628.3p/hsa.miR.375 | 50 | 0.687 | 0.642 | 0.112 | 0.071 | 0.288 |

TABLE 10
Selected miRNA-reversal hypotheses in the late GABD window

| | | | | Mean | 25% Ie | Mean log |
|---|---|---|---|---|---|---|
| | | Mean | 25% Ie | R\hat{ }2 vs. | R\hat{ }2 vs. | case- |
| Reversal | Rank | AUC | AUC | PE | PE | control |
| hsa.miR.378g/hsa.miR.3182 | 1 | 0.692 | 0.656 | 0.096 | 0.069 | 1.501 |
| hsa.mir.320a/hsa.miR.130b.5p | 2 | 0.713 | 0.675 | 0.084 | 0.063 | 0.836 |
| hsa.miR.486.5p/hsa.miR.155.5p | 3 | 0.699 | 0.660 | 0.072 | 0.045 | 1.093 |
| hsa.miR.451a/hsa.miR.155.5p | 4 | 0.718 | 0.683 | 0.069 | 0.044 | 0.890 |
| hsa.miR.125a.5p/hsa.miR.155.5p | 5 | 0.712 | 0.675 | 0.075 | 0.038 | 0.846 |
| hsa.let.7i.5p/hsa.miR.155.5p | 6 | 0.735 | 0.700 | 0.068 | 0.043 | 0.846 |
| hsa.mir.320b.2/hsa.miR.130b.5p | 7 | 0.776 | 0.743 | 0.069 | 0.047 | 0.826 |
| hsa.let.7b.5p/hsa.miR.155.5p | 8 | 0.714 | 0.676 | 0.067 | 0.041 | 0.959 |
| hsa.miR.25.3p/hsa.miR.155.5p | 9 | 0.707 | 0.673 | 0.072 | 0.044 | 0.822 |
| hsa.miR.516b.5p/hsa.miR.155.5p | 10 | 0.699 | 0.663 | 0.071 | 0.035 | 1.103 |
| hsa.miR.30d.5p/hsa.miR.155.5p | 11 | 0.709 | 0.671 | 0.065 | 0.039 | 0.826 |
| hsa.miR.345.5p/hsa.miR.324.3p | 12 | 0.679 | 0.640 | 0.086 | 0.053 | 2.425 |
| hsa.miR.330.5p/hsa.miR.92b.5p | 13 | 0.718 | 0.677 | 0.135 | 0.093 | 0.555 |
| hsa.miR.320a/hsa.miR.155.5p | 14 | 0.710 | 0.674 | 0.059 | 0.036 | 1.008 |
| hsa.let.7g.5p/hsa.miR.155.5p | 15 | 0.702 | 0.663 | 0.067 | 0.041 | 0.724 |
| hsa.miR.3615/hsa.miR.155.5p | 16 | 0.703 | 0.664 | 0.068 | 0.042 | 0.673 |
| hsa.miR.98.5p/hsa.miR.485.5p | 17 | 0.692 | 0.654 | 0.109 | 0.087 | 0.592 |
| hsa.miR.151a.3p/hsa.miR.155.5p | 18 | 0.712 | 0.671 | 0.061 | 0.036 | 0.799 |
| hsa.miR.221.3p/hsa.miR.155.5p | 19 | 0.689 | 0.652 | 0.070 | 0.040 | 0.779 |
| hsa.miR.127.3p/hsa.miR.485.5p | 20 | 0.701 | 0.664 | 0.074 | 0.042 | 0.564 |
| hsa.let.7i.5p/hsa.miR.485.5p | 21 | 0.697 | 0.658 | 0.073 | 0.032 | 0.701 |
| hsa.miR.423.5p/hsa.miR.155.5p | 22 | 0.696 | 0.659 | 0.056 | 0.032 | 0.987 |
| hsa.miR.1260b/hsa.miR.885.3p | 23 | 0.671 | 0.635 | 0.067 | 0.047 | 12.700 |
| hsa.miR.625.3p/hsa.miR.155.5p | 24 | 0.732 | 0.696 | 0.072 | 0.044 | 0.514 |
| hsa.miR.370.3p/hsa.miR.485.5p | 25 | 0.705 | 0.667 | 0.073 | 0.043 | 0.522 |
| hsa.miR.99a.5p/hsa.miR.155.5p | 26 | 0.698 | 0.657 | 0.057 | 0.033 | 0.835 |
| hsa.miR.20a.5p/hsa.miR.485.5p | 27 | 0.704 | 0.668 | 0.087 | 0.062 | 0.483 |
| hsa.miR.146a.5p/hsa.miR.155.5p | 28 | 0.701 | 0.668 | 0.060 | 0.036 | 0.654 |
| hsa.miR.26a.5p/hsa.miR.155.5p | 29 | 0.700 | 0.663 | 0.056 | 0.032 | 0.803 |
| hsa.miR.134.5p/hsa.miR.485.5p | 30 | 0.707 | 0.665 | 0.076 | 0.042 | 0.501 |
| hsa.miR.181a.5p/hsa.miR.155.5p | 31 | 0.704 | 0.670 | 0.058 | 0.034 | 0.660 |
| hsa.miR.26b.5p/hsa.miR.155.5p | 32 | 0.686 | 0.646 | 0.057 | 0.036 | 0.866 |
| hsa.miR.146b.5p/hsa.miR.155.5p | 33 | 0.702 | 0.662 | 0.058 | 0.035 | 0.666 |
| hsa.miR.320b/hsa.miR.130b.5p | 34 | 0.692 | 0.654 | 0.048 | 0.035 | 2.000 |
| hsa.miR.4443/hsa.miR.130b.5p | 35 | 0.688 | 0.653 | 0.048 | 0.033 | 2.620 |
| hsa.miR.181a.5p/hsa.miR.130b.5p | 36 | 0.689 | 0.649 | 0.050 | 0.035 | 1.779 |
| hsa.miR.1323/hsa.miR.485.5p | 37 | 0.706 | 0.668 | 0.091 | 0.052 | 0.448 |
| hsa.miR.126.3p/hsa.miR.155.5p | 38 | 0.701 | 0.662 | 0.060 | 0.035 | 0.587 |
| hsa.miR.26b.5p/hsa.miR.485.5p | 39 | 0.675 | 0.635 | 0.084 | 0.060 | 0.690 |
| hsa.miR.320b/hsa.miR.155.5p | 40 | 0.695 | 0.656 | 0.055 | 0.031 | 0.871 |
| hsa.miR.181a.5p/hsa.miR.485.5p | 41 | 0.698 | 0.658 | 0.077 | 0.037 | 0.513 |
| hsa.miR.425.5p/hsa.miR.155.5p | 42 | 0.699 | 0.659 | 0.067 | 0.041 | 0.523 |
| hsa.let.7b.5p/hsa.miR.485.5p | 43 | 0.694 | 0.657 | 0.070 | 0.026 | 0.782 |
| hsa.miR.320a/hsa.miR.485.5p | 44 | 0.674 | 0.641 | 0.072 | 0.034 | 0.782 |
| hsa.miR.451a/hsa.miR.485.5p | 45 | 0.688 | 0.650 | 0.071 | 0.027 | 0.767 |
| hsa.mir.320a/hsa.miR.485.5p | 46 | 0.699 | 0.662 | 0.094 | 0.062 | 0.446 |
| hsa.miR.185.5p/hsa.miR.485.5p | 47 | 0.680 | 0.642 | 0.071 | 0.034 | 0.672 |
| hsa.miR.363.3p/hsa.miR.155.5p | 48 | 0.701 | 0.665 | 0.063 | 0.038 | 0.524 |
| hsa.miR.4443/hsa.miR.155.5p | 49 | 0.714 | 0.677 | 0.051 | 0.028 | 0.885 |
| hsa.miR.27a.3p/hsa.miR.485.5p | 50 | 0.693 | 0.657 | 0.078 | 0.043 | 0.480 |

TABLE 11
Selected single-miRNA and miRNA-reversal
hypotheses across GABD windows

| miRNA(s) | Window |
|---|---|
| hsa.miR.127.3p/hsa.miR.485.5p | Full |
| hsa.miR.423.3p | Early |
| hsa.miR.516b.5p | Early |
| hsa.miR.4732.3p/hsa.miR.381.3p | Early |
| hsa.miR.4732.3p/hsa.miR.941 | Early |
| hsa.miR.155.5p/hsa.miR.3173.5p | Early |
| hsa.miR.1273h.3p/hsa.miR.3173.5p | Early |
| hsa.miR.155.5p | Late |
| hsa.miR.331.3p | Late |
| hsa.miR.451a/hsa.miR.155.5p | Late |
| hsa.miR.125a.5p/hsa.miR.155.5p | Late |
| hsa.let.7i.5p/hsa.miR.155.5p | Late |
| hsa.let.7b.5p/hsa.miR.155.5p | Late |
| hsa.miR.25.3p/hsa.miR.155.5p | Late |

### Example 2. Analysis of Verified Predictors of Placental Dysfunction

This example describes further analysis of the experiments described in Example 1.

Placental dysfunction, for which the most common clinical manifestations are preeclampsia (PE) and intrauterine growth restriction (IUGR), is an important cause of fetal and maternal morbidity and mortality. Affecting approximately 5% of pregnancies (Bartsch et al., BMJ 353:i1753 (2016)), PE is the second leading cause of maternal mortality (Ness, Am J Obstet Gynecol 175(5):1365-1370 (1996); Sibai, Obstet Gynecol 102(1):181-92 (2003)) and the leading cause of medically indicated preterm birth (miPTB) in the US, accounting for 15% of all PTBs (Sibai, Semin Perinatol 30(1):16-19 (2006)). PE is typically diagnosed by a combination of new-onset hypertension and proteinuria, but severe cases can be associated with maternal end organ damage, including cerebral edema, pulmonary edema, liver or kidney failure, hemolysis, or thrombocytopenia, placental abruption, seizures (eclampsia), or maternal and fetal death. The clinical manifestations of PE become apparent in the second half of pregnancy, but they arise from dysregulation of feto-placental development and/or maternal adaptation to pregnancy in early pregnancy. Low-dose aspirin therapy started between 12-28 weeks of gestation has been shown to decrease the risk of PE and IUGR in pregnancies with preexisting hypertension, preexisting diabetes, multifetal gestation, renal disease, autoimmune disease, and preeclampsia with an adverse pregnancy outcome in a prior pregnancy. For this reason, the US Preventative Task Force (USPTF) has recommended prophylactic low-dose aspirin in pregnancies with these clinical risk factors for PE (Henderson et al., in Low-Dose Aspirin for the Prevention of Morbidity and Mortality From Preeclampsia: A Systematic Evidence Review for the U.S. Preventive Services Task Force, Rockville (Md.) (2014)). However, the majority of patients who develop preeclampsia or IUGR do not have known risk factors, and thus it is an immediate priority to discover other methods for identification of high-risk pregnancies and to determine whether they would benefit from aspirin prophylaxis.

Early identification of pregnancies that have an elevated risk for developing PE would provide for customization of prenatal care to incorporate the appropriate intensity of surveillance. It would also allow for selective enrollment of high-risk pregnancies for clinical trials on novel agents for prevention or treatment of PE. However, current strategies for early prediction of PE are limited by either suboptimal performance and/or clinical feasibility. Current modalities for first and second trimester risk assessment involve the assessment of maternal characteristics, measurement of specific analytes in the maternal blood, and sonographic measurement of the uterine artery pulsatility index (Poon et al., Fetal Diagn Ther 33(1):16-27 (2013); Yliniemi et al., Clin Med Insights Reprod Health 9:13-20 (2015)). The highest performing first trimester risk assessment algorithm was based on a multivariate model incorporating a variety of maternal characteristics (e.g., maternal age, weight, height, race, smoking, assisted reproductive technologies, prior pregnancy with preeclampsia or small for gestational age (<10th percentile, SGA), chronic hypertension, diabetes mellitus, lupus, antiphospholipid syndrome, and family history of preeclampsia.), serum analyte values** (e.g., pregnancy-associated plasma protein A (PAPP-A) and Placental Growth Factor (PLGF)), mean arterial pressure, and uterine artery pulsatility index, and reported detection rates of 95.3% for early (<34 week) PE, 45.6% for late PE, 55.5% for preterm SGA, and 44.3% for term SGA with a false positive rate (FPR) of 10% (Poon et al., Fetal Diagn Ther 33(1):16-27 (2013)). Vascular endothelial growth factor (VEGF), soluble fms-like tyrosine kinase 1 (sFlt-1), and PLGF levels have shown promise as predictive biomarkers in the third trimester, primarily due to their high negative predictive value (Levine et al., New England Journal of Medicine 350(7):672-683 (2004); Tjwa et al., Cell and Tissue Research 314(1):5-14 (2003); Caillon et al., Ann Lab Med 38(2):95-101 (2018)).

Over the past decade, extracellular RNAs (exRNAs) in a variety of biofluids have been shown to have potential value as diagnostic and prognostic biomarkers for a variety of conditions, including cancer, heart disease, neurodegenerative disease, and liver injury (reviewed in (Das et al., Cell 177(2):231-242 (2019)). As described below, studies were performed to build on observations that there is exRNA of feto-placental origin in the maternal circulation (Ng et al., Proc Natl Acad Sci USA 100(8):4748-4753 (2003); Ge et al., Prenat Diagn, 25(10):912-918 (2005); Go et al., Clin Chem 50(8):1413-1414 (2004); Tsui et al., J Med Genet 41(6):461-467 (2004); Poon et al., Clin Chem 46(11):1832-1834 (2000)), suggesting that exRNAs can serve as biomarkers that can be utilized in non-invasive interrogation of placental function. Extracellular miRNA biomarkers associated with PE have been described previously (Gan et al., Medicine (Baltimore) 96(28):e7515 (2017); Gunel et al., Placenta 52:77-85 (2017); Hromadnikova et al., PLoS One 12(2):e0171756 (2017); Hromadnikova et al., Mediators Inflamm 2013:186041 (2013); Jairajpuri et al., Gene 627:543-548 (2017); Li et al., Biomed Res Int 2013:970265 (2013); Luque et al., Sci Rep 4:4882 (2014); Martinez-Fierro et al., Arch Gynecol Obstet, 297(2):365-371 (2018); Miura et al., J Obstet Gynaecol Res, 41(10):1526-1532 (2015); Motawi et al., Arch Biochem Biophys 659:13-21 (2018); Salomon et al., J Clin Endocrinol Metab 102(9):3182-3194 (2017); Stubert et al., Hypertens Pregnancy 33(2):215-235 (2014); Timofeeva et al., Placenta 61:61-71 (2018); Ura et al., Taiwan J Obstet Gynecol 53(2):232-234 (2014); Wu et al., Reproduction 143(3):389-397 (2012); Xu et al., Hypertension 63(6):1276-1284 (2014); Yang et al., Clin Chim Acta 412(23-24):2167-2173 (2011); Yang et al., Mol Med Rep, 12(1):527-534 (2015); Yoffe et al., Sci Rep 8(1):3401 (2018)). Importantly, only two previous studies verified their initial findings in an independent cohort. One of these studies collected all samples after diagnosis, with the small Discovery cohort (8 cases and 4 controls) being analyzed by small RNAseq and the Verification cohort (38 cases and 32 controls) being analyzed by qRTPCR (Li et al., Biomed Res Int 2013:970265 (2013)). In the other study, the Discovery cohort samples (28 cases and 26 controls) were collected after diagnosis and analyzed by small RNAseq and the Verification cohort samples (only 6 cases and 10 controls) were collected pre-symptomatically and analyzed by qRTPCR (Timofeeva et al., Placenta 61:61-71 (2018)).

These experiments were aimed at discovery and verification of extracellular miRNA predictors for PE. Cases and controls were selected from two studies in which maternal serum was collected from asymptomatic women between 17-28 weeks gestation and clinical outcomes were assessed after delivery. Cases and controls were divided into adequately sized Discovery (49 cases and 92 controls) and Verification (24 cases and 47 controls) cohorts. Small RNA sequencing was used for biomarker Discovery and Verification, and univariate (single miRNA) and bivariate (ratios of pairs of miRNAs, also termed reversals) biomarkers were investigated. Key aspects of the study design were that Discovery and Verification were performed on independent sets of subjects, and that the investigators who developed univariate and bivariate models were blinded to the clinical outcomes of subjects in the Verification set. Candidate models were locked before Verification analysis.

As described below in more detail, small RNA-seq of maternal serum exRNAs was performed to discover and verify miRNAs differentially expressed in patients who later developed preeclampsia. Serum collected from 73 preeclampsia cases and 139 controls between 17-28 weeks gestational age (GA), divided into separate Discovery and Verification cohorts, was analyzed by small RNA seq. Discovery and verification of univariate and bivariate miRNA biomarkers revealed that bivariate biomarkers verified at a markedly higher rate than univariate biomarkers. The majority of verified biomarkers contained miR-155-5p, which has been reported to mediate the preeclampsia-associated repression of eNOS by TNFα. Deconvolution analysis revealed that several verified miRNA biomarkers came from the placenta and were likely carried by placenta-specific extracellular vesicles. Both univariate extracellular miRNAs biomarkers and bivariate reversals were discovered and verified that identified asymptomatic patients at elevated risk for later development of preeclampsia. The verification rate for reversals was markedly higher than for univariate biomarkers, indicating that the use of reversals can confer a degree of internal normalization that increases robustness.

Experimental Model and Subject Details

Human Subjects

Research on human samples were conducted following written informed consent under Institutional Review Board (IRB) protocols approved by the Human Research Protections Program at UCSD. Biofluid and RNA samples were labeled with study identifiers; no personally identifiable information was shared among participating laboratories.

Study Subject Enrollment

Maternal serum was collected between 17-28 weeks gestation. Samples were obtained from the high-risk Placental Study at the University of California, San Diego and from the average-risk Proteomic Assessment of Preterm Risk (PAPR) Study at Sera Prognostics. Eligibility criteria for the two studies are listed in Tables 12A and 12B. Eligibility criteria for the UCSD Placenta Study included: abnormal first or second trimester analytes defined by PAPP-A<0.3 multiples of the medium (MoM), alpha fetoprotein (AFP)>2.5 MoM, Inhibin>2.0 MoM, and Estradiol<0.30 MoM and/or prior adverse pregnancy outcome attributable to preeclampsia and/or maternal co-morbidities associated with increased risk for preeclampsia.

Clinical Data Collection, Adjudication of Pregnancy Outcome, and Selection of Cases and Controls for Analysis

After delivery, relevant clinical data were abstracted from the Clarity Clinical Data Warehouse, which houses clinical data exported from UCSD's EPIC Electronic Medical Record for quality improvement and research uses. These data were then used by adjudicators to determine the clinical outcome for each case, with each case adjudicated by two OB/GYN physicians, at least one of which was Board-Certified in Maternal Fetal Medicine. For the samples from the Sera Prognostics PAPR Study, clinical diagnoses were abstracted by clinical research staff at each participating site from the subjects' medical records. No source document verification or adjudication of diagnoses was performed. From the UCSD Placenta Study, 19 cases and 29 controls were selected. From the Sera PAPR Study, 54 cases and 110 controls were selected.

Method Details

Reagents

Antibodies used in the studies include anti-CD63 antibody (BD Pharmingen; San Jose, Calif.), anti-AGO2 Antibody (Abcam; Cambridge, UK), anti-PLAP Antibody (Abcam), anti-CD63 Antibody (BD Pharmingen), anti-AGO2 Antibody (Abcam), and anti-PLAP Antibody (Abcam). Commercial assays used in the studies include miRNeasy micro kit (Qiagen; Germantown, Md.), MIRVANATM miRNA Isolation Kit, without phenol (ThermoFisher Scientific; Waltham, Mass.), RNA Clean & Concentrator-5 (Zymo Research; Irvine, Calif.), DNA Clean & Concentrator-5 (Zymo Research), QUANTIT™ RIBOGREEN™ RNA Assay Kit (ThermoFisher Scientific), QUANTIT™ PICOGREEN™ dsDNA Assay Kit (ThermoFisher Scientific), Agilent RNA 6000 Pico Kit (Agilent Technologies; Santa Clara, Calif.), Agilent RNA 6000 Nano Kit (Agilent Technologies), Bioanalyzer High Sensitivity DNA Analysis (Agilent Technologies), and NEBNext Small RNA Library Prep Set for Illumina (Multiplex Compatible) (New England Biolabs; Ipswich, Mass.). Deposited data includes small RNA-seq data and miRNA. Software and algorithms used include exceRpt small RNA-seq pipeline for exRNA profiling (Genboree Bioinformatics, genboree.org/java-bin/login.jsp).

Maternal Serum:

Maternal blood was collected by peripheral venipuncture into BD Vacutainer serum blood collection tubes (Becton Dickinson; Franklin Lakes, N.J.), held at room temperature for at least 10 minutes and centrifuged at 2000×g for 10 minutes. The serum was divided into 1 mL aliquots and stored at -80° C. until RNA extraction was performed.

Placenta Tissue:

Placenta tissue samples (<0.5 cm×0.5 cm×0.5 cm) were collected after elective termination procedures (5-22 weeks gestational age) or delivery (22-42 weeks), and immediately placed in RNAlater (ThermoFisher). After storage in RNAlater for 24 hours-7 days, the tissue samples were transferred into clean microfuge tubes and stored at −80° C. until RNA extraction.

Adult tissue samples (<0.5 cm×0.5 cm×0.5 cm) were collected at the time of organ harvest for organ donation and immediately placed in RNAlater (ThermoFisher). After storage in RNAlater for 24 hours-7 days, the tissue samples were transferred into clean microfuge tubes and stored at -80° C. until RNA extraction.

Blood Cells:

PBMC, Platelets, and RBCs:

Human blood samples were collected with written consent from donors≥18 years of age under an IRB protocol approved by the Human Research Protections Programs at UCSD. Biofluid samples were labeled with study identifiers.

Whole blood was collected from two male and two female healthy non-pregnant adult donors, 22-50 years of age. For each donor, blood was collected in the following order: ˜8 mL into a serum BD Vacutainer collection tube (Becton Dickinson) followed by 3×4.5 mL into CTAD (0.11 M buffered trisodium citrate, 15 M theophylline, 3.7 M adenosine, 0.198 M dipyridamole) collection tubes (Becton Dickinson). The serum tubes were held at room temperature for 20 minutes prior to centrifugation at 2000×g for 5 minutes with no brake. 5004, aliquots were transferred from the clear upper serum layer into screw cap 2 mL centrifuge tubes and frozen at −80° C. until they were processed.

Peripheral blood mononuclear cells (PBMC), platelets, and washed red blood cells (RBC) were purified from the CTAD tubes. Wide bore pipette tips were used at all relevant steps to reduce cell shearing and lysis.

For platelets, the CTAD tubes were centrifuged at 100×g for 20 minutes with no brake and all but ˜100 μL of the supernatant was added to a fresh 15 mL conical centrifuge tube. Freshly prepared Prostaglandin I2 (PGI2) (Abcam) was added to ˜2 μM final concentration. The Platelet Rich Plasma (PRP) was then centrifuged at 100×g for 20 minutes with no brake, and all but ˜100 μL of the supernatant was added to a fresh 15 mL conical centrifuge tube. To pellet the platelets, this tube was centrifuged at 800×g for 20 minutes with no brake. The platelet pellet was washed without pellet resuspension in 10 mL of Platelet Wash Buffer (PWB) (1× wash buffer: 10 mM Tris, pH 7.5, 138 mM NaCl, 1.8 mM CaCl₂, 0.49 mM MgCl₂, 1 uM PGI2). The material was centrifuged at 800×g for 10 minutes with no brake and the supernatant material was removed to near completion. The platelet pellet was gently resuspended in 2 mL of PWB and transferred to a 2 mL centrifuge tube. The mixture was centrifuged at 800×g for 10 minutes with no brake, and the supernatant material was removed to near completion. The platelet pellet was stored at −80° C. until processed.

PBMCs and RBCs were purified from the material remaining after the first CTAD tube centrifugation step. For the PBMCs, the remaining PRP, buffy coat, and a small portion of the RBCs were combined by patient and transferred from the CTAD tubes into a fresh 15 mL tube. Sufficient PGI2 was added such that the concentration would be 2 μM when PWB was added to a 10 mL total volume. The material was gently inverted several times to mix and centrifuged at 100×g for 20 minutes with no brake. The supernatant material was removed to near completion and the pellet was mixed in 10 mL of RBC lysis buffer (150 mM NH₄Cl, 10 mM NaHCO₃, 1.27 mM EDTA) placed at room temperature for 20 minutes. The material was centrifuged at 500×g for 5 minutes and the supernatant was discarded. The pellet was washed twice with 10 mL of Dulbecco's phosphate-buffered saline (DPBS) each time and centrifuged as before. The pellet was gently resuspended in 2 mL of DPBS and the material was transferred to a 2 mL centrifuge tube and centrifuged as before. The supernatant was carefully removed and the pellet material in the tube was placed at −80° C. until processed.

The remaining RBCs within the CTAD tubes were combined by patient into a 50 mL conical tube and DPBS was added to 50 mL. The cells were centrifuged at 500×g for 5 minutes with no brake and the supernatant was decanted. This washing process was repeated two more times. 200 μL aliquots of the remaining washed RBC pellet were transferred into 2 mL screw cap tubes and stored at -80° C. until processed.

Lymphocytes and Monocytes:

Human peripheral blood was obtained from healthy adult volunteers in accordance with the guidelines of the Institutional Review Board of Beth Israel Deaconess Medical Center after informed consent was obtained in accordance with the Declaration of Helsinki. Ten milliliters of blood from healthy donors were collected via cubital venipuncture into a syringe prefilled with 2.3 mL of 6% Dextran 500 (Sigma-Aldrich; St. Louis, Mo.) and 1 mL of 3.2% sodium citrate (Sigma-Aldrich). After gentle mixing the blood was sedimented for 45 minutes with the syringe's nozzle up. The RBC-free fraction was washed once by centrifugation at 2000×g for 10 minutes. The resulting pellet was resuspended in 0.5 mL of Hank's Balanced Salt Solution with Calcium and Magnesium (HBSS++).

The cells were sorted using a Becton Dickinson FACSAria Ilu cell sorter equipped with five lasers (350 nm, 405 nm, 488 nm, 561 nm, and 640 nm). The cell populations were sorted through a 70 μm nozzle tip at a sheath pressure of 70 psi and a drop drive frequency of 90-95 kHz. A highly pure sorting modality (4-way purity sorting for FACS Aria, Masks at 0-32-0) was chosen for cell sorting. The flow rate was maintained at an approximate speed of 10,000 events/second. Lymphocytes and monocytes were gated based on forward-scattered light (FSC) and side-scattered light (SSC) FSC/SSC properties. The FSC values are proportional to the diameter of the interrogated cells, whereas the SSC values provides information about the internal complexity of the interrogated cell or its granularity. Sorted cells were collected in 5 ml polypropylene tubes containing 1 ml collection medium (RPMI supplemented with 50% FBS, 100 μg/ml gentamicin, 4 mM L-glutamine, 20 mM HEPES) and stored at −80° C. until processed.

Immunoprecipitation of exRNA Carriers

Antibody biotinylation: Antibodies raised against CD63, AGO2, and PLAP were used. Sodium azide was removed from antibody stocks using the Zeba spin desalting column (7K MWCO, 0.5 ml, Thermo Fisher Scientific). Antibodies were then biotinylated using the EZ-LINK™ Sulfo-NHS-LC-Biotin reagent (ThermoFisher), following manufacturer's protocol. Briefly, 10 mM biotin solution was prepared by dissolving 1 mg of no-weight Sulfo-NHS-LC-Biotin in 180 μL ultrapure water (purified by Milli-Q Biocel System; MilliporeSigma, Burlington, Mass.). Appropriate volume of biotin was added to antibody in order to gain about 20-fold excess biotin-to-antibody molar ratios. The mixture was incubated at room temperature for 2 hr. The biotinylated antibody was then filtered using another desalting column, and the final concentration of the biotinylated antibody was measured using a NanoDrop UV spectrophotometer (ThermoFisher) based on absorption at 280 nm.

Magnetic bead preparation: DYNABEADS™ MYONE™ Streptavidin T1 (Invitrogen; Carlsbad, Calif.) suspension was transferred to 2.0 ml microcentrifuge tube and placed on the DYNAMAG™-2 magnetic rack followed by aspiration of supernatant. The tube was removed from the magnetic rack and washed with 0.01% Tween-20. The washing step was repeated twice. For blocking purpose, the beads were washed 3 times in PBS containing 0.1% bovine serum albumin (BSA) prior to use.

Immunoprecipitation: The immunoprecipitation procedure was performed by incubating the serum with antibody conjugated beads. Briefly, serum from pregnant females was thawed and diluted 1:1 with double filtered 1× PBS (Pierce™ 20× PBS, ThermoFisher). Every 1,000 μL of serum was invert-mixed with 6 μg biotinylated antibody for 20 min at room temperature (RT) on a HULAMIXER® Sample Mixer (ThermoFisher) at 10 rpm. Then, 390 μL of washed Dynabeads was added to the mixture and invert-mixed for 25 min at RT on a Hula mixer at 10 rpm. The mixture was then washed three times with 0.1% BSA and subjected to RNA extraction.

RNA extraction from Dynabeads: RNA was extracted using the miRNeasy mini kit (Qiagen) following manufacturer's protocol. In brief, the Dynabeads were subjected to phenol/chloroform extraction step for RNA extraction using QIAZOL™ Lysis Reagent (Qiagen) followed by chloroform. The aqueous phase was used as input into the miRNeasy procedure and the RNA was eluted in 14 μL of nuclease-free water. To avoid contamination with genomic DNA, the RNA samples were also treated with deoxyribonuclease I (DNase I, Invitrogen). The quality of RNA was assessed by using the RNA 6000 Nano Pico Kit (Agilent Technologies) and the Bioanalyzer 2100 (Agilent Technologies). The eluted RNA was dried down using a speedvac, and used as input into the small RNAseq library preparation process. Small RNAseq libraries were generated and size selected as described below.

RNA Extraction and Small RNA Sequencing Library Construction

Serum:

RNA was extracted from 500 μL of maternal serum using the miRNeasy Micro kit (Qiagen) according to the manufacturer's protocol with a few modifications. Briefly, 2.5 mL of the QIAzol Lysis Reagent (Qiagen) was added to the serum and incubated for 5 min. To this 500 μL of chloroform was added, incubated for 3 min and centrifuged for 15 min at 12,000×g at 4° C. The RNA in the aqueous phase was precipitated by adding 1.5× volumes of 100% ethanol and then loaded on to MinElute spin column and centrifuged at 1,000×g for 15 s. The columns were then washed with 700 μL Buffer RWT (Qiagen), 500 μL Buffer RPE (Qiagen) and 500 μL 80% ethanol consecutively by centrifuging for 15 s at >8000×g. After a final drying spin at full speed for 5 min, RNA was eluted in 35 μL RNase-free water directly to the center of the spin column membrane and centrifuging for 1 min at 100×g followed by 1 min at full speed. The RNA was then concentrated using the Zymo RNA Clean and Concentrator-5. 60 μL of the RNA binding buffer and 90 μL of 100% ethanol was added to 30 μL of RNA, transferred to the Zymo-Spin IC columns and centrifuged at 2000×g for 30 s. The column was washed with 700 μL and 400 μL of RNA wash buffer and centrifuged at full speed for 30 sec and 2 min, respectively. The RNA was then eluted into a final volume of 9 μL RNase free water. The size distribution and quality of the extracted RNA was verified on Agilent RNA 6000 Pico chips using the Agilent 2100 Bioanalyzer instrument.

Tissues:

RNA was extracted from the placental and adult tissue samples using the miRVANA miRNA Isolation Kit (Ambion) using the manufacturer's Total RNA protocol. 400 μL of frozen (approximately −70° C.) 1 mm silica/zirconia beads (BioSpec Products; Bartlesville, OK) were added to each frozen tissue piece, along with 800 μL RNA lysis solution, and placed into a MINIBEADBEATER (BioSpec Products) for one minute. The resultant material was immediately centrifuged at 17,000×g for 5 minutes. To the supernatant (600 μL), 60 μL miRNA Homogenate Additive was added, vortexed and incubated on ice for 10 min. 600 μL of Acid Phenol was added, vortexed for 30 s and centrifuged for 5 min at max speed. The aqueous phase was transferred to a fresh tube and 1.25 volumes of 100% ethanol was added. The solution was transferred to filter tube, spun at 10,000×g for 30 s and then washed once with 7004, wash solution 1 and 2× with 500 μL wash solution ⅔ at 10,000×g for 30 s. After a drying spin max speed for 2 min, RNA was eluted with 100 μL of 95° C. RNase free water at max speed for 30 s. The extracted RNA was quantified using the RiboGreen reagent (ThermoFisher). The size distribution and quality of the extracted RNA was verified on Agilent RNA 6000 Nano chips using the Agilent 2100 Bioanalyzer instrument.

Blood Cells:

Small RNAseq libraries were prepared from 1.2 μL input RNA using the NEBNext Small RNA Sequencing Library Preparation kit (New England BioLabs), using a mosquito HTS automated nanoliter liquid handler (TTP Labtech). For the automation, the reaction volume was reduced to ⅕th of the manufacturer's recommended volume and the adaptors were diluted to ⅙th of the manufacturer's recommended concentration.

Libraries were then cleaned and concentrated using the Zymo DNA Clean and Concentrator-5 kit (Zymo Research) with a 25 μL elution volume and quantified using the Quant-iT Picogreen DNA Assay High Sensitivity kit (ThermoFisher). The size distributions of the library products were determined using the Agilent High Sensitivity DNA chip on the Agilent 2100 Bioanalyzer instrument. The libraries were then pooled (up to 48 samples/pool) based on their concentrations and their size distribution, to obtain similar numbers of miRNA reads among libraries. The pooled libraries were then subjected to size selection on the PippinPrep instrument to remove unincorporated adapters and primers and adapter-dimers. For exRNA from biofluid samples, including immunoprecipitation experiments, a 115-180 bp size selection window was used and for placental tissues samples, a 120-135 bp window was used.

Small RNA Sequencing

Libraries were sequenced on a HiSeq 4000 (Illumina; San Diego, Calif.) with single-end 75 bp reads.

Quantification and Statistical Analysis

Data Analysis

Clinical Data:

Clinical data were analyzed using Student t-test and Mann-Whitney U Test where appropriate (SPSS version 24; IBM, Armonk, N.Y.).

Biofluid exRNA small RNAseq Data:

Small RNAseq data from the exRNA samples were processed, including adapter trimming and mapping to miRBase (miRbase v.21) to yield Raw Count data, using the ExceRpt small RNA sequencing data analysis pipeline version 4.6.2 with minimum insert length set at 10 nt and no mismatches permitted on the Genboree workbench (genboree.org/theCommons/projects/exrna-tools-may2014/wiki/Small%2ORNA-seq%20Pipeline) (Coarfa et al., BMC Bioinformatics 15(Suppl 7):S2-S2 (2014); Riehle et al., BMC Bioinformatics 13(Suppl 13):S11-S11 (2012); Subramanian et al., Journal of Extracellular Vesicles, 4:27497 (2015)).

The Placental Dysfunction Clinic samples were unblinded and included in the Discovery set. The Sera samples were initially blinded and were divided between Discovery and Verification sets, in a manner that resulted in a similar distribution of gestational age at blood draw (GABD), and of the proportions of preeclampsia cases to non-preeclamptic controls across all GABD and in 1- and 3-week windows of GABD between the Discovery and Verification sets.

Filtering was performed to remove individual miRNAs with >70% missing values. Raw miRNA count data after filtering to remove poorly expressed miRNAs were tabulated. Read counts were log2 transformed. Sample-to-sample normalization was carried out through stabilization of variance and reduction in bias across distributions of read counts. Variance stabilizing transformation and bias reduction are useful for making high- and low- read-count samples and miRs more tractable, as stabilizing variance reduces heteroskedasticity and reducing bias removes sample-wide mean shifts. The PEER package (Sanger Institute; Cambridge, UK) was run to reduce batch effects while retaining biological variation (Astrand, Journal of Computational Biology 10(1):85-102 (2003)). Replicate data was then collapsed to single values. AUCs were generated with the pROC package, using the Delong and bootstrap methods to establish the confidence intervals (CIs) (Robin et al., BMC Bioinformatics 12(1):77 (2011); Stegle et al., PLOS Computational Biology 6(5):e1000770 (2010); Parts et al., PLOS Genetics 7(1):e1001276 (2011)). Analysis was performed using R 3.4.3. Processed miRNA data after normalization and batch correction were tabulated.

Four windows of GABD were considered: full window (119-196 days), early window (119-152), middle window (138-172 days), and late window (156-196 days). Univariate miRNA models were fit to the entire dataset range of gestational age at blood draw (GABD) and to early, middle and late GABD windows.

For the Discovery phase, univariate models with significant chi-square p-values (p-value<0.05) between residual and null deviance were selected for each GABD window (Table 15). Univariate predictors with a chi-squared p-value <0.05 were identified for each GABD window, and verified using a 5th percentile AUC cutoff of 0.5 (Table 15). Univariate models for which the lower confidence interval (CI) area under the curve (AUC) was >0.5 were considered to have passed Verification (Tables 15, 17 and 18).

In Table 17, predictors that were Discovered and Verified in the same GABD window are listed in bold; predictors that were Discovered in one GABD window and Verified in a different window are shown in italics. In Table 18, mean AUC values between 0.6-0.8 were identified. miRNAs for which the Verification Mean AUCs for each GABD Window and averaged across all GABD windows columns are >0.6 are bolded. For single miRNA expression values with significance values, relative expression and Wilcoxon p-values for each miRNA in/control for Discovery and Verification sets was determined. For reversal scores, 25th, 50th, and 75th percentile reversal scores, as well as the median shift in the reversal score, were determined for each reversal in Discovery and Verification. The chromosome on which each miRNA is encoded is indicated. For miRNA cluster, if a given miRNA is located in a miRNA cluster, a “Y” is entered. For tissue atlas, for each miRNA, the estimated percent contribution of each cell and tissue type is listed. The cell or tissue type with the highest percentage, and with percentages within 10 percentage points of the highest percentages, are listed in decreasing abundance in the “Max/(Max-10%)” columns. For associated carrier subclasses, for miRNAs that show differential enrichment in one or more carrier subclasses by immunoprecipitation, the enriched subclass(es) are listed. For extracellular miRNAs that have been previously been associated with preeclampsia, the prior report is indicated.

In Discovery, bivariate reversals (ratios of log of miRNA counts) were ranked by an inverse rank sum using 1000 bootstraps (Ripley, “Stochastic Simulation,” John Wiley & Sons (2009)). For each iteration, the AUC, the squared correlation between the ratio and a 1/0 conversion of the diagnosis column (PE=1, control=0), and the mean difference between cases and controls were calculated. Five ranks were derived from the resulting statistics across the 1000 iterations: 1) the mean of the AUCs of the reversal; 2) the lower 25% quantile of the AUCs of the reversal; 3) the mean of the squared correlation; 4) the lower 25% of the squared correlation; and 5) the square of the differences between the case/control mean shift. Each rank was then inverted and summed for each reversal to obtain the final ranking (Table 16). The top ranked 50 reversals for each GABD window were selected, and verified using a 5th percentile AUC cutoff of 0.5 (Table 16). Reversals were considered to pass Verification if their lower CI did not cross 0.5 in the Verification dataset using the DeLong method for CI calculation (Tables 16-18).

Power analysis examined the power of the Verification set to detect non-random classifier performance, based on a one-sided test (power.roc.test, pROC package) for confidence intervals not containing an AUC of 0.5. Results estimated that the blood draw windows containing one-third of the Verification set would have 80% power to detect: AUCs of 0.65 whose 60% confidence intervals did not include 0.5; AUCs of 0.7 whose 80% confidence intervals did not include 0.5; AUCs of 0.75 whose 90% confidence intervals did not include 0.5; and AUCs of 0.8 whose 95% confidence intervals did not include 0.5.

Placenta and Adult Tissue Small RNAseq Data:

Small RNAseq data from the tissue samples were trimmed and mapped using the exceRpt pipeline (Rozowsky et al., Cell Syst 8(4):352-357 (2019)) version 4.6.2 with minimum insert length set at 15 nt and no mismatches permitted. Adult and placenta cell/tissue miRNA data was collected and deconvolution analysis was performed. Scaled data (expressed as reads per million total miRNA reads) (scaled adult and placenta cell/tissue miRNA data were tabulated), scaled expression values averaged for each miRNA, and each cell/tissue type were determined (scaled cell/tissue miRNA data averaged for each cell/tissue type was tabulated)). Differential expression analysis using the sample level data was performed using the Multigroup Comparison function in Qlucore (qlucore.com; Lund, Sweden) was tabulated), and data for highly significantly (q-value <10-12) differentially expressed miRNAs were tabulated (scaled cell/tissue data for miRNAs highly significantly (q-value <10-12) expressed among cell/tissue types).

Estimation of Fractional Contribution of each Cell/Tissue Type to each miRNA

First, deconvolution analysis to calculate the fractional contribution of each cell/tissue type to the overall miRNA content of maternal serum was performed using the CIBERSORT package (Newman et al., Nat Methods 12(5):453-457 (2015)), which employs a linear support vector regression model to estimate proportions. To construct the input dataset, the intersection was taken between the miRNAs that passed the detection filter for the maternal serum extracellular miRNA data and were differentially expressed among tissue types. For the “gene expression signature” input file (gene expression signature data for CIBERSORT analysis, including averaged cell/tissue expression data, was tabulated), the miRNA expression data averaged for each cell/tissue type for the miRNAs in this intersect set was extracted. For the “gene expression profile” input file (gene expression profile data for CIBERSORT analysis, including sample level extracellular miRNA expression data, was tabulated), the raw extracellular miRNA expression data was extracted for each exRNA sample in the Discovery and Verification cohorts for the miRNAs in this intersect set. The CIBERSORT output (CIBERSORT output file, showing for each exRNA sample the percent of the overall miRNA profile accounted for by each cell/tissue type was tabulated) shows for each exRNA sample the percent of the overall miRNA profile accounted for by each cell/tissue type.

The results from the deconvolution analysis were then combined with the miRNA expression values averaged for each cell/tissue type to calculate the contribution of each cell/tissue type to the total expression level for each miRNA. Specifically, for each miRNA and cell/tissue type, the expression of that miRNA in that cell/tissue type was multiplied by the fractional contribution of that cell/tissue type to the overall miRNA profile of maternal serum (averaged across all of the exRNA samples). The resulting values were scaled across cell/tissue types to compute the percent of that miRNA present in maternal serum that was contributed by each cell/tissue type (calculation of the percent contribution of each cell/tissue type to the level of each miRNA in maternal serum was tabulated).

Small RNAseq Data from Immunoprecipitation Experiments:

Small RNAseq data from the tissue samples were trimmed and mapped using the exceRpt pipeline (Rozowsky et al., Cell Syst 8(4):352-357 (2019)) version 4.6.2 with minimum insert length set at 15 nt and no mismatches permitted. Scaled data (expressed as reads per million total miRNA reads) were determined. Multigroup differential expression analysis was performed using Qlucore (Qlucore.com) and miRNAs that were significantly differentially expressed (q<0.05) between at least 2 groups (input, CD63, AGO2, PLAP) were identified.

Results

Maternal serum samples were collected as part of two studies: the Placenta Study at UCSD, and the PAPR Study from Sera Prognostics. The Placenta Study was a single-site high-risk study that enrolled pregnant women with at least one risk factor for placental dysfunction, while the PAPR Study was a multi-site study that enrolled pregnant women without regard to risk factors for placental dysfunction Table 12A. For both studies, subjects were enrolled and maternal serum was collected between 17-28 weeks, and outcomes were obtained after delivery. Nineteen cases and 29 controls were selected from the Placenta Study and 54 cases and 110 controls were selected from the PAPR study (selection criteria are listed in Table 12B). As described above, all of the Placenta Study samples were unblinded and included in the Discovery cohort, while the PAPR Study samples were divided between the Discovery and Verification cohorts in such a way that the Discovery cohort contained 141 subjects and the Verification cohort contained 71 subjects, with similar distributions of cases and controls, and Gestational Age at Blood Draw (GABD) in the two cohorts. There were no significant demographic or clinical differences between the Discovery and Verification cohorts (Table 13). As expected, in both cohorts, there was an earlier median gestational age of delivery and lower mean birthweight in the cases compared to controls. Of the other demographic or clinical variables, only BMI showed a significant difference between cases and controls (Table 13), with the cases having a higher BMI.

TABLE 12A
Enrollment criteria
UCSD PLACENTAL DYSFUNCTION CLINIC
Inclusion Criteria
At least 18 years old and can provide informed consent
Patient is planning a hospital delivery
Singleton gestation
Gestational age between 17w0d and 28w0d inclusive at time of enrollment
Increased risk of placental dysfunction based on one or more of the following:
Abnormal serum analytes
PAPP-A < 0.3 MoM (first trimester)
AFP > 2.5 MoM (second trimester)
hCG > 3.0 MoM (second trimester)
Inhibin > 2.0 MoM (second trimester)
Unconjugated estriol < 0.3 MoM (second trimester)
Previous adverse pregnancy outcome
Severe preeclampsia, eclampsia, or HELLP
Birthweight < 5th percentile
Fetal loss, idiopathic or with known placental dysfunction
Placental abruption
Maternal comorbidity
Chronic hypertension requiring medication
Lupus or other autoimmune disease requiring medication
Chronic renal insufficiency
Exclusion Criteria
Has active or history of malignancy requiring major surgery or systemic chemotherapy
Multiple gestation (including history of twin demise including reduction, spontaneous or
elective)
SERA PROGNOSTICS REPOSITORY
Inclusion Criteria
At least 18 years old and can provide informed consent
Singleton gestation
Gestational age between 17w0d and 28w0d inclusive at time of enrollment
Exclusion Criteria
Multiple gestation
Known or suspected fetal anomaly

TABLE 12B
Selection criteria for cases and controls
UCSD PLACENTAL DYSFUNCTION CLINIC
Case Selection Criteria
New-onset hypertension OR
Chronic hypertension and new-onset proteinuria OR
Chronic proteinuria and new-onset hypertension OR
New-onset or chronic hypertension and new-onset severe feature (elevated liver function tests
(>2x upper limit of the normal range, elevated Creatinine > 1.2, low platelet count <
100,000/uL, and/or intrauterine growth restriction < 10th percentile).
New-onset or chronic proteinuria and new-onset severe feature (elevated liver function tests
(>2x upper limit of the normal range, elevated Creatinine > 1.2, low platelet count < 100,000/uL,
and/or intrauterine growth restriction < 10th percentile).
Control Selection Criteria
No hypertensive disease
SERA PROGNOSTICS REPOSITORY
Case Selection Criteria
New-onset hypertension with our without severe features OR
Superimposed preeclampsia with our without severe features
Control Selection Criteria
No hypertensive disease

TABLE 13
Study subject demographics and clinical characteristics

| | Discovery (n = 141) | | | | | Verification (n = 71) | | | | | |
|---|---|---|---|---|---|---|---|---|---|---|---|
| | Case | | Control | | p- | Case | | Control | | | p- |
| | (n = 49) | | (n = 92) | | value | (n = 24) | | (n = 47) | | p-value | value |
| Mean Maternal | 30.1 ± 6.4 | | 29.0 ± 6.1 | | 0.29 | 26.8 ± 6.7 | | 27.7 ± 6.1 | | 0.59 | 0.88 |
| Age (yr) | | | | | | | | | | | |
| Median | 3 ± 1.7 | | 2 ± 1.7 | | 0.24 | 2 ± 2.5 | | 2 ± 1.0 | | 0.72 | 0.85 |
| Gravidity | | | | | | | | | | | |
| Median Parity | 1 ± 1.1 | | 1 ± 1.5 | | 0.93 | 1 ± 2.3 | | 1 ± 1.2 | | 0.75 | 0.96 |
| Mean BMI | 30.7 ± 10.1 | | 26.1 ± 7.8 | | 0.02 | 33.7 ± 9.6 | | 27.6 ± 7.5 | | 0.01 | 0.95 |
| # Subjects with | 10 | (20.4%) | 9 | (9.8%) | 0.12 | 5 | (20.8%) | 5 | (10.6%) | 0.29 | 1.0 |
| diabetes | | | | | | | | | | | |
| Race/Ethnicity | | | | | | | | | | | 0.11 |
| White-Non | 24 | (49.0%) | 50 | (54.3%) | 0.40 | 7 | (29.2%) | 20 | (42.6%) | 0.57 | |
| Hispanic | | | | | | | | | | | |
| Hispanic | 12 | (24.5%) | 25 | (27.2%) | | 13 | (54.2%) | 17 | (36.2%) | | |
| African- | 7 | (14.3%) | 9 | (9.8%) | | 3 | (12.5%) | 8 | (17.0%) | | |
| American | | | | | | | | | | | |
| Asian | 4 | (8.2%) | 3 | (3.3%) | | 0 | (0.0%) | 1 | (2.1%) | | |
| Pacific Islander | 1 | (2.0%) | 0 | (0.0%) | | 0 | (0.0%) | 0 | (0.0%) | | |
| Other | 1 | (2.0%) | 5 | (5.4%) | | 1 | (4.2%) | 1 | (2.1%) | | |
| Median GABD | 24.3 ± 3.0 | | 24.0 ± 3.1 | | 0.31 | 24.1 ± 2.9 | | 23.5 ± 3.3 | | 0.39 | 0.71 |
| (wk) | | | | | | | | | | | |
| Median GA | 36.3 ± 6.4 | | 39.2 ± 1.2 | | <0.01 | 36.2 ± 3.2 | | 39.3 ± 2.5 | | <0.01 | 0.67 |
| delivery (wk) | | | | | | | | | | | |
| # Preterm | 27 | (55.1%) | 3 | (3.3%) | <0.01 | 15 | (60%) | 3 | (6.5%) | <0.01 | 0.49 |

| | | | | | | | |
|---|---|---|---|---|---|---|---|
| deliveries | | | | | | | |
| Mean | 2589.5 ± 922.7 | 3464.8 ± 465.4 | <0.01 | 2733.6 ± 893.5 | 3213.0 ± 675.9 | 0.03 | 0.87 |
| birthweight (g) | | | | | | | |

| | | | |
|---|---|---|---|
| # IUGR | 11 (7.9%) | 5 (7.0%) | 1.0 |

Given the potential for gestational age (GA)-dependent effects on expression of miRNAs, biomarker discovery and verification were each performed on the entire GA range, as well as for three GA windows: 17 weeks 0 days −21 weeks 5 days (Early); 19 weeks 5 days -24 weeks 4 days (Middle); and 22 weeks 2 days −28 weeks 0 days (Late).

Discovery and Verification of Univariate and Bivariate Predictors

In this study, the aim was to discover and verify individual univariate and bivariate (also termed reversals) predictors with significant AUCs (lower 95% CIs did not include 0.5). In a future verification/validation study, different combinations of multiple verified predictors and clinical parameters are to be tested to identify the best performing test for clinical use.

Data filtering and processing are described in detail above. Briefly, the small RNAseq data were mapped using the exceRpt pipeline (Rozowsky et al., Cell Syst 8(4):352-357 (2019)) and the resulting miRNA data were filtered to remove miRNAs with >70% missing values. Sample-to-sample normalization was carried out through stabilization of variance and reduction in bias across distributions of read counts. Batch normalization was carried out using the PEER package (Astrand, Journal of Computational Biology 10(1):85-102 (2003).

In the Discovery phase of the univariate analysis, individual miRNAs with single test significance (p<0.05) were selected for each GABD window, resulting in identification of 14 individual miRNAs for the entire GABD window, 14 for the Early GABD window, 11 for the Middle GABD window, and 21 for the Late GABD window (Table 14 and Table 15). In the Verification phase of the univariate analysis, miRNAs for which the lower 95% confidence interval (CI) of the area under the curve (AUC) was >0.5 were considered to have passed Verification. This analysis identified two candidate individual miRNAs that passed Verification, one for the Early GA window (hsa-miR- 516b-5p) and one for the Middle GA window (hsa-miR-941) (Tables 14, 15 and 18). This verification rate is not unexpected given the size of the verification set.

TABLE 14
Tally of candidate univariate predictors and reversals selected in discovery
and passing verification

| | Univariate Analysis | | Bivariate Analysis | |
|---|---|---|---|---|
| | #Pass-filter | #Pass-filter | | #Pass-filter Verification |
| GABD window | Discovery, x² | Verification (Lower | #Selected | (Lower 95% CI AUC > |
| (days) | p < 0.05 | 95% CI AUC > 0.5) | Discovery | 0.5) |
| Full (119-202) | 14 | 0 | 50 | 1 |
| Early (119-152) | 14 | 1 | 50 | 4 |
| Middle (138-172) | 11 | 1 | 50 | 2 |
| Late (156-196) | 21 | 0 | 50 | 23 |

Normalization of extracellular miRNA datasets can be challenging. Standard normalization approaches, such as the use of spike-in synthetic oligonucleotides, “housekeeping” small RNAs, or bioinformatic methods commonly used for cellular long RNAseq datasets have not been successful. Even for studies of miRNAs in cells and tissues, it has been advocated that sample set-specific normalizers be used (Schwarzenbach et al., Clin Chem 61(11):1333-1342 (2015)), and it is commonly accepted that normalization of exRNA datasets is even more challenging (Endzelins et al., BMC Cancer 17(1):730 (2017)). It was reasoned that for pairs of endogenous miRNAs, the expression of each miRNA might serve as an endogenous control for the other and therefore produce more reproducible features than the abundances measured for individual miRNAs. This paired normalization approach was implemented by forming ratios of individual miRNA abundances, termed “reversals”, by adapting the method described in Price et al. (Price et al., Proc Natl Acad Sci USA 104(9):3414-3419 (2007)). In pregnancy, this approach has been applied to the development of prognostic biomarkers of spontaneous preterm birth and preeclampsia based on serum protein abundances (Saade et al., Am J Obstet Gynecol 214(5):633 (2016); Verlohren et al., Am J Obstet Gynecol 206(1):58 (2012)). After log transformation, the miRNA abundance data approximated a normal distribution, enabling assessment of relative expression in an arithmetic, geometric or power relationship. While geometric relationships (calculated as log ratios) are commonly used to generate models from biological data and, power relationships (calculated as ratios of normally distributed values) are a feature of risk analysis (Hayya et al., Management Science 21(11):1338-1341 (1975)), and have been used successfully in analysis of cDNA microarrays (Chen et al., J Biomed Opt 2(4):364-374 (1997)). For the Discovery cohort, the results obtained when the reversals were constructed as the log values of the ratios of normalized counts (geometric) and the ratios of the log values (power) were compared. It was found that the latter was preferred because it resulted in better separation of cases and controls, as visualized in the first two principal components of a Principal Component Analysis (PCA) (FIGS. 1A and 1B). The reversals constructed by the ratios of the log values also resulted in increased stability and magnitude of performance in LASSO analysis. Thus, this approach was used for generation of bivariate features (reversals) for both the Discovery and Verification portions of these studies.

Reversals were selected by ranking performance in bootstrapped resampling with replacement, as detailed as described above. Briefly, five ranks were derived from the following statistics, each computed across 1000 iterations of cross-validation: 1) the mean of the cross-validation AUCs; 2) the lower 25th percentile of the cross-validation AUCs; 3) the mean of the squared Pearson correlation coefficient between the reversal scores with diagnosis of Preeclampsia Case (1) or Control (0); 4) the lower 25th percentile of the squared Pearson correlation coefficient between the reversal scores and the diagnosis of Preeclampsia Case (1) or Control (0); and 5) the square of the differences in the case mean and control mean reversal scores (i.e., the squared mean shift). Each rank was then inverted and all ranks were summed for each reversal to obtain the final ranking.

For the bivariate analysis, the top 50 reversals for each GABD window were selected for testing in the Verification phase, in which those for which the lower 95% CI AUC >0.5 were considered to have passed Verification (Table 16). This analysis identified one reversal in the Full GABD window, four in the Early window, two in the Middle window, and 23 in the Late window, that passed Verification in the same window (Tables 14, 16 and 18). The verification rates are not unexpected given the size of the verification set, with the exception of an unusually high rate of verification in the Late window.

Overall, it was observed that the verification rate was markedly higher for the reversals than for the univariate predictors (Table 14), and for the Late GABD window than for the other three GABD windows. The superior performance of reversals compared to univariate predictors was attributed to the “internal normalization” gained by using a ratio of values for a pair of miRNAs measured in the same sample, which would be expected to minimize the technical variability that might be introduced during sample collection, processing, storage, and analysis. The Late GABD window may have the best performance because of the larger number of samples (compared to the Early and Middle GABD windows), and lower GA-dependent biological variability (compared to the Full GABD window). Another indication that bivariate analysis may provide more robust results than univariate analysis was seen when examining the relative expression of the individual miRNAs comprising the reversals in cases compared to controls (Table 17: Discovery (Num/Denom): Direction (case vs control (ctrl))/ Wilcoxon p-value and Verification (Num/Denom): Direction (case vs ctrl)/ Wilcoxon p-value). Here, it was seen that when examined individually, 4/9 of the denominator miRNAs were significantly (Wilcoxon p<0.05, indicated in red font) differentially expressed between cases and controls in the Discovery set while 2/9 were also differentially expressed in the Verification set. However, for the individual component miRNAs in the reversals, the direction of differential expression was not always preserved between the Discovery and Verification cohorts. Taken together, these findings support that the relative concentrations of pairs of extracellular miRNAs, rather than the absolute levels of individual miRNAs, are more robust predictive biomarkers of disease.

A strong correlation was observed (r2=0.309) between the Discovery and Verification AUCs for all verified univariate predictors and reversals, indicating similar performance in two independent sets of subjects.

For the Early, Middle, and Late GABD windows, the normalized reversal scores from all cases and controls were used for the verified reversals to generate PCA plots. Principal component analysis was performed on normalized reversal scores for verified reversals calculated for both Discovery and Verification subjects. All PCA plots were generated using unsupervised clustering of preeclampsia cases and non-case controls. For the Full GABD windows, there was one verified reversal, which was not sufficient to generate a PCA plot. The PCA plots for the Early and Late GABD windows showed good separation between cases and controls. For the Middle GABD window, outlier cases and controls were most clearly separated, which may be due to the small number (two) of verified reversals in this GABD window. To examine the relationships between reversal scores and diagnosis (cases and controls) on a more granular level, heatmaps were generated for the top 50 reversals for each GABD window. As would be expected, clustering of cases and controls and segregation from one and other and intermixing of samples from the Discovery and Verification sets was observed. It was also observed that for each of the verified reversals, there were several other reversals (which often shared either the numerator or denominator miRNA) that displayed similar patterns but did not pass verification. Reversals that passed verification were: (A) Early GABD window, for the Early GABD subjects (Discovery and Verification sets) (miR.155.5p/miR.3173.5p, miR.1273h.3p/miR.3173.5p, miR.4732.3p/miR.381.3p, miR.4732.3p/miR.941); (B) Middle GABD window for the Middle GABD subjects (miR.1285.3p/mir.378c, miR.150.3p/miR.193b.5p); (C) Late GABD window for the Late GABD subjects (let.7b.5p/miR.155.5p, miR.423.5p/miR.155.5p, miR.25.3p/miR.155.5p, miR.30d.5p/miR.155.5p, miR.151a.3p/miR.155.5p, let.7g.5p/miR.155.5p, let.7i.5p/miR.155.5p, miR.451a/miR.155.5p, miR.126.3p/miR.155.5p, miR.26a.5p/miR.155.5p, miR.425.5p/miR.155.5p, miR.181a.5p/miR.155.5p, miR.363.3p/miR.155.5, miR.320a/miR.155.5p, miR.320b/miR.155.5p, miR.99a.5p/miR.155.5p, miR.125a.5p/miR.155.5p, miR.625.3p/miR.155.5p, miR.146b.5p/miR.155.5p, miR.146a.5p/miR.155.5p, miR.4443/miR.155.5p, miR.516b.5p/miR.155.5p, miR.26b.5p/miR.155.5p); (D) Full GABD window for all subjects (miR.127.3p/miR.485.5p). This suggests that there may be certain “high-value” numerators and denominators (especially hsa-miR-485-5p for the Late and Full GABD windows) that can be used as components of potential multivariate predictors. As well, verification of additional reversals can be obtained with a larger data set.

Predictors Discovered in One GABD Window can Verify in other GABD Windows

It is of clinical interest to identify predictors that perform well across a broad range of gestational ages. Thus, in addition to determining whether each univariate predictor and reversal identified in Discovery was verified in the same GABD window, its performance across all four GABD windows was determined. It was found that several predictors passed the Verification threshold (5th percentile AUC >0.5) in other GABD windows (italicized in Table 17, Verification: GABD window). For each predictor that verified in at least one GABD window, the mean AUC for each GABD window for both the Discovery and Verification cohorts was provided (Table 17, Discovery Mean AUC: Full, Early, Middle, Late and Verification Mean AUC: Full, Early, Middle, Late). These results showed that some of the predictors, particularly hsa-miR-127-3p/hsa-miR-485-5p, hsa-miR-1285-3p/hsa-mir-378c, and hsa-miR-331-3p (mean Verification AUCs bolded in Column C (numerator) and D (Denominator) in Table 17), performed well in the Verification cohort across all GABD windows.

Some of the same miRNAs are Shared among Multiple Reversals, or between Univariate Predictors and Reversals

Four miRNAs (hsa-miR-485-5p, hsa-miR-941, hsa-miR-3173-5p, and hsa-miR-155-5p) were found in the denominators of more than one reversal, with hsa-miR-155-5p being in the denominator of all 23 of the reversals discovered and verified in the Late GABD window (Late/Late), as well as in the numerator of one of the Early/Early reversals. hsa-miR-26b-5p was the numerator in one Late/Late and one Full, Late/Middle reversal. Both of the univariate predictors that were discovered and verified in the same GABD window (hsa-miR-516b-5p and hsa-miR-941) were also members of verified reversals (highlighted in red and blue in Table 18).

Verified Predictors Include Placenta-Associated miRNAs

Verified predictors include members of two placenta-associated miRNA clusters, one located on Chromosome 14 (Seitz et al., Genome Res 14:1741-1748 (2004)) (Tables 17 and 18, Numerator chromosome, Denominator chromosome, and In miRNA Cluster) and the other on Chromosome 19 (Bentwich et al., Nature Genetics 37:766 (2005)) (Tables 17 and 18, Numerator chromosome, Denominator chromosome, and In miRNA Cluster).

To assess the likely cell/tissue source of the miRNAs comprising each predictor, small RNAseq was performed on: Peripheral Blood Mononuclear Cells (PBMCs), Red Blood Cells (RBCs) and platelets collected by centrifugation from human plasma; Granulocytes, Lymphocytes, and Monocytes isolated from human plasma by fluorescence activated cell sorting; and adult human Brain, Heart, Intestine, Kidney, Liver, Lung, and Pancreas, and human Placenta collected from 17-28 weeks gestation (Sample level data: Data averaged for each cell/tissue type). miRNAs that are highly significantly (q-value <10-12) differentially expressed among cell/tissue types were identified and combined with the raw exRNA data from the Discovery and Verification cohorts for deconvolution analysis to estimate the fractional contribution of each cell/tissue type to the overall extracellular miRNA profile of maternal serum. Briefly, for cell and tissue miRNA expression and deconvolution analysis, a PCA plot showing unsupervised clustering of cell and tissue types by miRNA profiling data was generated. In addition, hierarchical clustering of cell and tissue types with heatmap of differentially expressed miRNAs (q-value<10-12) was generated. A box-and-whisker plot of deconvolution results was generated, indicating the percent contribution of each cell/tissue type to the extracellular miRNA profiles of each of the maternal serum samples in this study (both Discovery and Verification cohorts). Finally, the results of the deconvolution analysis were combined with the averaged cell/tissue miRNA expression data to estimate the fractional contribution of each cell/tissue type to the amount of each extracellular miRNA in maternal serum. This information was then extracted for the miRNAs comprising each univariate predictor and reversal (Table 17, Tissue Atlas columns), and the cell/tissue types contributing the highest percentage, or a percentage within ten percentage points of the highest percentage, were listed (Table 17: Tissue Atlas, Max/(Max-10%)). It was observed that Liver, RBC, Placenta, and Platelets contributed most strongly to the overall maternal extracellular miRNA population. These cell/tissue types were also the predominant sources of many of the extracellular miRNAs predictors (Table 17: Tissue Atlas, Max/(Max-10%)), but Liver was underrepresented for both the numerators and denominators, and Lymphocytes were overrepresented for the denominators. Overall, the placenta was identified as a major contributor for 12 of the 30 verified reversals and 1 of the 2 verified univariate predictors. Interestingly, in the reversals, the same cell/tissue type was a major contributor to both the numerator and denominator for only three reversals.

Placenta-Associated miRNA Predictors are Associated with CD63+and PLAP+ Carrier Subclasses

Recent work supports the existence of distinct carrier subclasses, each of which is associated with a specific repertoire of molecular cargo, including miRNAs (Murillo et al., Cell 177(2):463-477 (2019); Srinivasan et al., Cell 177(2):446-462 (2019)). To explore whether miRNA predictors were carried by specific carrier subclasses, canonical extracellular vesicles (EVs), placenta-associated EVs, and ribonucleoprotein complexes (RNPs) were enriched from pooled serum from third trimester pregnant women using magnetic beads conjugated to antibodies raised against CD63 (a commonly used EV surface marker), PLAP (a placental EV-associated surface marker (Dragovic et al., Biol Reprod 89(6):151 (2013)), and AGO2 (a component of the RNA-induced silencing complex, and associated with a large fraction of the extracellular miRNAs that are not associated with EVs (Turchinovich et al., Nucleic Acids Res 39(16):7223-7233 (2011)), respectively. Small RNAseq was performed on these immunoaffinity enriched samples, as well as the input pooled serum, and then identified miRNAs that were significantly (q<0.05) differentially expressed among these groups. Hierarchical clustering allowed identification of eight co-expressed sets of miRNAs, each of which had a characteristic pattern of enrichment in one or more carrier subclass. Briefly, for miRNAs associated with different carrier subclasses, a heatmap was generated showing eight sets of co-expressed miRNAs identified by hierarchical clustering. The three expected sets of miRNAs that showed non-overlapping associations with CD63, AGO2, or PLAP indicate that certain miRNAs are loaded into distinct carrier subclasses that display only one of these three markers. The two sets of miRNAs that were enriched for two markers (CD63_AGO2 and CD63_PLAP) suggest that some miRNAs are associated with either two carrier subclasses or with a single carrier subclass displaying both markers. The two sets of miRNAs that were strongly detected in both the Input and associated with one of the markers (Input_CD63 and Input_AGO2) are consistent with certain miRNAs being associated with two carrier subclasses, one displaying either CD63 or AGO2 and one that does not display any of the three tested markers. Finally, the set of miRNAs detected in unfractionated pregnant serum but not associated with any of the three tested markers (Input) indicates that there remains one or more other carrier subclasses that do not display any of the three tested markers. Therefore, each of the detected miRNAs was assigned to one of these Carrier Subclass groups: CD63, AGO2, PLAP, CD63_AGO2, CD63_PLAP, Input_CD63, Input_AGO2, or Input.

The carrier subclasses with which each miRNA predictor was associated were then extracted and listed in Table 18: Associated Carrier Subclasses and Table 17: column “Carrier Subclasses”. Of the 13 miRNAs for which Placenta was a major contributor, 6 were associated with the PLAP subclass, 4 with the CD63 subclass, and 3 with none of the categories. It is noteworthy that the denominators for most of the reversals were not assigned to any of the categories. As noted above, if a given miRNA were associated with an as-yet unidentified carrier subclass, it would be expected to be assigned to the “Input” category. Therefore, the unassigned miRNAs are those that are present at similar levels in all tested subclasses, as well as the input; even representation across carrier subclasses would be a good feature for a broadly useful normalizer, and may be why unassigned miRNAs were preferentially selected as denominators for reversals.

In this study, extracellular miRNA biomarkers were identified and verified for prediction of preeclampsia. An important result was that not only were there many fewer univariate predictors compared to reversals identified in the Discovery cohort, but a markedly lower percentage of the univariate candidates passed the AUC cutoff in the Verification cohort. This finding was attributed to variability in small RNAseq data obtained from exRNA samples arising from both experimental variability during the exRNA isolation process and biological variability from heterogeneity in the representation of the various carrier subclasses among serum samples collected from different individuals (as discussed in (Murillo et al., Cell 177(2):463-477 (2019))). These sources of variability cannot be accounted for using standard normalization methods, thus making measurements of individual miRNAs difficult to compare between samples. In the bivariate analyses described above, ratios of pairs of miRNAs (rather than measurements of single miRNAs, as in the univariate analyses) were tested, allowing the two miRNAs in each pair (also referred to as a reversal) to normalize each other. It was decided to include log transformation into the analysis because gene expression levels have been shown to be lognormally distributed (Bengtsson et al., Genome Res 15(10):1388-1392 (2005)). For lognormally distributed data, linear scale analyses are dominated by high outliers and make detection of down-regulation difficult, making log transformation a logical step to incorporate. However, it was initially unclear whether the log values of the ratios, representing a geometric relationship between miRNA abundances, should be used, or the ratios of the log values, representing a power relationship. Therefore both sets of values were calculated for the Discovery cohort, and it was found that there was better separation of cases and controls using the ratios of the log values, even prior to selection of best miRNA reversals. Ratios of normally distributed values, such as our log-transformed miRNA abundances, are used frequently in risk analysis (Hayya et al., Management Science 21(11):1338-1341 (1975)). Ratios of log values have been shown to be particularly useful for examining a change in the rate of incidence of a clinical event. Relative log survival is an unbiased estimate of the relative hazard (Perneger, Contemp Clin Trials 29(5):762-766 (2008)). Thus, for miRNAs whose abundance is related to PE-free pregnancy “survival” versus incidence of PE, the ratios of logs provide a useful metric. Assessed in the combination of training and verification data, the first principal components of the verified reversals in the early, middle and late blood draw windows show a strong separation between cases and controls while capturing the majority of variance. These observations indicate that the reversals are likely to distinguish cases from controls in similar populations. Intriguingly, many of the Verified reversals contained one miRNA highly expressed in Placenta and one likely non-placental miRNA, and no reversals were comprised of two miRNAs that were both highly expressed in placenta. This indicates that miRNAs expressed by non-target tissues can serve as internal normalizers that enable more robust measurement of target tissue-associated exRNA biomarkers, or that normalization of placental to maternal contributions improves predictions for fetal/maternal dyad disease states like PE.

It was observed that several of the predictors that were identified in a given GABD window from the Discovery cohort performed well across multiple GABD windows in the Verification cohort. This indicates that a clinical predictive assay with good performance across a relatively broad GA range can be developed. The performance and robustness of such a test can be enhanced by constructing a multianalyte assay, which in addition to extracellular miRNA predictors, can incorporate clinical parameters and other molecular biomarkers.

Two clusters of miRNAs have been found to be of particular significance in placental biology. A study by Bentwich et al. identified a placenta-specific cluster of miRNAs on the long arm of Chromosome 19 (chrl9q13) (Bentwich et al., Nature Genetics 37:766 (2005)), which is commonly referred to as the C19MC cluster. A subsequent publication found that this cluster was also highly expressed in pluripotent human embryonic stem cells, but was rapidly downregulated during differentiation (Laurent et al., Stem Cells 26(6):1506-1516 (2008)). miRNAs on the long arm of Chromosome 14 (chr14q32) have also been shown to be highly expressed in the placenta and embryonic stem cells, and to regulate gene expression during development (Seitz et al., Genome Res 14:1741-1748 (2004); Laurent et al., Stem Cells 26(6):1506-1516 (2008)). The verified predictors contained several miRNAs that were encoded in the C19MC and chr14q32 miRNA clusters.

miRNA expression data from a variety of cell and tissue types was used to determine the likely sources of the extracellular miRNA biomarkers. For the reversals, Liver, RBC, Placenta, and Platelets were the most frequent major contributors of the numerator miRNAs, and Lymphocytes were the major contributor for the large majority of denominator miRNAs. miRNA expression data from samples enriched from pooled pregnant serum samples by immunoaffinity separation using magnetic beads conjugated to antibodies raised against CD63, AGO2, and PLAP was also used to determine the carrier subclass association of our extracellular miRNA biomarkers. Nearly half of the miRNA biomarkers for which Placenta was a major contributed were associated with PLAP, and a third were associated with CD63, suggesting that placental EVs and canonical EVs are important carriers of placentally-derived extracellular miRNAs. It is important to note that the approach for estimating the contribution of each cell/tissue type to the level of specific miRNAs in the serum assumes that the intracellular level of each miRNA is reflected in the population of miRNAs released by that cell/tissue into the serum. However, there is evidence that there is selective RNA cargo loading into EVs, RNPs, and other carriers (Wei et al., Nat Commun 8(1):1145 (2017)). Moreover, it was recognized that the dataset does not include all cell and tissue types. To refine these calculations, profiles of the exRNAs released by each cell and tissue type can be obtained.

Among the collected demographic and clinical variables, the expected earlier gestational of delivery and lower birthweight in cases compared to controls was observed. A significantly higher BMI in cases compared to controls was noted, which is consistent with previous literature reporting an elevated risk of preeclampsia in obese gravidas (Roberts et al., Pregnancy Hypertens 1(1):6-16 (2011)).

Previous studies have reported on extracellular miRNAs associated with preeclampsia, with limited overlap in identified miRNAs among studies (Gan et al., Medicine (Baltimore) 96(28):e7515 (2017); Gunel et al., Placenta 52:77-85 (2017);b Hromadnikova et al., PLoS One 12(2):e0171756 (2017); Hromadnikova et al., Mediators Inflamm 2013:186041 (2013); Jairajpuri et al., Gene 627:543-548 (2017); Li et al., Biomed Res Int 2013:970265 (2013); Luque et al., Sci Rep 4:4882 (2014); Martinez-Fierro et al., Arch Gynecol Obstet, 297(2):365-371 (2018); Miura et al., J Obstet Gynaecol Res, 41(10):1526-1532 (2015); Motawi et al., Arch Biochem Biophys 659:13-21 (2018); Salomon et al., J Clin Endocrinol Metab 102(9):3182-3194 (2017); Stubert et al., Hypertens Pregnancy 33(2):215-235 (2014); Timofeeva et al., Placenta 61:61-71 (2018); Ura et al., Taiwan J Obstet Gynecol 53(2):232-234 (2014); Wu et al., Reproduction 143(3):389-397 (2012); Xu et al., Hypertension 63(6):1276-1284 (2014); Yang et al., Clin Chim Acta 412(23-24):2167-2173 (2011); Yang et al., Mol Med Rep, 12(1):527-534 (2015); Yoffe et al., Sci Rep 8(1):3401 (2018)). These studies can be divided into twelve discovery studies, which used large qRT-PCR panels, microarrays, or small RNA sequencing (Gunel et al., Placenta 52:77-85 (2017); Li et al., Biomed Res Int 2013:970265 (2013); Luque et al., Sci Rep 4:4882 (2014); Salomon et al., J Clin Endocrinol Metab 102(9):3182-3194 (2017); Stubert et al., Hypertens Pregnancy 33(2):215-235 (2014); Timofeeva et al., Placenta 61:61-71 (2018); Ura et al., Taiwan J Obstet Gynecol 53(2):232-234 (2014); Wu et al., Reproduction 143(3):389-397 (2012); Xu et al., Hypertension 63(6):1276-1284 (2014); Yang et al., Clin Chim Acta 412(23-24):2167-2173 (2011); Yang et al., Mol Med Rep, 12(1):527-534 (2015); Yoffe et al., Sci Rep 8(1):3401 (2018)), and seven targeted studies, which performed qRT-PCR on small numbers of selected miRNAs (Gan et al., Medicine (Baltimore) 96(28):e7515 (2017); Hromadnikova et al., PLoS One 12(2):e0171756 (2017); Hromadnikova et al., Mediators Inflamm 2013:186041 (2013); Jairajpuri et al., Gene 627:543-548 (2017); Martinez-Fierro et al., Arch Gynecol Obstet, 297(2):365-371 (2018); Miura et al., J Obstet Gynaecol Res, 41(10):1526-1532 (2015); Motawi et al., Arch Biochem Biophys 659:13-21 (2018)), most commonly members of the placenta-specific miRNA cluster located at chrl9q13 (C19MC) (Hromadnikova et al., PLoS One 12(2):e0171756 (2017); Hromadnikova et al., Mediators Inflamm 2013:186041 (2013); Jairajpuri et al., Gene 627:543-548 (2017); Martinez-Fierro et al., Arch Gynecol Obstet, 297(2):365-371 (2018); Miura et al., J Obstet Gynaecol Res, 41(10):1526-1532 (2015)). Eleven C19MC miRNAs were identified in four of the discovery studies (the other eight discovery studies did not identify any C19MC miRNAs) (Timofeeva et al., Placenta 61:61-71 (2018); Ura et al., Taiwan J Obstet Gynecol 53(2):232-234 (2014); Yang et al., Clin Chim Acta 412(23-24):2167-2173 (2011); Yang et al., Mol Med Rep, 12(1):527-534 (2015)), but only three specific miRNAs were shared among at least two studies: hsa-miR-517c-3p and hsa-miR-518e-3p were shared between Yang et al. 2011 (Yang et al., Clin Chim Acta 412(23-24):2167-2173 (2011)) and Yang et al. 2014 (Yang et al., Mol Med Rep, 12(1):527-534 (2015)), and hsa-miR-519a-3p was common to Yang et al. 2011 (Yang et al., Clin Chim Acta 412(23-24):2167-2173 (2011)) and Timofeeva et al. (Timofeeva et al., Placenta 61:61-71 (2018)). Of the nineteen C19MC miRNAs identified as differentially expressed between PE and control in at least one targeted study, five were found by two studies. Jairajpuri et al. used a candidate approach, targeting 84 miRNAs identified in previous placental RNA and exRNA studies to be associated with preeclampsia, but found that only 43 of these were detectable in their exRNA samples (Jairajpuri et al., Gene 627:543-548 (2017)). Of these, nine overlapped with extracellular miRNA biomarkers found in other exRNA studies. All five of the overlapping miRNAs that were higher in PE than control in the Jairajpuri et al. data were also consistently higher in the other studies: hsa-miR-650 (Ura et al., Taiwan J Obstet Gynecol 53(2):232-234 (2014)); hsa-miR-29a (Li et al., Biomed Res Int 2013:970265 (2013); Yang et al., Clin Chim Acta 412(23-24):2167-2173 (2011); Yang et al., Mol Med Rep, 12(1):527-534 (2015)); hsa-miR-210 (Gan et al., Medicine (Baltimore) 96(28):e7515 (2017); Ura et al., Taiwan J Obstet Gynecol 53(2):232-234 (2014); Xu et al., Hypertension 63(6):1276-1284 (2014)); hsa-miR-518b (Hromadnikova et al., PLoS One 12(2):e0171756 (2017); Miura et al., J Obstet Gynaecol Res, 41(10):1526-1532 (2015); Ura et al., Taiwan J Obstet Gynecol 53(2):232-234 (2014)); and hsa-miR-155-5p (Gan et al., Medicine (Baltimore) 96(28):e7515 (2017)). Three of the four overlapping miRNAs that were lower in PE than control in the Jairajpuri et al. data were also consistently lower in the other studies: hsa-miR-144-3p (Li et al., Biomed Res Int 2013: 970265 (2013); Ura et al., Taiwan J Obstet Gynecol 53(2):232-234 (2014); Wu et al., Reproduction 143(3):389-397 (2012)); hsa-miR-19b1 (Xu et al., Hypertension 63(6):1276-1284 (2014)); and hsa-miR-15b-5p (Ura et al., Taiwan J Obstet Gynecol 53(2):232-234 (2014)). The fourth overlapping miRNA that was lower in PE than control for the Jairajpuri et al. (Jairajpuri et al., Gene 627:543-548 (2017)) study was also lower in PE in Xu et al. (Xu et al., Hypertension 63(6):1276-1284 (2014)) but higher in PE in Yang et al. (Yang et al., Mol Med Rep, 12(1):527-534 (2015)). It is notable that the C19MC miRNAs were largely seen in the studies that compared PE cases after diagnosis with gestational age-matched non-PE controls. Of the five studies that used a discovery approach on pre-symptomatic subjects (Luque et al., Sci Rep 4:4882 (2014); Salomon et al., J Clin Endocrinol Metab 102(9):3182-3194 (2017); Ura et al., Taiwan J Obstet Gynecol 53(2):232-234 (2014); Xu et al., Hypertension 63(6):1276-1284 (2014); Yoffe et al., Sci Rep 8(1):3401 (2018)), the sample sizes were quite small (15-35 cases/24-40 controls), and only one C19MC miRNA was identified as a biomarker in one study (Ura et al., Taiwan J Obstet Gynecol 53(2):232-234 (2014)). The results were compared to these previous studies, and it was found that 11 of our miRNA predictors overlapped with biomarkers present at higher levels in the serum or plasma of patients with preeclampsia (or who later developed preeclampsia) compared to controls, and 5 overlapped with biomarkers previously reported to be lower in preeclampsia compared to controls. All overlaps between the miRNAs identified in this study with prior studies are shown in Table 17: Overlap with Literature. Of these overlapping miRNAs, miR-155-5p, which was present in 24 of the verified reversals, is of particular interest. It has been reported to be more highly expressed in placentas from preeclamptic compared to normal pregnancies and to suppress cell invasion in HTR-8/SVneo trophoblast cell line through repression of eNOS expression (Li et al., Mol Med Rep 10(1):550-554 (2014)). It was later reported that in Human Umbilical Vein Endothelial Cells (HUVECs), aspirin could prevent TNFa-induced endothelial dysfunction by repressing downstream hsa-miR-155-5p expression and thereby derepressing eNOS (Kim et al., Free Radic Biol Med 104:185-198 (2017)). Therefore, hsa-miR-155-5p can be a biomarker for prediction and diagnosis of preeclampsia, and can also be a functional mediator of preeclampsia pathogenesis.

The extracellular miRNA biomarkers for preeclampsia can be indicators of placental or maternal tissue stress and/or serve as signaling molecules between the placenta and maternal tissues, or between maternal tissues. Three novel extracellular miRNA biomarkers identified in this study have been previously associated with hypertension. hsa-miR-26b-5p and hsa-miR-7-5p were found to be upregulated in the plasma of non-pregnant patients with hypertension and left ventricular hypertrophy (LVH) compared to normotensive patients or patients with hypertension but no LVH (Kaneto et al., Braz J Med Biol Res 50(12):e6211 (2017)). hsa-miR-181a-5p mimic has been shown to decrease blood pressure in hypertensive mice (Marques et al., Adv Exp Med Biol 888:215-235 (2015)). In the analysis, hsa-miR-26b-5p appears to be predominantly derived from the liver and placenta, hsa-miR-7-5p from the brain, and hsa-miR-181a-5p from the placenta.

The studies described above have applied an unbiased approach to discovery and verification of novel extracellular miRNA biomarkers for prediction of preeclampsia. The rigor of the study design, including adequate numbers of cases and controls for both the Discovery and blinded Verification phases of analysis, has allowed for the development of a novel approach to extracellular miRNA biomarker discovery/verification, which will be generalizable to other diseases. The candidate predictors from this study can be validated on a large independent cohort, as individual biomarkers or as components of multianalyte assays, which can include not only combinations of extracellular miRNA predictors, but also clinical parameters, such as history of severe preeclampsia, kidney disease, chronic hypertension or abnormal analytes during first or second trimester screening. Validated clinical assays for predicting the risk of clinically relevant preeclampsia allows targeting of clinical resources to high-risk cases, while sparing low risk patients unnecessary anxiety they will also enable identification of high-risk cases for clinical studies aimed at personalized administration of aspirin, as well as novel preventative and therapeutic modalities.

TABLE 15
Features of univariate predictors selected in discovery and passing verification

| | | | | | Discov- | Discov- | Verifi- | Verifi- | Discovery AUC | | | Verification AUC | | |
|---|---|---|---|---|---|---|---|---|---|---|---|---|---|---|
| | RNA | GABD | | | ery | ery | cation | cation | | Medi- | | | Medi- | |
| RNA | accession | window | disc.chi | ver.chi | p.val | direction | p.val | direction | Lower | an | Upper | Lower | an | Upper |
| hsa-miR- | MIMAT | Full | 2.84E−04 | 1.70E−01 | 1.59E−03 | Control | 9.76E−01 | Control | 0.56 | 0.66 | 0.76 | 0.35 | 0.50 | 0.66 |
| 30c-5p | 0000244 | | | | | Upper | | Upper | | | | | | |
| hsa-miR- | MIMAT | Full | 7.12E−04 | 3.93E−01 | 2.01E−03 | Control | 5.83E−01 | Case | 0.56 | 0.66 | 0.76 | 0.39 | 0.54 | 0.69 |
| 1301-3p | 0005797 | | | | | Upper | | Upper | | | | | | |
| hsa-miR- | MIMAT | Full | 1.44E−03 | 8.79E−01 | 1.78E−03 | Control | 8.61E−01 | Control | 0.57 | 0.66 | 0.75 | 0.36 | 0.51 | 0.66 |
| 23a-3p | 0000078 | | | | | Upper | | Upper | | | | | | |
| hsa-miR- | MIMAT | Full | 4.74E−03 | 7.38E−01 | 5.33E−03 | Control | 7.95E−01 | Control | 0.55 | 0.64 | 0.74 | 0.37 | 0.52 | 0.67 |
| 6842-3p | 0027587 | | | | | Upper | | Upper | | | | | | |
| hsa-miR- | MIMAT | Full | 1.16E−02 | 1.13E−01 | 1.49E−03 | Control | 2.28E−01 | Control | 0.57 | 0.66 | 0.76 | 0.44 | 0.59 | 0.73 |
| 485-5p | 0002175 | | | | | Upper | | Upper | | | | | | |
| hsa-miR- | MIMAT | Full | 1.17E−02 | 6.20E−01 | 2.16E−02 | Control | 7.31E−01 | Case | 0.52 | 0.62 | 0.72 | 0.39 | 0.53 | 0.66 |
| 361-3p | 0004682 | | | | | Upper | | Upper | | | | | | |
| hsa-miR- | MIMAT | Full | 1.19E−02 | 4.73E−01 | 2.09E−02 | Control | 3.42E−01 | Control | 0.52 | 0.62 | 0.72 | 0.42 | 0.57 | 0.72 |
| 191-5p | 0000440 | | | | | Upper | | Upper | | | | | | |
| hsa-miR- | MIMAT | Full | 1.20E−02 | 3.77E−01 | 2.87E−03 | Control | 6.08E−01 | Case | 0.56 | 0.65 | 0.75 | 0.31 | 0.46 | 0.61 |
| 4446-3p | 0018965 | | | | | Upper | | Upper | | | | | | |
| hsa-miR- | MIMAT | Full | 1.52E−02 | 7.68E−01 | 7.60E−03 | Control | 7.58E−01 | Case | 0.54 | 0.64 | 0.74 | 0.39 | 0.52 | 0.66 |
| 6747-3p | 0027395 | | | | | Upper | | Upper | | | | | | |
| hsa-miR- | MIMAT | Full | 2.32E−02 | 8.77E−01 | 4.81E−02 | Control | 8.05E−01 | Control | 0.51 | 0.60 | 0.70 | 0.38 | 0.52 | 0.66 |
| 409-3p | 0001639 | | | | | Upper | | Upper | | | | | | |
| hsa-miR- | MIMAT | Full | 2.80E−02 | 9.77E−01 | 4.62E−02 | Control | 8.61E−01 | Control | 0.50 | 0.60 | 0.70 | 0.34 | 0.49 | 0.63 |
| 224-5p | 0000281 | | | | | Upper | | Upper | | | | | | |
| hsa-miR- | MIMAT | Full | 3.20E−02 | 9.97E−01 | 6.05E−02 | Case | 7.86E−01 | Case | 0.50 | 0.60 | 0.70 | 0.38 | 0.52 | 0.67 |
| 1224-5p | 0005458 | | | | | Upper | | Upper | | | | | | |
| hsa-miR- | MIMAT | Full | 3.95E−02 | 5.81E−01 | 4.96E−02 | Control | 8.23E−01 | Control | 0.50 | 0.60 | 0.70 | 0.37 | 0.52 | 0.67 |
| 423-3p | 0001340 | | | | | Upper | | Upper | | | | | | |
| hsa-miR- | MIMAT | Full | 4.25E−02 | 3.94E−01 | 4.66E−03 | Control | 4.15E−01 | Control | 0.55 | 0.65 | 0.74 | 0.41 | 0.56 | 0.71 |
| 941 | 0004984 | | | | | Upper | | Upper | | | | | | |
| hsa-miR- | MIMAT | Early | 4.83E−03 | 9.88E−01 | 3.72E−03 | Control | 7.59E−01 | Case | 0.62 | 0.77 | 0.93 | 0.23 | 0.55 | 0.87 |
| 30d-5p | 0000245 | | | | | Upper | | Upper | | | | | | |
| hsa-miR- | MIMAT | Early | 1.22E−02 | 3.18E−01 | 6.35E−02 | Control | 7.08E−01 | Control | 0.51 | 0.68 | 0.85 | 0.26 | 0.56 | 0.86 |
| 1323 | 0005795 | | | | | Upper | | Upper | | | | | | |
| hsa-let-7d- | MIMAT | Early | 1.53E−02 | 9.04E−01 | 2.75E−02 | Control | 9.73E−01 | Case | 0.54 | 0.71 | 0.88 | 0.23 | 0.51 | 0.79 |
| 3p | 0004484 | | | | | Upper | | Upper | | | | | | |
| hsa-miR- | MIMAT | Early | 1.87E−02 | 8.18E−01 | 4.67E−02 | Control | 8.65E−01 | Control | 0.53 | 0.69 | 0.85 | 0.20 | 0.53 | 0.86 |
| 191-5p | 0000440 | | | | | Upper | | Upper | | | | | | |
| hsa-miR- | MIMAT | Early | 2.17E−02 | 7.96E−01 | 8.84E−03 | Control | 9.19E−01 | Case | 0.59 | 0.75 | 0.90 | 0.20 | 0.52 | 0.84 |
| 518e-5p- | 0005450 | | | | | Upper | | Upper | | | | | | |
| hsa-miR- | | | | | | | | | | | | | | |
| 519a-5p- | | | | | | | | | | | | | | |
| hsa-miR- | | | | | | | | | | | | | | |
| 519b-5p- | | | | | | | | | | | | | | |
| hsa-miR- | | | | | | | | | | | | | | |
| 519c-5p- | | | | | | | | | | | | | | |
| hsa-miR- | | | | | | | | | | | | | | |
| 522-5p- | | | | | | | | | | | | | | |
| hsa-miR- | | | | | | | | | | | | | | |
| 523-5p | | | | | | | | | | | | | | |
| hsa-miR- | MIMAT | Early | 2.37E−02 | 5.74E−02 | 9.30E−04 | Control | 7.36E−02 | Control | 0.66 | 0.81 | 0.95 | 0.50 | 0.75 | 1.00 |
| 516b-5p | 0002859 | | | | | Upper | | Upper | | | | | | |
| hsa-miR- | MIMAT | Early | 2.53E−02 | 5.29E−01 | 3.37E−02 | Control | 5.16E−01 | Case | 0.54 | 0.70 | 0.87 | 0.28 | 0.60 | 0.91 |
| 26a-5p | 0000082 | | | | | Upper | | Upper | | | | | | |
| hsa-miR- | MIMAT | Early | 2.77E−02 | 5.79E−01 | 1.11E−01 | Control | 9.19E−01 | Control | 0.45 | 0.66 | 0.86 | 0.20 | 0.48 | 0.76 |
| 99b-5p | 0000689 | | | | | Upper | | Upper | | | | | | |
| hsa-miR- | MIMAT | Early | 3.00E−02 | 2.29E−01 | 1.79E−02 | Control | 2.56E−01 | Control | 0.55 | 0.73 | 0.90 | 0.42 | 0.67 | 0.91 |
| 18a-3p | 0002891 | | | | | Upper | | Upper | | | | | | |
| hsa-miR- | MIMAT | Early | 3.53E−02 | 3.30E−01 | 2.94E−02 | Case | 2.27E−01 | Control | 0.54 | 0.71 | 0.88 | 0.36 | 0.68 | 0.99 |
| 1224-5p | 0005458 | | | | | Upper | | Upper | | | | | | |
| hsa-miR- | MIMAT | Early | 3.66E−02 | 7.49E−01 | 1.00E−01 | Control | 5.62E−01 | Control | 0.48 | 0.66 | 0.84 | 0.32 | 0.59 | 0.86 |
| 142-3p | 0000434 | | | | | Upper | | Upper | | | | | | |
| hsa-miR- | MIMAT | Early | 3.73E−02 | 5.39E−02 | 3.60E−02 | Control | 1.01E−01 | Control | 0.53 | 0.70 | 0.88 | 0.47 | 0.74 | 1.00 |
| 423-3p | 0001340 | | | | | Upper | | Upper | | | | | | |
| hsa-miR- | MIMAT | Early | 4.25E−02 | 3.74E−01 | 3.46E−01 | Control | 3.19E−01 | Control | 0.40 | 0.59 | 0.78 | 0.33 | 0.65 | 0.96 |
| 4429 | 0018944 | | | | | Upper | | Upper | | | | | | |
| hsa-miR- | MIMAT | Early | 4.62E−02 | 6.10E−01 | 7.14E−02 | Control | 5.62E−01 | Case | 0.50 | 0.67 | 0.85 | 0.28 | 0.59 | 0.90 |
| 224-5p | 0000281 | | | | | Upper | | Upper | | | | | | |
| hsa-miR- | MIMAT | Middle | 1.51E−03 | 6.17E−01 | 3.98E−04 | Control | 5.09E−01 | Control | 0.66 | 0.78 | 0.89 | 0.36 | 0.57 | 0.79 |
| 23a-3p | 0000078 | | | | | Upper | | Upper | | | | | | |
| hsa-miR- | MIMAT | Middle | 1.62E−03 | 6.22E−01 | 2.11E−02 | Case | 4.59E−01 | Control | 0.53 | 0.68 | 0.84 | 0.36 | 0.58 | 0.81 |
| 4732-5p | 0019855 | | | | | Upper | | Upper | | | | | | |
| hsa-miR- | MIMAT | Middle | 2.59E−03 | 6.66E−01 | 1.82E−03 | Control | 7.04E−01 | Control | 0.60 | 0.75 | 0.89 | 0.32 | 0.54 | 0.77 |
| 122-5p | 0000421 | | | | | Upper | | Upper | | | | | | |
| hsa-miR- | MIMAT | Middle | 3.92E−03 | 3.15E−01 | 5.02E−03 | Control | 2.52E−01 | Control | 0.59 | 0.72 | 0.86 | 0.41 | 0.63 | 0.84 |
| 191-5p | 0000440 | | | | | Upper | | Upper | | | | | | |
| hsa-miR- | MIMAT | Middle | 7.78E−03 | 7.16E−01 | 1.36E−02 | Control | 7.65E−01 | Control | 0.56 | 0.70 | 0.84 | 0.31 | 0.54 | 0.76 |
| 326 | 0000756 | | | | | Upper | | Upper | | | | | | |
| hsa-miR- | MIMAT | Middle | 9.84E−03 | 1.86E−02 | 1.30E−02 | Control | 3.17E−02 | Control | 0.56 | 0.70 | 0.83 | 0.55 | 0.73 | 0.92 |
| 941 | 0004984 | | | | | Upper | | Upper | | | | | | |
| hsa-miR- | MIMAT | Middle | 9.86E−03 | 1.69E−01 | 1.14E−02 | Control | 1.91E−01 | Control | 0.56 | 0.70 | 0.84 | 0.44 | 0.64 | 0.85 |
| 223-3p | 0000280 | | | | | Upper | | Upper | | | | | | |
| hsa-miR- | MIMAT | Middle | 1.19E−02 | 5.21E−01 | 5.99E−02 | Case | 1.00E+00 | Case | 0.49 | 0.65 | 0.82 | 0.28 | 0.50 | 0.72 |
| 374b-5p | 0004955 | | | | | Upper | | Upper | | | | | | |
| hsa-miR- | MIMAT | Middle | 1.32E−02 | 6.31E−01 | 1.14E−02 | Control | 7.65E−01 | Case | 0.56 | 0.70 | 0.84 | 0.30 | 0.54 | 0.77 |
| 324-3p | 0000762 | | | | | Upper | | Upper | | | | | | |
| hsa-miR- | MIMAT | Middle | 2.01E−02 | 5.59E−01 | 1.03E−01 | Control | 7.65E−01 | Control | 0.47 | 0.63 | 0.80 | 0.30 | 0.54 | 0.77 |
| 30c-5p | 0000244 | | | | | Upper | | Upper | | | | | | |
| hsa-miR- | MIMAT | Middle | 3.73E−02 | 4.34E−01 | 3.17E−02 | Control | 7.34E−01 | Control | 0.52 | 0.67 | 0.82 | 0.31 | 0.54 | 0.77 |
| 148a-3p | 0000243 | | | | | Upper | | Upper | | | | | | |
| hsa-miR- | MIMAT | Late | 7.68E−04 | 3.15E−01 | 7.18E−04 | Control | 1.00E−01 | Control | 0.60 | 0.72 | 0.83 | 0.48 | 0.65 | 0.82 |
| 155-5p | 0000646 | | | | | Upper | | Upper | | | | | | |
| hsa-miR- | MIMAT | Late | 8.44E−04 | 3.76E−01 | 1.27E−03 | Control | 8.48E−01 | Control | 0.59 | 0.70 | 0.82 | 0.29 | 0.48 | 0.67 |
| 30c-5p | 0000244 | | | | | Upper | | Upper | | | | | | |
| hsa-miR- | MIMAT | Late | 4.40E−03 | 7.05E−01 | 2.77E−02 | Control | 7.30E−01 | Control | 0.52 | 0.64 | 0.76 | 0.34 | 0.53 | 0.72 |
| 1301-3p | 0005797 | | | | | Upper | | Upper | | | | | | |
| hsa-miR- | MIMAT | Late | 5.35E−03 | 4.12E−01 | 3.12E−03 | Control | 3.76E−01 | Control | 0.58 | 0.69 | 0.80 | 0.40 | 0.58 | 0.77 |
| 23a-3p | 0000078 | | | | | Upper | | Upper | | | | | | |
| hsa-miR- | MIMAT | Late | 5.67E−03 | 6.58E−01 | 7.19E−03 | Control | 9.29E−01 | Control | 0.55 | 0.67 | 0.79 | 0.33 | 0.51 | 0.69 |
| 10a-5p | 0000253 | | | | | Upper | | Upper | | | | | | |
| hsa-miR- | MIMAT | Late | 7.19E−03 | 8.10E−02 | 3.21E−03 | Control | 1.76E−01 | Control | 0.57 | 0.69 | 0.80 | 0.45 | 0.63 | 0.81 |
| 485-5p | 0002175 | | | | | Upper | | Upper | | | | | | |
| hsa-miR- | MIMAT | Late | 9.47E−03 | 9.81E−01 | 1.74E−02 | Control | 6.17E−01 | Control | 0.53 | 0.65 | 0.77 | 0.36 | 0.55 | 0.74 |
| 4446-3p | 0018965 | | | | | Upper | | Upper | | | | | | |
| hsa-miR- | MI0000783 | Late | 1.48E−02 | 3.49E−01 | 2.71E−02 | Control | 3.23E−01 | Control | 0.52 | 0.64 | 0.76 | 0.42 | 0.59 | 0.77 |
| 375 | | | | | | Upper | | Upper | | | | | | |
| hsa-miR- | MIMAT | Late | 1.65E−02 | 2.76E−01 | 5.11E−02 | Control | 3.62E−01 | Control | 0.51 | 0.62 | 0.74 | 0.41 | 0.59 | 0.77 |
| 6842-3p | 0027587 | | | | | Upper | | Upper | | | | | | |
| hsa-miR- | MIMAT | Late | 2.13E−02 | 5.67E−01 | 2.23E−02 | Control | 8.28E−01 | Control | 0.53 | 0.65 | 0.77 | 0.30 | 0.48 | 0.66 |
| 184 | 0000454 | | | | | Upper | | Upper | | | | | | |
| hsa-miR- | MIMAT | Late | 2.28E−02 | 7.71E−01 | 9.24E−02 | Case | 6.91E−01 | Control | 0.48 | 0.61 | 0.73 | 0.36 | 0.54 | 0.72 |
| 18a-3p | 0002891 | | | | | Upper | | Upper | | | | | | |
| hsa-miR- | MIMAT | Late | 2.92E−02 | 8.02E−01 | 1.15E−02 | Control | 8.08E−01 | Case | 0.54 | 0.66 | 0.78 | 0.34 | 0.52 | 0.70 |
| 6747-3p | 0027395 | | | | | Upper | | Upper | | | | | | |
| hsa-miR- | MIMAT | Late | 3.26E−02 | 7.53E−01 | 7.59E−02 | Control | 9.90E−01 | Case | 0.49 | 0.61 | 0.73 | 0.31 | 0.50 | 0.68 |
| 664a-5p | 0005948 | | | | | Upper | | Upper | | | | | | |
| hsa-miR- | MIMAT | Late | 3.39E−02 | 6.44E−01 | 7.32E−02 | Control | 3.48E−01 | Case | 0.49 | 0.61 | 0.74 | 0.40 | 0.59 | 0.78 |
| 345-5p | 0000772 | | | | | Upper | | Upper | | | | | | |
| hsa-miR- | MIMAT | Late | 3.60E−02 | 6.29E−01 | 6.92E−02 | Case | 9.08E−01 | Control | 0.50 | 0.62 | 0.73 | 0.32 | 0.51 | 0.70 |
| 1260b | 0015041 | | | | | Upper | | Upper | | | | | | |
| hsa-miR- | MIMAT | Late | 3.69E−02 | 9.47E−01 | 1.52E−01 | Case | 8.28E−01 | Case | 0.47 | 0.59 | 0.71 | 0.34 | 0.52 | 0.70 |
| 516b-5p | 0002859 | | | | | Upper | | Upper | | | | | | |
| hsa-miR- | MIMAT | Late | 3.84E−02 | 5.77E−01 | 9.74E−02 | Case | 5.13E−01 | Case | 0.48 | 0.61 | 0.74 | 0.38 | 0.56 | 0.75 |
| 374b-5p | 0004955 | | | | | Upper | | Upper | | | | | | |
| hsa-miR- | MIMAT | Late | 3.98E−02 | 7.98E−01 | 5.11E−02 | Control | 6.73E−01 | Control | 0.51 | 0.62 | 0.74 | 0.35 | 0.54 | 0.73 |
| 1273h-3p | 0030416 | | | | | Upper | | Upper | | | | | | |
| hsa-miR- | MIMAT | Late | 4.25E−02 | 3.62E−01 | 4.36E−02 | Case | 3.10E−01 | Case | 0.51 | 0.63 | 0.75 | 0.42 | 0.60 | 0.77 |
| 99b-3p | 0004678 | | | | | Upper | | Upper | | | | | | |
| hsa-miR- | MIMAT | Late | 4.44E−02 | 3.69E−01 | 6.80E−02 | Control | 3.76E−01 | Control | 0.50 | 0.62 | 0.74 | 0.41 | 0.58 | 0.76 |
| 409-3p | 0001639 | | | | | Upper | | Upper | | | | | | |
| hsa-miR- | MIMAT | Late | 4.66E−02 | 4.95E−02 | 5.63E−02 | Control | 8.03E−02 | Control | 0.50 | 0.62 | 0.74 | 0.49 | 0.66 | 0.84 |
| 331-3p | 0000760 | | | | | Upper | | Upper | | | | | | |

TABLE 16
Reversals selected in discovery and passing verification

| | | | | | Discovery | | | | |
|---|---|---|---|---|---|---|---|---|---|
| Bio- | | | Numerator | Denominator | GABD | Discovery | Verification AUC_Full | | |
| marker(s) | Numerator | Denominator | accession | accession | Window | Rank | Lower | Median | Upper |
| 1 | hsa-miR-7-5p | hsa-miR-485-5p | MIMAT0000252 | MIMAT0002175 | Full | 1 | 0.473 | 0.620 | 0.766 |
| 2 | hsa-miR-501-3p | hsa-miR-4446-3p | MIMAT0004774 | MIMAT0018965 | Full | 2 | 0.396 | 0.543 | 0.689 |
| 3 | hsa-miR-140-3p | hsa-miR-485-5p | MIMAT0004597 | MIMAT0002175 | Full | 3 | 0.465 | 0.606 | 0.748 |
| 4 | hsa-miR-181a-5p | hsa-miR-130b-5p | MIMAT0000256 | MIMAT0004680 | Full | 4 | 0.375 | 0.520 | 0.664 |
| 5 | hsa-miR-484 | hsa-miR-485-5p | MIMAT0002174 | MIMAT0002175 | Full | 5 | 0.391 | 0.537 | 0.683 |
| 6 | hsa-mir-320b-2 | hsa-miR-130b-5p | MI0003839 | MIMAT0004680 | Full | 6 | 0.355 | 0.501 | 0.646 |
| 7 | hsa-miR-501-3p | hsa-miR-485-5p | MIMAT0004774 | MIMAT0002175 | Full | 7 | 0.461 | 0.607 | 0.753 |
| 8 | hsa-miR-100-5p | hsa-miR-485-5p | MIMAT0000098 | MIMAT0002175 | Full | 8 | 0.393 | 0.556 | 0.719 |
| 9 | hsa-miR-27a-3p | hsa-miR-485-5p | MIMAT0000084 | MIMAT0002175 | Full | 9 | 0.420 | 0.566 | 0.712 |
| 10 | hsa-miR-451a | hsa-miR-130b-5p | MIMAT0001631 | MIMAT0004680 | Full | 10 | 0.319 | 0.465 | 0.610 |
| 11 | hsa-miR-7-5p | hsa-miR-4446-3p | MIMAT0000252 | MIMAT0018965 | Full | 11 | 0.301 | 0.454 | 0.607 |
| 12 | hsa-miR-182-5p | hsa-miR-485-5p | MIMAT0000259 | MIMAT0002175 | Full | 12 | 0.482 | 0.623 | 0.765 |
| 13 | hsa-miR-425-5p | hsa-miR-130b-5p | MIMAT0003393 | MIMAT0004680 | Full | 13 | 0.356 | 0.501 | 0.646 |
| 14 | hsa-miR-363-3p | hsa-miR-130b-5p | MIMAT0000707 | MIMAT0004680 | Full | 14 | 0.332 | 0.477 | 0.622 |
| 15 | hsa-miR-140-3p | hsa-miR-4446-3p | MIMAT0004597 | MIMAT0018965 | Full | 15 | 0.373 | 0.522 | 0.672 |
| 16 | hsa-miR-320b | hsa-miR-130b-5p | MIMAT0005792 | MIMAT0004680 | Full | 16 | 0.337 | 0.485 | 0.633 |
| 17 | hsa-let-7b-5p | hsa-miR-130b-5p | MIMAT0000063 | MIMAT0004680 | Full | 17 | 0.381 | 0.527 | 0.674 |
| 18 | hsa-miR-134-5p | hsa-miR-130b-5p | MIMAT0000447 | MIMAT0004680 | Full | 18 | 0.362 | 0.511 | 0.659 |
| 19 | hsa-miR-125a-5p | hsa-miR-130b-5p | MIMAT0000443 | MIMAT0004680 | Full | 19 | 0.464 | 0.608 | 0.753 |
| 20 | hsa-miR-125b-5p | hsa-miR-130b-5p | MIMAT0000423 | MIMAT0004680 | Full | 20 | 0.395 | 0.539 | 0.683 |
| 21 | hsa-miR-182-5p | hsa-miR-4446-3p | MIMAT0000259 | MIMAT0018965 | Full | 21 | 0.378 | 0.528 | 0.679 |
| 22 | hsa-miR-181a-5p | hsa-miR-223-5p | MIMAT0000256 | MIMAT0004570 | Full | 22 | 0.344 | 0.496 | 0.649 |
| 23 | hsa-miR-378g | hsa-miR-485-5p | MIMAT0018937 | MIMAT0002175 | Full | 23 | 0.422 | 0.566 | 0.709 |
| 24 | hsa-let-71-5p | hsa-miR-130b-5p | MIMAT0000415 | MIMAT0004680 | Full | 24 | 0.353 | 0.499 | 0.645 |
| 25 | hsa-miR-127-3p | hsa-miR-485-5p | MIMAT0000446 | MIMAT0002175 | Full | 25 | 0.535 | 0.670 | 0.805 |
| 26 | hsa-miR-363-3p | hsa-miR-485-5p | MIMAT0000707 | MIMAT0002175 | Full | 26 | 0.393 | 0.546 | 0.699 |
| 27 | hsa-miR-140-5p | hsa-miR-379-5p | MIMAT0000431 | MIMAT0000733 | Full | 27 | 0.336 | 0.478 | 0.620 |
| 28 | hsa-miR-125b-5p | hsa-miR-485-5p | MIMAT0000423 | MIMAT0002175 | Full | 28 | 0.357 | 0.511 | 0.664 |
| 29 | hsa-miR-451a | hsa-miR-223-5p | MIMAT0001631 | MIMAT0004570 | Full | 29 | 0.363 | 0.512 | 0.660 |
| 30 | hsa-miR-484 | hsa-miR-4446-3p | MIMAT0002174 | MIMAT0018965 | Full | 30 | 0.372 | 0.520 | 0.669 |
| 31 | hsa-miR-25-3p | hsa-miR-130b-5p | MIMAT0000081 | MIMAT0004680 | Full | 31 | 0.319 | 0.465 | 0.612 |
| 32 | hsa-miR-98-5p | hsa-miR-485-5p | MIMAT0000096 | MIMAT0002175 | Full | 32 | 0.438 | 0.581 | 0.723 |
| 33 | hsa-miR-181a-5p | hsa-miR-485-5p | MIMAT0000256 | MIMAT0002175 | Full | 33 | 0.312 | 0.463 | 0.614 |
| 34 | hsa-miR-199a-3p- | hsa-miR-4446-3p | MIMAT0000232 | MIMAT0018965 | Full | 34 | 0.358 | 0.512 | 0.665 |
| | hsa-miR-199b-3p | | | | | | | | |
| 35 | hsa-miR-181a-5p | hsa-miR-199a-5p | MIMAT0000256 | MIMAT0000231 | Full | 35 | 0.382 | 0.526 | 0.669 |
| 36 | hsa-miR-7-5p | hsa-let-7c-5p | MIMAT0000252 | MIMAT0000064 | Full | 36 | 0.453 | 0.600 | 0.747 |
| 37 | hsa-miR-23b-5p | hsa-miR-2110 | MIMAT0004587 | MIMAT0010133 | Full | 37 | 0.343 | 0.497 | 0.652 |
| 38 | hsa-miR-320a | hsa-miR-130b-5p | MI0000542 | MIMAT0004680 | Full | 38 | 0.333 | 0.481 | 0.630 |
| 39 | hsa-miR-451a | hsa-miR-485-5p | MIMAT0001631 | MIMAT0002175 | Full | 39 | 0.412 | 0.564 | 0.715 |
| 40 | hsa-miR-186-5p | hsa-miR-485-5p | MIMAT0000456 | MIMAT0002175 | Full | 40 | 0.421 | 0.572 | 0.723 |
| 41 | hsa-miR-181a-5p | hsa-miR-941 | MIMAT0000256 | MIMAT0004984 | Full | 41 | 0.361 | 0.505 | 0.649 |
| 42 | hsa-miR-134-5p | hsa-miR-485-5p | MIMAT0000447 | MIMAT0002175 | Full | 42 | 0.290 | 0.434 | 0.577 |
| 43 | hsa-let-7b-5p | hsa-miR-485-5p | MIMAT0000063 | MIMAT0002175 | Full | 43 | 0.432 | 0.576 | 0.720 |
| 44 | hsa-miR-140-5p | hsa-miR-486-3p | MIMAT0000431 | MIMAT0004762 | Full | 44 | 0.386 | 0.529 | 0.672 |
| 45 | hsa-miR-3615 | hsa-miR-130b-5p | MIMAT0017994 | MIMAT0004680 | Full | 45 | 0.325 | 0.476 | 0.627 |
| 46 | hsa-miR-142-5p | hsa-miR-130b-5p | MIMAT0000433 | MIMAT0004680 | Full | 46 | 0.395 | 0.546 | 0.697 |
| 47 | hsa-miR-363-3p | hsa-let-7c-5p | MIMAT0000707 | MIMAT0000064 | Full | 47 | 0.357 | 0.501 | 0.645 |
| 48 | hsa-miR-330-5p | hsa-miR-654-5p | MIMAT0004693 | MIMAT0003330 | Full | 48 | 0.406 | 0.557 | 0.708 |
| 49 | hsa-miR-1307-3p | hsa-miR-130b-5p | MIMAT0005951 | MIMAT0004680 | Full | 49 | 0.334 | 0.483 | 0.632 |
| 50 | hsa-miR-26b-5p | hsa-miR-485-5p | MIMAT0000083 | MIMAT0002175 | Full | 50 | 0.442 | 0.585 | 0.728 |
| 51 | hsa-miR-1224-5p | hsa-miR-433-3p | MIMAT0005458 | MIMAT0001627 | Early | 1 | 0.328 | 0.475 | 0.622 |
| 52 | hsa-miR-125a-3p | hsa-miR-3173-5p | MIMAT0004602 | MIMAT0019214 | Early | 2 | 0.360 | 0.512 | 0.665 |
| 53 | hsa-miR-4732-3p | hsa-miR-381-3p | MIMAT0019856 | MIMAT0000736 | Early | 3 | 0.360 | 0.504 | 0.649 |
| 54 | hsa-miR-4732-3p | hsa-miR-941 | MIMAT0019856 | MIMAT0004984 | Early | 4 | 0.374 | 0.526 | 0.678 |
| 55 | hsa-miR-324-3p | hsa-miR-942-5p | MIMAT0000762 | MIMAT0004985 | Early | 5 | 0.358 | 0.500 | 0.642 |
| 56 | hsa-miR-4433b-3p | hsa-miR-7976 | MIMAT0030414 | MIMAT0031179 | Early | 6 | 0.420 | 0.566 | 0.711 |
| 57 | hsa-miR-370-3p | hsa-miR-193b-5p | MIMAT0000722 | MIMAT0004767 | Early | 7 | 0.479 | 0.613 | 0.748 |
| 58 | hsa-miR-1224-5p | hsa-miR-221-5p | MIMAT0005458 | MIMAT0004568 | Early | 8 | 0.428 | 0.569 | 0.710 |
| 59 | hsa-miR-652-3p | hsa-miR-550a-3-5p | MIMAT0003322 | MIMAT0020925 | Early | 9 | 0.345 | 0.488 | 0.631 |
| 60 | hsa-miR-5189-5p | hsa-miR-374b-5p | MIMAT0021120 | MIMAT0004955 | Early | 10 | 0.455 | 0.598 | 0.740 |
| 61 | hsa-miR-7706 | hsa-miR-193b-5p | MIMAT0030021 | MIMAT0004767 | Early | 11 | 0.444 | 0.584 | 0.725 |
| 62 | hsa-miR-652-3p | hsa-miR-941 | MIMAT0003322 | MIMAT0004984 | Early | 12 | 0.366 | 0.505 | 0.645 |
| 63 | hsa-miR-20a-5p | hsa-miR-3173-5p | MIMAT0000075 | MIMAT0019214 | Early | 13 | 0.397 | 0.545 | 0.693 |
| 64 | hsa-miR-155-5p | hsa-miR-3173-5p | MIMAT0000646 | MIMAT0019214 | Early | 14 | 0.437 | 0.582 | 0.728 |
| 65 | hsa-miR-1292-5p | hsa-miR-221-5p | MIMAT0005943 | MIMAT0004568 | Early | 15 | 0.407 | 0.543 | 0.680 |
| 66 | hsa-miR-19b-3p | hsa-miR-760 | MIMAT0000074 | MIMAT0004957 | Early | 16 | 0.390 | 0.541 | 0.691 |
| 67 | hsa-miR-7-5p | hsa-miR-941 | MIMAT0000252 | MIMAT0004984 | Early | 17 | 0.459 | 0.609 | 0.760 |
| 68 | hsa-miR-330-5p | hsa-miR-942-5p | MIMAT0004693 | MIMAT0004985 | Early | 18 | 0.377 | 0.525 | 0.673 |
| 69 | hsa-miR-1976 | hsa-miR-505-5p | MIMAT0009451 | MIMAT0004776 | Early | 19 | 0.410 | 0.554 | 0.698 |
| 70 | hsa-miR-550a-3-5p- | hsa-miR-193b-5p | MIMAT0020925 | MIMAT0004767 | Early | 20 | 0.586 | 0.711 | 0.836 |
| | hsa-miR-550a-5p | | | | | | | | |
| 71 | hsa-miR-4433b-3p | hsa-miR-378i | MIMAT0030414 | MIMAT0019074 | Early | 21 | 0.544 | 0.676 | 0.807 |
| 72 | hsa-miR-30a-3p | hsa-miR-181b-5p | MIMAT0000088 | MIMAT0000257 | Early | 22 | 0.397 | 0.534 | 0.671 |
| 73 | hsa-miR-150-3p | hsa-miR-3173-5p | MIMAT0004610 | MIMAT0019214 | Early | 23 | 0.402 | 0.554 | 0.706 |
| 74 | hsa-miR-16-2-3p | hsa-miR-941 | MIMAT0004518 | MIMAT0004984 | Early | 24 | 0.352 | 0.509 | 0.666 |
| 75 | hsa-miR-652-3p | hsa-miR-381-3p | MIMAT0003322 | MIMAT0000736 | Early | 25 | 0.332 | 0.476 | 0.620 |
| 76 | hsa-miR-125a-3p | hsa-miR-381-3p | MIMAT0004602 | MIMAT0000736 | Early | 26 | 0.382 | 0.528 | 0.675 |
| 77 | hsa-miR-382-5p | hsa-miR-221-5p | MIMAT0000737 | MIMAT0004568 | Early | 27 | 0.435 | 0.575 | 0.715 |
| 78 | hsa-miR-500a-3p | hsa-miR-146b-3p | MIMAT0002871 | MIMAT0004766 | Early | 28 | 0.432 | 0.574 | 0.715 |
| 79 | hsa-miR-186-5p | hsa-miR-941 | MIMAT0000456 | MIMAT0004984 | Early | 29 | 0.381 | 0.529 | 0.677 |
| 80 | hsa-miR-6741-5p | hsa-miR-221-5p | MIMAT0027383 | MIMAT0004568 | Early | 30 | 0.335 | 0.475 | 0.615 |
| 81 | hsa-miR-106b-5p | hsa-miR-193b-5p | MIMAT0000680 | MIMAT0004767 | Early | 31 | 0.490 | 0.628 | 0.766 |
| 82 | hsa-miR-1249-3p | hsa-miR-204-5p | MIMAT0005901 | MIMAT0000265 | Early | 32 | 0.290 | 0.443 | 0.596 |
| 83 | hsa-miR-2110 | hsa-miR-181b-5p | MIMAT0010133 | MIMAT0000257 | Early | 33 | 0.377 | 0.519 | 0.661 |
| 84 | hsa-miR-144-3p | hsa-miR-942-5p | MIMAT0000436 | MIMAT0004985 | Early | 34 | 0.392 | 0.535 | 0.679 |
| 85 | hsa-miR-885-5p | hsa-miR-146b-3p | MIMAT0004947 | MIMAT0004766 | Early | 35 | 0.426 | 0.561 | 0.697 |
| 86 | hsa-miR-345-5p | hsa-miR-877-5p | MIMAT0000772 | MIMAT0004949 | Early | 36 | 0.322 | 0.470 | 0.618 |
| 87 | hsa-miR-1976 | hsa-miR-378e | MIMAT0009451 | MIMAT0018927 | Early | 37 | 0.351 | 0.499 | 0.647 |
| 88 | hsa-miR-345-5p | hsa-miR-200c-3p | MIMAT0000772 | MIMAT0000617 | Early | 38 | 0.358 | 0.509 | 0.659 |
| 89 | hsa-miR-378e | hsa-miR-221-5p | MIMAT0018927 | MIMAT0004568 | Early | 39 | 0.447 | 0.585 | 0.724 |
| 90 | hsa-miR-485-3p | hsa-miR-381-3p | MIMAT0002176 | MIMAT0000736 | Early | 40 | 0.441 | 0.580 | 0.718 |
| 91 | hsa-miR-3182 | hsa-miR-518e-5p-hsa-miR- | MIMAT0015062 | MIMAT0005450 | Early | 41 | 0.324 | 0.463 | 0.602 |
| | | 519a-5p-hsa-miR-519b-5p- | | | | | | | |
| | | hsa-miR-519c-5p-hsa-miR- | | | | | | | |
| | | 522-5p-hsa-miR-523-5p | | | | | | | |
| 92 | hsa-miR-4433b-3p | hsa-miR-93-3p | MIMAT0030414 | MIMAT0004509 | Early | 42 | 0.457 | 0.601 | 0.746 |
| 93 | hsa-miR-550a-3-5p | hsa-miR-361-5p | MIMAT0020925 | MIMAT0000703 | Early | 43 | 0.403 | 0.549 | 0.695 |
| 94 | hsa-miR-155-5p | hsa-miR-221-5p | MIMAT0000646 | MIMAT0004568 | Early | 44 | 0.406 | 0.541 | 0.675 |
| 95 | hsa-miR-345-5p | hsa-miR-766-5p | MIMAT0000772 | MIMAT0022714 | Early | 45 | 0.356 | 0.497 | 0.638 |
| 96 | hsa-miR-1273h-3p | hsa-miR-3173-5p | MIMAT0030416 | MIMAT0019214 | Early | 46 | 0.474 | 0.620 | 0.766 |
| 97 | hsa-miR-1224-5p | hsa-miR-342-3p | MIMAT0005458 | MIMAT0000753 | Early | 47 | 0.398 | 0.544 | 0.691 |
| 98 | hsa-miR-182-5p | hsa-miR-941 | MIMAT0000259 | MIMAT0004984 | Early | 48 | 0.458 | 0.612 | 0.766 |
| 99 | hsa-miR-320c | hsa-miR-941 | MIMAT0005793 | MIMAT0004984 | Early | 49 | 0.384 | 0.528 | 0.672 |
| 100 | hsa-miR-6852-5p | hsa-miR-505-5p | MIMAT0027604 | MIMAT0004776 | Early | 50 | 0.460 | 0.607 | 0.755 |
| 101 | hsa-miR-877-5p | hsa-miR-24-2-5p | MIMAT0004949 | MIMAT0004497 | Middle | 1 | 0.338 | 0.484 | 0.630 |
| 102 | hsa-miR-92b-3p | hsa-miR-24-2-5p | MIMAT0003218 | MIMAT0004497 | Middle | 2 | 0.376 | 0.525 | 0.674 |
| 103 | hsa-miR-1299 | hsa-miR-433-3p | MIMAT0005887 | MIMAT0001627 | Middle | 3 | 0.405 | 0.551 | 0.698 |
| 104 | hsa-miR-1224-5p | hsa-miR-15b-5p | MIMAT0005458 | MIMAT0000417 | Middle | 4 | 0.393 | 0.533 | 0.673 |
| 105 | hsa-miR-18a-3p | hsa-miR-375 | MIMAT0002891 | MI0000783 | Middle | 5 | 0.419 | 0.560 | 0.701 |
| 106 | hsa-miR-150-3p | hsa-miR-589-5p | MIMAT0004610 | MIMAT0004799 | Middle | 6 | 0.389 | 0.535 | 0.682 |
| 107 | hsa-miR-885-5p | hsa-miR-885-3p | MIMAT0004947 | MIMAT0004948 | Middle | 7 | 0.385 | 0.527 | 0.670 |
| 108 | hsa-miR-206 | hsa-miR-654-3p | MIMAT0000462 | MIMAT0004814 | Middle | 8 | 0.345 | 0.485 | 0.625 |
| 109 | hsa-miR-210-3p | hsa-miR-654-3p | MIMAT0000267 | MIMAT0004814 | Middle | 9 | 0.326 | 0.473 | 0.620 |
| 110 | hsa-miR-532-3p | hsa-miR-374a-5p | MIMAT0004780 | MIMAT0000727 | Middle | 10 | 0.384 | 0.522 | 0.661 |
| 111 | hsa-miR-18a-3p | hsa-miR-942-5p | MIMAT0002891 | MIMAT0004985 | Middle | 11 | 0.447 | 0.597 | 0.746 |
| 112 | hsa-miR-206 | hsa-miR-24-2-5p | MIMAT0000462 | MIMAT0004497 | Middle | 12 | 0.401 | 0.552 | 0.703 |
| 113 | hsa-miR-340-3p | hsa-miR-221-5p | MIMAT0000750 | MIMAT0004568 | Middle | 13 | 0.450 | 0.582 | 0.714 |
| 114 | hsa-miR-181a-2-3p | hsa-miR-374a-5p | MIMAT0004558 | MIMAT0000727 | Middle | 14 | 0.355 | 0.493 | 0.631 |
| 115 | hsa-miR-92b-3p | hsa-miR-589-5p | MIMAT0003218 | MIMAT0004799 | Middle | 15 | 0.326 | 0.471 | 0.615 |
| 116 | hsa-mir-320a | hsa-miR-543 | MI0000542 | MIMAT0004954 | Middle | 16 | 0.356 | 0.499 | 0.642 |
| 117 | hsa-miR-30a-3p | hsa-miR-654-3p | MIMAT0000088 | MIMAT0004814 | Middle | 17 | 0.308 | 0.447 | 0.585 |
| 118 | hsa-miR-4732-5p | hsa-miR-485-5p | MIMAT0019855 | MIMAT0002175 | Middle | 18 | 0.364 | 0.513 | 0.662 |
| 119 | hsa-miR-1285-3p | hsa-mir-378c | MIMAT0005876 | MIMAT0016847 | Middle | 19 | 0.518 | 0.652 | 0.787 |
| 120 | hsa-miR-150-3p | hsa-miR-193b-5p | MIMAT0004610 | MIMAT0004767 | Middle | 20 | 0.572 | 0.700 | 0.829 |
| 121 | hsa-miR-125b-5p | hsa-miR-543 | MIMAT0000423 | MIMAT0004954 | Middle | 21 | 0.367 | 0.516 | 0.665 |
| 122 | hsa-miR-885-5p | hsa-miR-375 | MIMAT0004947 | MI0000783 | Middle | 22 | 0.384 | 0.524 | 0.664 |
| 123 | hsa-miR-1285-3p | hsa-miR-326 | MIMAT0005876 | MIMAT0000756 | Middle | 23 | 0.308 | 0.457 | 0.606 |
| 124 | hsa-mir-320a | hsa-miR-24-2-5p | MI0000542 | MIMAT0004497 | Middle | 24 | 0.333 | 0.480 | 0.628 |
| 125 | hsa-mir-320a | hsa-miR-654-3p | MI0000542 | MIMAT0004814 | Middle | 25 | 0.419 | 0.557 | 0.694 |
| 126 | hsa-miR-20b-5p | hsa-miR-6741-5p | MIMAT0001413 | MIMAT0027383 | Middle | 26 | 0.391 | 0.539 | 0.687 |
| 127 | hsa-miR-4732-5p | hsa-miR-199a-5p | MIMAT0019855 | MIMAT0000231 | Middle | 27 | 0.386 | 0.536 | 0.687 |
| 128 | hsa-miR-877-5p | hsa-miR-589-5p | MIMAT0004949 | MIMAT0004799 | Middle | 28 | 0.397 | 0.540 | 0.682 |
| 129 | hsa-miR-25-5p | hsa-miR-24-2-5p | MIMAT0004498 | MIMAT0004497 | Middle | 29 | 0.332 | 0.484 | 0.636 |
| 130 | hsa-miR-150-3p | hsa-miR-518e-5p-hsa-miR- | MIMAT0004610 | MIMAT0005450 | Middle | 30 | 0.447 | 0.594 | 0.741 |
| | | 519a-5p-hsa-miR-519b-5p- | | | | | | | |
| | | hsa-miR-519c-5p-hsa-miR- | | | | | | | |
| | | 522-5p-hsa-miR-523-5p | | | | | | | |
| 131 | hsa-miR-142-5p | hsa-miR-24-2-5p | MIMAT0000433 | MIMAT0004497 | Middle | 31 | 0.367 | 0.520 | 0.673 |
| 132 | hsa-miR-4746-5p | hsa-miR-326 | MIMAT0019880 | MIMAT0000756 | Middle | 32 | 0.363 | 0.506 | 0.650 |
| 133 | hsa-miR-4732-5p | hsa-miR-4446-3p | MIMAT0019855 | MIMAT0018965 | Middle | 33 | 0.381 | 0.527 | 0.672 |
| 134 | hsa-miR-1285-3p | hsa-miR-6741-5p | MIMAT0005876 | MIMAT0027383 | Middle | 34 | 0.409 | 0.559 | 0.708 |
| 135 | hsa-miR-181b-5p | hsa-miR-24-2-5p | MIMAT0000257 | MIMAT0004497 | Middle | 35 | 0.341 | 0.488 | 0.636 |
| 136 | hsa-miR-1285-3p | hsa-miR-3614-5p | MIMAT0005876 | MIMAT0017992 | Middle | 36 | 0.369 | 0.528 | 0.687 |
| 137 | hsa-miR-4732-5p | hsa-miR-24-2-5p | MIMAT0019855 | MIMAT0004497 | Middle | 37 | 0.390 | 0.544 | 0.698 |
| 138 | hsa-miR-1306-3p | hsa-miR-326 | MIMAT0005950 | MIMAT0000756 | Middle | 38 | 0.455 | 0.595 | 0.735 |
| 139 | hsa-miR-517a-3p- | hsa-miR-375 | MIMAT0002852 | MI0000783 | Middle | 39 | 0.392 | 0.532 | 0.672 |
| | hsa-miR-517b-3p | | | | | | | | |
| 140 | hsa-miR-221-3p | hsa-miR-24-2-5p | MIMAT0000278 | MIMAT0004497 | Middle | 40 | 0.376 | 0.529 | 0.683 |
| 141 | hsa-miR-374b-5p | hsa-miR-342-3p | MIMAT0004955 | MIMAT0000753 | Middle | 41 | 0.369 | 0.517 | 0.664 |
| 142 | hsa-miR-125a-3p | hsa-miR-589-5p | MIMAT0004602 | MIMAT0004799 | Middle | 42 | 0.364 | 0.513 | 0.663 |
| 143 | hsa-miR-4732-5p | hsa-miR-516b-5p | MIMAT0019855 | MIMAT0002859 | Middle | 43 | 0.389 | 0.542 | 0.694 |
| 144 | hsa-miR-4732-5p | hsa-miR-23a-3p | MIMAT0019855 | MIMAT0000078 | Middle | 44 | 0.365 | 0.516 | 0.667 |
| 145 | hsa-miR-374b-5p | hsa-miR-106b-5p | MIMAT0004955 | MIMAT0000680 | Middle | 45 | 0.394 | 0.543 | 0.693 |
| 146 | hsa-miR-4732-5p | hsa-miR-1301-3p | MIMAT0019855 | MIMAT0005797 | Middle | 46 | 0.439 | 0.585 | 0.731 |
| 147 | hsa-miR-1246 | hsa-miR-24-2-5p | MIMAT0005898 | MIMAT0004497 | Middle | 47 | 0.354 | 0.503 | 0.651 |
| 148 | hsa-miR-18a-3p | hsa-miR-19b-3p | MIMAT0002891 | MIMAT0000074 | Middle | 48 | 0.444 | 0.584 | 0.724 |
| 149 | hsa-miR-92b-5p | hsa-miR-654-3p | MIMAT0004792 | MIMAT0004814 | Middle | 49 | 0.363 | 0.505 | 0.648 |
| 150 | hsa-miR-628-3p | hsa-miR-375 | MIMAT0003297 | MI0000783 | Middle | 50 | 0.391 | 0.542 | 0.692 |
| 151 | hsa-miR-378g | hsa-miR-3182 | MIMAT0018937 | MIMAT0015062 | Late | 1 | 0.407 | 0.556 | 0.705 |
| 152 | hsa-mir-320a | hsa-miR-130b-5p | MI0000542 | MIMAT0004680 | Late | 2 | 0.352 | 0.496 | 0.641 |
| 153 | hsa-miR-486-5p | hsa-miR-155-5p | MIMAT0002177 | MIMAT0000646 | Late | 3 | 0.366 | 0.510 | 0.653 |
| 154 | hsa-miR-451a | hsa-miR-155-5p | MIMAT0001631 | MIMAT0000646 | Late | 4 | 0.400 | 0.542 | 0.683 |
| 155 | hsa-miR-125a-5p | hsa-miR-155-5p | MIMAT0000443 | MIMAT0000646 | Late | 5 | 0.369 | 0.512 | 0.656 |
| 156 | hsa-let-7i-5p | hsa-miR-155-5p | MIMAT0000415 | MIMAT0000646 | Late | 6 | 0.412 | 0.552 | 0.693 |
| 157 | hsa-mir-320b-2 | hsa-miR-130b-5p | MI0003839 | MIMAT0004680 | Late | 7 | 0.355 | 0.501 | 0.646 |
| 158 | hsa-let-7b-5p | hsa-miR-155-5p | MIMAT0000063 | MIMAT0000646 | Late | 8 | 0.398 | 0.540 | 0.682 |
| 159 | hsa-miR-25-3p | hsa-miR-155-5p | MIMAT0000081 | MIMAT0000646 | Late | 9 | 0.384 | 0.525 | 0.666 |
| 160 | hsa-miR-516b-5p | hsa-miR-155-5p | MIMAT0002859 | MIMAT0000646 | Late | 10 | 0.393 | 0.537 | 0.682 |
| 161 | hsa-miR-30d-5p | hsa-miR-155-5p | MIMAT0000245 | MIMAT0000646 | Late | 11 | 0.398 | 0.537 | 0.677 |
| 162 | hsa-miR-345-5p | hsa-miR-324-3p | MIMAT0000772 | MIMAT0000762 | Late | 12 | 0.370 | 0.515 | 0.661 |
| 163 | hsa-miR-330-5p | hsa-miR-92b-5p | MIMAT0004693 | MIMAT0004792 | Late | 13 | 0.423 | 0.575 | 0.727 |
| 164 | hsa-miR-320a | hsa-miR-155-5p | MI0000542 | MIMAT0000646 | Late | 14 | 0.399 | 0.541 | 0.683 |
| 165 | hsa-let-7g-5p | hsa-miR-155-5p | MIMAT0000414 | MIMAT0000646 | Late | 15 | 0.387 | 0.529 | 0.672 |
| 166 | hsa-miR-3615 | hsa-miR-155-5p | MIMAT0017994 | MIMAT0000646 | Late | 16 | 0.377 | 0.520 | 0.664 |
| 167 | hsa-miR-98-5p | hsa-miR-485-5p | MIMAT0000096 | MIMAT0002175 | Late | 17 | 0.438 | 0.581 | 0.723 |
| 168 | hsa-miR-151a-3p | hsa-miR-155-5p | MIMAT0000757 | MIMAT0000646 | Late | 18 | 0.397 | 0.535 | 0.674 |
| 169 | hsa-miR-221-3p | hsa-miR-155-5p | MIMAT0000278 | MIMAT0000646 | Late | 19 | 0.393 | 0.531 | 0.669 |
| 170 | hsa-miR-127-3p | hsa-miR-485-5p | MIMAT0000446 | MIMAT0002175 | Late | 20 | 0.535 | 0.670 | 0.805 |
| 171 | hsa-let-71-5p | hsa-miR-485-5p | MIMAT0000415 | MIMAT0002175 | Late | 21 | 0.446 | 0.590 | 0.733 |
| 172 | hsa-miR-423-5p | hsa-miR-155-5p | MIMAT0004748 | MIMAT0000646 | Late | 22 | 0.413 | 0.553 | 0.693 |
| 173 | hsa-miR-1260b | hsa-miR-885-3p | MIMAT0015041 | MIMAT0004948 | Late | 23 | 0.398 | 0.541 | 0.684 |
| 174 | hsa-miR-625-3p | hsa-miR-155-5p | MIMAT0004808 | MIMAT0000646 | Late | 24 | 0.395 | 0.536 | 0.678 |
| 175 | hsa-miR-370-3p | hsa-miR-485-5p | MIMAT0000722 | MIMAT0002175 | Late | 25 | 0.431 | 0.574 | 0.718 |
| 176 | hsa-miR-99a-5p | hsa-miR-155-5p | MIMAT0000097 | MIMAT0000646 | Late | 26 | 0.414 | 0.559 | 0.703 |
| 177 | hsa-miR-20a-5p | hsa-miR-485-5p | MIMAT0000075 | MIMAT0002175 | Late | 27 | 0.440 | 0.585 | 0.730 |
| 178 | hsa-miR-146a-5p | hsa-miR-155-5p | MIMAT0000449 | MIMAT0000646 | Late | 28 | 0.432 | 0.570 | 0.708 |
| 179 | hsa-miR-26a-5p | hsa-miR-155-5p | MIMAT0000082 | MIMAT0000646 | Late | 29 | 0.398 | 0.536 | 0.675 |
| 180 | hsa-miR-134-5p | hsa-miR-485-5p | MIMAT0000447 | MIMAT0002175 | Late | 30 | 0.290 | 0.434 | 0.577 |
| 181 | hsa-miR-181a-5p | hsa-miR-155-5p | MIMAT0000256 | MIMAT0000646 | Late | 31 | 0.379 | 0.521 | 0.664 |
| 182 | hsa-miR-26b-5p | hsa-miR-155-5p | MIMAT0000083 | MIMAT0000646 | Late | 32 | 0.410 | 0.548 | 0.686 |
| 183 | hsa-miR-146b-5p | hsa-miR-155-5p | MIMAT0002809 | MIMAT0000646 | Late | 33 | 0.420 | 0.558 | 0.695 |
| 184 | hsa-miR-320b | hsa-miR-130b-5p | MIMAT0005792 | MIMAT0004680 | Late | 34 | 0.337 | 0.485 | 0.633 |
| 185 | hsa-miR-4443 | hsa-miR-130b-5p | MIMAT0018961 | MIMAT0004680 | Late | 35 | 0.402 | 0.551 | 0.700 |
| 186 | hsa-miR-181a-5p | hsa-miR-130b-5p | MIMAT0000256 | MIMAT0004680 | Late | 36 | 0.375 | 0.520 | 0.664 |
| 187 | hsa-miR-1323 | hsa-miR-485-5p | MIMAT0005795 | MIMAT0002175 | Late | 37 | 0.350 | 0.493 | 0.636 |
| 188 | hsa-miR-126-3p | hsa-miR-155-5p | MIMAT0000445 | MIMAT0000646 | Late | 38 | 0.408 | 0.551 | 0.693 |
| 189 | hsa-miR-26b-5p | hsa-miR-485-5p | MIMAT0000083 | MIMAT0002175 | Late | 39 | 0.442 | 0.585 | 0.728 |
| 190 | hsa-miR-320b | hsa-miR-155-5p | MIMAT0005792 | MIMAT0000646 | Late | 40 | 0.397 | 0.542 | 0.686 |
| 191 | hsa-miR-181a-5p | hsa-miR-485-5p | MIMAT0000256 | MIMAT0002175 | Late | 41 | 0.312 | 0.463 | 0.614 |
| 192 | hsa-miR-425-5p | hsa-miR-155-5p | MIMAT0003393 | MIMAT0000646 | Late | 42 | 0.405 | 0.543 | 0.682 |
| 193 | hsa-let-7b-5p | hsa-miR-485-5p | MIMAT0000063 | MIMAT0002175 | Late | 43 | 0.432 | 0.576 | 0.720 |
| 194 | hsa-miR-320a | hsa-miR-485-5p | MI0000542 | MIMAT0002175 | Late | 44 | 0.433 | 0.577 | 0.721 |
| 195 | hsa-miR-451a | hsa-miR-485-5p | MIMAT0001631 | MIMAT0002175 | Late | 45 | 0.412 | 0.564 | 0.715 |
| 196 | hsa-mir-320a | hsa-miR-485-5p | MI0000542 | MIMAT0002175 | Late | 46 | 0.341 | 0.483 | 0.625 |
| 197 | hsa-miR-185-5p | hsa-miR-485-5p | MIMAT0000455 | MIMAT0002175 | Late | 47 | 0.414 | 0.560 | 0.707 |
| 198 | hsa-miR-363-3p | hsa-miR-155-5p | MIMAT0000707 | MIMAT0000646 | Late | 48 | 0.384 | 0.526 | 0.668 |
| 199 | hsa-miR-4443 | hsa-miR-155-5p | MIMAT0018961 | MIMAT0000646 | Late | 49 | 0.383 | 0.529 | 0.675 |
| 200 | hsa-miR-27a-3p | hsa-miR-485-5p | MIMAT0000084 | MIMAT0002175 | Late | 50 | 0.420 | 0.566 | 0.712 |
| Bio- | Verification AUC_Early | | | Verification AUC_Middle | | | Verification AUC_Late | | |
| marker(s) | Lower | Median | Upper | Lower | Median | Upper | Lower | Median | Upper |
| 1 | 0.184 | 0.529 | 0.875 | 0.499 | 0.693 | 0.887 | 0.492 | 0.666 | 0.839 |
| 2 | 0.304 | 0.559 | 0.813 | 0.410 | 0.623 | 0.836 | 0.394 | 0.584 | 0.775 |
| 3 | 0.225 | 0.529 | 0.834 | 0.461 | 0.667 | 0.872 | 0.493 | 0.663 | 0.834 |
| 4 | 0.309 | 0.618 | 0.926 | 0.261 | 0.491 | 0.721 | 0.324 | 0.510 | 0.695 |
| 5 | 0.284 | 0.618 | 0.951 | 0.440 | 0.649 | 0.858 | 0.423 | 0.601 | 0.779 |
| 6 | 0.372 | 0.676 | 0.981 | 0.243 | 0.465 | 0.687 | 0.381 | 0.563 | 0.744 |
| 7 | 0.207 | 0.510 | 0.813 | 0.456 | 0.662 | 0.868 | 0.449 | 0.630 | 0.811 |
| 8 | 0.302 | 0.637 | 0.972 | 0.181 | 0.412 | 0.644 | 0.393 | 0.591 | 0.790 |
| 9 | 0.148 | 0.520 | 0.891 | 0.328 | 0.557 | 0.786 | 0.457 | 0.635 | 0.812 |
| 10 | 0.318 | 0.627 | 0.937 | 0.286 | 0.518 | 0.749 | 0.281 | 0.469 | 0.656 |
| 11 | 0.325 | 0.627 | 0.930 | 0.371 | 0.592 | 0.813 | 0.418 | 0.615 | 0.813 |
| 12 | 0.125 | 0.461 | 0.796 | 0.532 | 0.728 | 0.925 | 0.546 | 0.712 | 0.877 |
| 13 | 0.314 | 0.618 | 0.922 | 0.275 | 0.504 | 0.734 | 0.340 | 0.526 | 0.713 |
| 14 | 0.322 | 0.637 | 0.952 | 0.272 | 0.509 | 0.745 | 0.303 | 0.488 | 0.673 |
| 15 | 0.293 | 0.588 | 0.883 | 0.376 | 0.601 | 0.826 | 0.407 | 0.594 | 0.780 |
| 16 | 0.441 | 0.735 | 1.000 | 0.328 | 0.561 | 0.795 | 0.355 | 0.541 | 0.727 |
| 17 | 0.359 | 0.657 | 0.954 | 0.268 | 0.513 | 0.758 | 0.304 | 0.493 | 0.681 |
| 18 | 0.243 | 0.559 | 0.874 | 0.308 | 0.544 | 0.779 | 0.299 | 0.495 | 0.691 |
| 19 | 0.397 | 0.696 | 0.995 | 0.322 | 0.557 | 0.793 | 0.418 | 0.606 | 0.793 |
| 20 | 0.394 | 0.667 | 0.940 | 0.291 | 0.522 | 0.753 | 0.306 | 0.495 | 0.684 |
| 21 | 0.331 | 0.637 | 0.943 | 0.397 | 0.618 | 0.840 | 0.437 | 0.623 | 0.809 |
| 22 | 0.306 | 0.657 | 1.000 | 0.213 | 0.443 | 0.673 | 0.413 | 0.594 | 0.775 |
| 23 | 0.338 | 0.637 | 0.937 | 0.429 | 0.636 | 0.843 | 0.333 | 0.519 | 0.706 |
| 24 | 0.264 | 0.588 | 0.912 | 0.247 | 0.487 | 0.727 | 0.326 | 0.512 | 0.698 |
| 25 | 0.466 | 0.696 | 0.927 | 0.479 | 0.675 | 0.872 | 0.497 | 0.675 | 0.854 |
| 26 | 0.298 | 0.637 | 0.976 | 0.350 | 0.575 | 0.799 | 0.440 | 0.625 | 0.810 |
| 27 | 0.266 | 0.510 | 0.754 | 0.320 | 0.539 | 0.759 | 0.348 | 0.534 | 0.719 |
| 28 | 0.317 | 0.696 | 1.000 | 0.358 | 0.579 | 0.800 | 0.228 | 0.411 | 0.594 |
| 29 | 0.299 | 0.647 | 0.995 | 0.305 | 0.539 | 0.774 | 0.394 | 0.575 | 0.755 |
| 30 | 0.334 | 0.637 | 0.940 | 0.384 | 0.610 | 0.835 | 0.384 | 0.567 | 0.751 |
| 31 | 0.309 | 0.618 | 0.926 | 0.253 | 0.496 | 0.738 | 0.293 | 0.481 | 0.669 |
| 32 | 0.334 | 0.637 | 0.940 | 0.542 | 0.728 | 0.915 | 0.433 | 0.606 | 0.779 |
| 33 | 0.206 | 0.569 | 0.932 | 0.362 | 0.588 | 0.813 | 0.419 | 0.601 | 0.783 |
| 34 | 0.228 | 0.569 | 0.909 | 0.388 | 0.610 | 0.831 | 0.268 | 0.457 | 0.645 |
| 35 | 0.130 | 0.422 | 0.713 | 0.296 | 0.518 | 0.739 | 0.301 | 0.486 | 0.671 |
| 36 | 0.182 | 0.539 | 0.896 | 0.201 | 0.439 | 0.676 | 0.383 | 0.567 | 0.752 |
| 37 | 0.133 | 0.451 | 0.769 | 0.307 | 0.548 | 0.789 | 0.291 | 0.490 | 0.690 |
| 38 | 0.361 | 0.657 | 0.953 | 0.309 | 0.553 | 0.796 | 0.333 | 0.524 | 0.716 |
| 39 | 0.218 | 0.578 | 0.939 | 0.408 | 0.623 | 0.838 | 0.446 | 0.627 | 0.809 |
| 40 | 0.207 | 0.559 | 0.911 | 0.373 | 0.601 | 0.829 | 0.440 | 0.623 | 0.805 |
| 41 | 0.319 | 0.627 | 0.936 | 0.383 | 0.596 | 0.810 | 0.426 | 0.601 | 0.776 |
| 42 | 0.158 | 0.451 | 0.744 | 0.203 | 0.417 | 0.631 | 0.372 | 0.558 | 0.743 |
| 43 | 0.223 | 0.569 | 0.914 | 0.477 | 0.671 | 0.865 | 0.459 | 0.635 | 0.810 |
| 44 | 0.168 | 0.490 | 0.812 | 0.410 | 0.623 | 0.835 | 0.382 | 0.563 | 0.743 |
| 45 | 0.402 | 0.696 | 0.990 | 0.310 | 0.557 | 0.804 | 0.336 | 0.526 | 0.717 |
| 46 | 0.289 | 0.559 | 0.829 | 0.240 | 0.478 | 0.716 | 0.373 | 0.570 | 0.766 |
| 47 | 0.309 | 0.637 | 0.965 | 0.267 | 0.496 | 0.725 | 0.326 | 0.507 | 0.688 |
| 48 | 0.222 | 0.569 | 0.915 | 0.292 | 0.522 | 0.751 | 0.397 | 0.582 | 0.766 |
| 49 | 0.429 | 0.725 | 1.000 | 0.290 | 0.535 | 0.780 | 0.352 | 0.538 | 0.724 |
| 50 | 0.282 | 0.598 | 0.914 | 0.521 | 0.706 | 0.891 | 0.470 | 0.647 | 0.823 |
| 51 | 0.442 | 0.706 | 0.969 | 0.416 | 0.632 | 0.847 | 0.397 | 0.582 | 0.767 |
| 52 | 0.500 | 0.716 | 0.932 | 0.385 | 0.601 | 0.816 | 0.338 | 0.531 | 0.725 |
| 53 | 0.619 | 0.804 | 0.989 | 0.235 | 0.456 | 0.677 | 0.379 | 0.560 | 0.741 |
| 54 | 0.622 | 0.804 | 0.986 | 0.398 | 0.632 | 0.865 | 0.506 | 0.680 | 0.854 |
| 55 | 0.386 | 0.627 | 0.869 | 0.253 | 0.469 | 0.686 | 0.340 | 0.522 | 0.703 |
| 56 | 0.324 | 0.588 | 0.853 | 0.324 | 0.539 | 0.755 | 0.313 | 0.502 | 0.692 |
| 57 | 0.309 | 0.598 | 0.887 | 0.480 | 0.675 | 0.871 | 0.442 | 0.618 | 0.793 |
| 58 | 0.479 | 0.745 | 1.000 | 0.443 | 0.649 | 0.855 | 0.374 | 0.558 | 0.741 |
| 59 | 0.248 | 0.549 | 0.850 | 0.293 | 0.504 | 0.716 | 0.301 | 0.486 | 0.670 |
| 60 | 0.200 | 0.490 | 0.780 | 0.208 | 0.430 | 0.652 | 0.460 | 0.637 | 0.814 |
| 61 | 0.296 | 0.598 | 0.900 | 0.195 | 0.417 | 0.638 | 0.235 | 0.413 | 0.592 |
| 62 | 0.297 | 0.598 | 0.900 | 0.206 | 0.425 | 0.645 | 0.360 | 0.538 | 0.717 |
| 63 | 0.303 | 0.588 | 0.874 | 0.343 | 0.557 | 0.771 | 0.359 | 0.546 | 0.733 |
| 64 | 0.549 | 0.765 | 0.980 | 0.372 | 0.588 | 0.803 | 0.323 | 0.507 | 0.692 |
| 65 | 0.286 | 0.569 | 0.851 | 0.443 | 0.649 | 0.855 | 0.440 | 0.613 | 0.786 |
| 66 | 0.167 | 0.441 | 0.715 | 0.341 | 0.561 | 0.782 | 0.355 | 0.558 | 0.760 |
| 67 | 0.262 | 0.588 | 0.914 | 0.529 | 0.741 | 0.953 | 0.432 | 0.620 | 0.809 |
| 68 | 0.310 | 0.608 | 0.906 | 0.375 | 0.588 | 0.800 | 0.366 | 0.548 | 0.731 |
| 69 | 0.348 | 0.637 | 0.926 | 0.315 | 0.544 | 0.773 | 0.411 | 0.591 | 0.772 |
| 70 | 0.450 | 0.745 | 1.000 | 0.546 | 0.741 | 0.936 | 0.551 | 0.709 | 0.868 |
| 71 | 0.384 | 0.647 | 0.911 | 0.471 | 0.671 | 0.871 | 0.480 | 0.656 | 0.833 |
| 72 | 0.228 | 0.539 | 0.851 | 0.342 | 0.553 | 0.764 | 0.415 | 0.591 | 0.768 |
| 73 | 0.496 | 0.735 | 0.975 | 0.356 | 0.575 | 0.793 | 0.307 | 0.498 | 0.688 |
| 74 | 0.222 | 0.559 | 0.896 | 0.397 | 0.623 | 0.848 | 0.376 | 0.575 | 0.773 |
| 75 | 0.390 | 0.667 | 0.943 | 0.355 | 0.570 | 0.785 | 0.263 | 0.445 | 0.626 |
| 76 | 0.176 | 0.471 | 0.766 | 0.509 | 0.702 | 0.894 | 0.348 | 0.536 | 0.724 |
| 77 | 0.357 | 0.627 | 0.898 | 0.553 | 0.741 | 0.930 | 0.415 | 0.594 | 0.772 |
| 78 | 0.255 | 0.549 | 0.843 | 0.370 | 0.596 | 0.823 | 0.483 | 0.663 | 0.844 |
| 79 | 0.264 | 0.588 | 0.912 | 0.382 | 0.610 | 0.837 | 0.440 | 0.627 | 0.815 |
| 80 | 0.306 | 0.598 | 0.890 | 0.413 | 0.623 | 0.832 | 0.344 | 0.524 | 0.704 |
| 81 | 0.301 | 0.598 | 0.895 | 0.490 | 0.697 | 0.905 | 0.428 | 0.606 | 0.783 |
| 82 | 0.338 | 0.608 | 0.878 | 0.325 | 0.539 | 0.754 | 0.436 | 0.620 | 0.804 |
| 83 | 0.244 | 0.569 | 0.893 | 0.302 | 0.522 | 0.742 | 0.339 | 0.522 | 0.704 |
| 84 | 0.224 | 0.569 | 0.913 | 0.367 | 0.579 | 0.791 | 0.403 | 0.579 | 0.756 |
| 85 | 0.357 | 0.608 | 0.859 | 0.347 | 0.557 | 0.767 | 0.345 | 0.524 | 0.703 |
| 86 | 0.214 | 0.549 | 0.884 | 0.432 | 0.645 | 0.857 | 0.401 | 0.584 | 0.768 |
| 87 | 0.303 | 0.618 | 0.932 | 0.305 | 0.539 | 0.774 | 0.313 | 0.498 | 0.682 |
| 88 | 0.340 | 0.647 | 0.954 | 0.378 | 0.601 | 0.824 | 0.380 | 0.575 | 0.769 |
| 89 | 0.472 | 0.735 | 0.999 | 0.598 | 0.781 | 0.964 | 0.411 | 0.591 | 0.772 |
| 90 | 0.401 | 0.637 | 0.873 | 0.381 | 0.592 | 0.803 | 0.372 | 0.553 | 0.734 |
| 91 | 0.499 | 0.716 | 0.933 | 0.221 | 0.434 | 0.648 | 0.344 | 0.531 | 0.718 |
| 92 | 0.469 | 0.725 | 0.982 | 0.334 | 0.548 | 0.762 | 0.308 | 0.498 | 0.687 |
| 93 | 0.348 | 0.608 | 0.868 | 0.346 | 0.570 | 0.794 | 0.324 | 0.517 | 0.710 |
| 94 | 0.318 | 0.559 | 0.800 | 0.446 | 0.645 | 0.843 | 0.470 | 0.639 | 0.809 |
| 95 | 0.414 | 0.657 | 0.900 | 0.412 | 0.632 | 0.851 | 0.381 | 0.565 | 0.749 |
| 96 | 0.645 | 0.824 | 1.000 | 0.346 | 0.566 | 0.786 | 0.369 | 0.558 | 0.746 |
| 97 | 0.308 | 0.657 | 1.000 | 0.357 | 0.583 | 0.810 | 0.436 | 0.611 | 0.785 |
| 98 | 0.278 | 0.578 | 0.879 | 0.588 | 0.776 | 0.965 | 0.468 | 0.659 | 0.850 |
| 99 | 0.364 | 0.647 | 0.930 | 0.381 | 0.596 | 0.812 | 0.422 | 0.596 | 0.771 |
| 100 | 0.445 | 0.696 | 0.947 | 0.371 | 0.592 | 0.813 | 0.414 | 0.603 | 0.793 |
| 101 | 0.276 | 0.559 | 0.841 | 0.365 | 0.596 | 0.828 | 0.317 | 0.505 | 0.692 |
| 102 | 0.353 | 0.608 | 0.863 | 0.330 | 0.557 | 0.784 | 0.411 | 0.603 | 0.795 |
| 103 | 0.367 | 0.667 | 0.966 | 0.408 | 0.623 | 0.838 | 0.417 | 0.596 | 0.775 |
| 104 | 0.439 | 0.706 | 0.973 | 0.294 | 0.513 | 0.732 | 0.421 | 0.594 | 0.767 |
| 105 | 0.288 | 0.598 | 0.908 | 0.353 | 0.570 | 0.787 | 0.351 | 0.529 | 0.707 |
| 106 | 0.270 | 0.549 | 0.828 | 0.297 | 0.526 | 0.755 | 0.403 | 0.591 | 0.779 |
| 107 | 0.415 | 0.676 | 0.938 | 0.320 | 0.539 | 0.759 | 0.276 | 0.457 | 0.637 |
| 108 | 0.333 | 0.618 | 0.903 | 0.273 | 0.496 | 0.718 | 0.364 | 0.543 | 0.722 |
| 109 | 0.256 | 0.618 | 0.979 | 0.254 | 0.482 | 0.711 | 0.435 | 0.611 | 0.786 |
| 110 | 0.196 | 0.451 | 0.706 | 0.323 | 0.531 | 0.739 | 0.361 | 0.538 | 0.716 |
| 111 | 0.185 | 0.539 | 0.894 | 0.355 | 0.583 | 0.811 | 0.448 | 0.632 | 0.817 |
| 112 | 0.255 | 0.559 | 0.862 | 0.353 | 0.583 | 0.813 | 0.413 | 0.606 | 0.798 |
| 113 | 0.343 | 0.578 | 0.814 | 0.476 | 0.671 | 0.866 | 0.420 | 0.594 | 0.768 |
| 114 | 0.336 | 0.578 | 0.821 | 0.393 | 0.601 | 0.809 | 0.411 | 0.589 | 0.767 |
| 115 | 0.269 | 0.559 | 0.848 | 0.353 | 0.570 | 0.787 | 0.370 | 0.553 | 0.735 |
| 116 | 0.365 | 0.667 | 0.969 | 0.299 | 0.513 | 0.728 | 0.363 | 0.541 | 0.719 |
| 117 | 0.258 | 0.549 | 0.840 | 0.356 | 0.566 | 0.775 | 0.501 | 0.668 | 0.835 |
| 118 | 0.321 | 0.627 | 0.934 | 0.390 | 0.614 | 0.838 | 0.364 | 0.553 | 0.742 |
| 119 | 0.367 | 0.627 | 0.888 | 0.511 | 0.697 | 0.884 | 0.498 | 0.673 | 0.849 |
| 120 | 0.264 | 0.578 | 0.893 | 0.503 | 0.706 | 0.909 | 0.618 | 0.764 | 0.911 |
| 121 | 0.323 | 0.637 | 0.952 | 0.344 | 0.566 | 0.788 | 0.352 | 0.538 | 0.725 |
| 122 | 0.203 | 0.510 | 0.817 | 0.376 | 0.583 | 0.791 | 0.423 | 0.599 | 0.774 |
| 123 | 0.219 | 0.500 | 0.781 | 0.348 | 0.579 | 0.810 | 0.358 | 0.555 | 0.753 |
| 124 | 0.223 | 0.569 | 0.914 | 0.282 | 0.509 | 0.736 | 0.274 | 0.457 | 0.639 |
| 125 | 0.174 | 0.539 | 0.905 | 0.325 | 0.539 | 0.754 | 0.428 | 0.601 | 0.774 |
| 126 | 0.249 | 0.549 | 0.849 | 0.357 | 0.583 | 0.809 | 0.482 | 0.659 | 0.836 |
| 127 | 0.165 | 0.471 | 0.776 | 0.288 | 0.526 | 0.764 | 0.296 | 0.490 | 0.685 |
| 128 | 0.336 | 0.618 | 0.900 | 0.463 | 0.662 | 0.861 | 0.468 | 0.644 | 0.820 |
| 129 | 0.188 | 0.510 | 0.832 | 0.246 | 0.474 | 0.701 | 0.348 | 0.546 | 0.743 |
| 130 | 0.263 | 0.549 | 0.835 | 0.367 | 0.588 | 0.808 | 0.468 | 0.647 | 0.826 |
| 131 | 0.269 | 0.578 | 0.888 | 0.271 | 0.504 | 0.738 | 0.408 | 0.599 | 0.789 |
| 132 | 0.407 | 0.676 | 0.945 | 0.343 | 0.561 | 0.780 | 0.379 | 0.565 | 0.750 |
| 133 | 0.446 | 0.676 | 0.907 | 0.370 | 0.583 | 0.796 | 0.332 | 0.526 | 0.721 |
| 134 | 0.246 | 0.539 | 0.832 | 0.371 | 0.588 | 0.804 | 0.436 | 0.615 | 0.795 |
| 135 | 0.281 | 0.578 | 0.876 | 0.378 | 0.610 | 0.841 | 0.420 | 0.611 | 0.801 |
| 136 | 0.243 | 0.569 | 0.895 | 0.284 | 0.539 | 0.795 | 0.298 | 0.502 | 0.706 |
| 137 | 0.227 | 0.549 | 0.871 | 0.340 | 0.575 | 0.810 | 0.421 | 0.613 | 0.805 |
| 138 | 0.285 | 0.559 | 0.833 | 0.431 | 0.645 | 0.859 | 0.428 | 0.611 | 0.793 |
| 139 | 0.416 | 0.667 | 0.917 | 0.286 | 0.500 | 0.714 | 0.282 | 0.462 | 0.641 |
| 140 | 0.186 | 0.510 | 0.834 | 0.430 | 0.662 | 0.894 | 0.403 | 0.599 | 0.794 |
| 141 | 0.040 | 0.363 | 0.686 | 0.327 | 0.548 | 0.770 | 0.407 | 0.591 | 0.776 |
| 142 | 0.268 | 0.539 | 0.811 | 0.386 | 0.605 | 0.825 | 0.325 | 0.519 | 0.713 |
| 143 | 0.136 | 0.451 | 0.766 | 0.341 | 0.566 | 0.791 | 0.346 | 0.543 | 0.740 |
| 144 | 0.351 | 0.637 | 0.924 | 0.329 | 0.553 | 0.776 | 0.270 | 0.466 | 0.663 |
| 145 | 0.208 | 0.520 | 0.832 | 0.331 | 0.561 | 0.792 | 0.328 | 0.519 | 0.710 |
| 146 | 0.570 | 0.784 | 0.999 | 0.217 | 0.447 | 0.677 | 0.325 | 0.519 | 0.714 |
| 147 | 0.191 | 0.510 | 0.829 | 0.369 | 0.596 | 0.824 | 0.390 | 0.579 | 0.769 |
| 148 | 0.174 | 0.490 | 0.806 | 0.403 | 0.614 | 0.825 | 0.426 | 0.606 | 0.786 |
| 149 | 0.410 | 0.716 | 1.000 | 0.249 | 0.469 | 0.689 | 0.429 | 0.603 | 0.778 |
| 150 | 0.346 | 0.627 | 0.909 | 0.499 | 0.702 | 0.905 | 0.374 | 0.563 | 0.751 |
| 151 | 0.302 | 0.627 | 0.953 | 0.337 | 0.575 | 0.812 | 0.296 | 0.481 | 0.666 |
| 152 | 0.352 | 0.657 | 0.962 | 0.307 | 0.526 | 0.746 | 0.270 | 0.452 | 0.634 |
| 153 | 0.493 | 0.775 | 1.000 | 0.383 | 0.592 | 0.801 | 0.494 | 0.663 | 0.833 |
| 154 | 0.402 | 0.686 | 0.971 | 0.240 | 0.461 | 0.682 | 0.534 | 0.700 | 0.865 |
| 155 | 0.490 | 0.755 | 1.000 | 0.360 | 0.575 | 0.789 | 0.542 | 0.704 | 0.866 |
| 156 | 0.404 | 0.686 | 0.969 | 0.336 | 0.548 | 0.760 | 0.541 | 0.707 | 0.872 |
| 157 | 0.372 | 0.676 | 0.981 | 0.243 | 0.465 | 0.687 | 0.381 | 0.563 | 0.744 |
| 158 | 0.465 | 0.745 | 1.000 | 0.241 | 0.456 | 0.671 | 0.556 | 0.716 | 0.877 |
| 159 | 0.475 | 0.755 | 1.000 | 0.346 | 0.557 | 0.768 | 0.521 | 0.688 | 0.854 |
| 160 | 0.434 | 0.716 | 0.997 | 0.425 | 0.640 | 0.855 | 0.516 | 0.688 | 0.859 |
| 161 | 0.421 | 0.706 | 0.990 | 0.357 | 0.566 | 0.775 | 0.516 | 0.683 | 0.850 |
| 162 | 0.509 | 0.725 | 0.942 | 0.341 | 0.566 | 0.791 | 0.391 | 0.575 | 0.758 |
| 163 | 0.187 | 0.549 | 0.911 | 0.261 | 0.487 | 0.713 | 0.399 | 0.584 | 0.770 |
| 164 | 0.403 | 0.686 | 0.969 | 0.400 | 0.610 | 0.819 | 0.505 | 0.673 | 0.841 |
| 165 | 0.419 | 0.686 | 0.954 | 0.351 | 0.561 | 0.772 | 0.507 | 0.678 | 0.849 |
| 166 | 0.411 | 0.696 | 0.981 | 0.375 | 0.588 | 0.801 | 0.478 | 0.654 | 0.829 |
| 167 | 0.334 | 0.637 | 0.940 | 0.542 | 0.728 | 0.915 | 0.433 | 0.606 | 0.779 |
| 168 | 0.420 | 0.696 | 0.972 | 0.344 | 0.553 | 0.761 | 0.517 | 0.683 | 0.848 |
| 169 | 0.324 | 0.598 | 0.872 | 0.363 | 0.575 | 0.786 | 0.466 | 0.639 | 0.813 |
| 170 | 0.466 | 0.696 | 0.927 | 0.479 | 0.675 | 0.872 | 0.497 | 0.675 | 0.854 |
| 171 | 0.197 | 0.529 | 0.861 | 0.453 | 0.654 | 0.854 | 0.460 | 0.637 | 0.814 |
| 172 | 0.444 | 0.725 | 1.000 | 0.241 | 0.456 | 0.671 | 0.562 | 0.719 | 0.876 |
| 173 | 0.232 | 0.510 | 0.788 | 0.343 | 0.566 | 0.788 | 0.279 | 0.459 | 0.640 |
| 174 | 0.392 | 0.667 | 0.941 | 0.269 | 0.482 | 0.696 | 0.527 | 0.695 | 0.862 |
| 175 | 0.233 | 0.539 | 0.845 | 0.258 | 0.487 | 0.716 | 0.378 | 0.567 | 0.757 |
| 176 | 0.372 | 0.676 | 0.981 | 0.236 | 0.456 | 0.676 | 0.538 | 0.707 | 0.875 |
| 177 | 0.164 | 0.500 | 0.836 | 0.351 | 0.575 | 0.799 | 0.442 | 0.623 | 0.803 |
| 178 | 0.331 | 0.608 | 0.884 | 0.260 | 0.474 | 0.687 | 0.511 | 0.678 | 0.844 |
| 179 | 0.418 | 0.686 | 0.954 | 0.338 | 0.548 | 0.758 | 0.530 | 0.695 | 0.859 |
| 180 | 0.158 | 0.451 | 0.744 | 0.203 | 0.417 | 0.631 | 0.372 | 0.558 | 0.743 |
| 181 | 0.416 | 0.696 | 0.976 | 0.327 | 0.539 | 0.752 | 0.516 | 0.685 | 0.854 |
| 182 | 0.383 | 0.647 | 0.911 | 0.296 | 0.509 | 0.722 | 0.563 | 0.724 | 0.884 |
| 183 | 0.350 | 0.627 | 0.905 | 0.371 | 0.583 | 0.796 | 0.516 | 0.685 | 0.855 |
| 184 | 0.441 | 0.735 | 1.000 | 0.328 | 0.561 | 0.795 | 0.355 | 0.541 | 0.727 |
| 185 | 0.432 | 0.735 | 1.000 | 0.300 | 0.539 | 0.779 | 0.329 | 0.517 | 0.705 |
| 186 | 0.309 | 0.618 | 0.926 | 0.261 | 0.491 | 0.721 | 0.324 | 0.510 | 0.695 |
| 187 | 0.297 | 0.559 | 0.821 | 0.237 | 0.461 | 0.685 | 0.307 | 0.498 | 0.688 |
| 188 | 0.394 | 0.667 | 0.939 | 0.326 | 0.539 | 0.753 | 0.574 | 0.733 | 0.892 |
| 189 | 0.282 | 0.598 | 0.914 | 0.521 | 0.706 | 0.891 | 0.470 | 0.647 | 0.823 |
| 190 | 0.397 | 0.696 | 0.995 | 0.376 | 0.592 | 0.809 | 0.512 | 0.680 | 0.849 |
| 191 | 0.206 | 0.569 | 0.932 | 0.362 | 0.588 | 0.813 | 0.419 | 0.601 | 0.783 |
| 192 | 0.404 | 0.686 | 0.969 | 0.333 | 0.544 | 0.755 | 0.518 | 0.685 | 0.852 |
| 193 | 0.223 | 0.569 | 0.914 | 0.477 | 0.671 | 0.865 | 0.459 | 0.635 | 0.810 |
| 194 | 0.234 | 0.539 | 0.845 | 0.420 | 0.632 | 0.843 | 0.432 | 0.611 | 0.789 |
| 195 | 0.218 | 0.578 | 0.939 | 0.408 | 0.623 | 0.838 | 0.446 | 0.627 | 0.809 |
| 196 | 0.343 | 0.637 | 0.931 | 0.334 | 0.548 | 0.762 | 0.398 | 0.575 | 0.751 |
| 197 | 0.261 | 0.569 | 0.877 | 0.400 | 0.614 | 0.828 | 0.396 | 0.579 | 0.763 |
| 198 | 0.461 | 0.725 | 0.990 | 0.317 | 0.535 | 0.753 | 0.528 | 0.695 | 0.862 |
| 199 | 0.440 | 0.725 | 1.000 | 0.335 | 0.561 | 0.788 | 0.506 | 0.683 | 0.860 |
| 200 | 0.148 | 0.520 | 0.891 | 0.328 | 0.557 | 0.786 | 0.457 | 0.635 | 0.812 |

TABLE 17
Predictors that passed verification in at least one GABD window

| | | | | Discovery | Verification | | | | | | | | |
|---|---|---|---|---|---|---|---|---|---|---|---|---|---|
| | Univariate/ | | | GABD | GABD | Discovery Mean AUC | | | | Verfication Mean AUC | | | |
| Biomarker(s) | Bivariate | Numerator | Denominator | Window | Window | Full | Early | Middle | Late | Full | Early | Middle | Late |
| 1 | Bivariate | hsa-miR-127-3p | hsa-miR-485-5p | Full, | Full | 0.660 | 0.571 | 0.613 | 0.700 | 0.670 | 0.696 | 0.675 | 0.675 |
| | | | | Late | | | | | | | | | |
| 2 | Bivariate | hsa-miR-26b-5p | hsa-miR-485-5p | Full, | Middle | 0.671 | 0.615 | 0.687 | 0.677 | 0.585 | 0.598 | 0.706 | 0.647 |
| | | | | Late | | | | | | | | | |
| 3 | Bivariate | hsa-miR-98-5p | hsa-miR-485-5p | Full, | Middle | 0.680 | 0.613 | 0.685 | 0.691 | 0.581 | 0.637 | 0.728 | 0.606 |
| | | | | Late | | | | | | | | | |
| 4 | Bivariate | hsa-miR-182-5p | hsa-miR-485-5p | Full | Late | 0.666 | 0.680 | 0.594 | 0.650 | 0.623 | 0.461 | 0.728 | 0.712 |
| 5 | Bivariate | hsa-miR-4732-3p | hsa-miR-941 | Early | Early | 0.553 | 0.809 | 0.573 | 0.580 | 0.526 | 0.804 | 0.632 | 0.680 |
| 6 | Bivariate | hsa-miR-7-5p | hsa-miR-941 | Early | Middle | 0.654 | 0.777 | 0.681 | 0.599 | 0.609 | 0.588 | 0.741 | 0.620 |
| 7 | Bivariate | hsa-miR-1273h-3p | hsa-miR-3173-5p | Early | Early | 0.571 | 0.832 | 0.574 | 0.532 | 0.620 | 0.824 | 0.566 | 0.558 |
| 8 | Bivariate | hsa-miR-155-5p | hsa-miR-3173-5p | Early | Early | 0.561 | 0.762 | 0.551 | 0.501 | 0.582 | 0.765 | 0.588 | 0.507 |
| 9 | Bivariate | hsa-miR-150-3p | hsa-miR-193b-5p | Middle | Full, | 0.634 | 0.759 | 0.731 | 0.583 | 0.700 | 0.578 | 0.706 | 0.764 |
| 10 | Bivariate | hsa-miR-378e | hsa-miR-221-5p | Early | Middle | 0.621 | 0.734 | 0.700 | 0.574 | 0.585 | 0.735 | 0.781 | 0.591 |
| 11 | Bivariate | hsa-miR-1285-3p | hsa-mir-378c | Middle | Middle | 0.564 | 0.613 | 0.704 | 0.536 | 0.652 | 0.627 | 0.697 | 0.673 |
| 12 | Bivariate | hsa-miR-4732-3p | hsa-miR-381-3p | Early | Early | 0.530 | 0.819 | 0.633 | 0.575 | 0.504 | 0.804 | 0.456 | 0.560 |
| 13 | Bivariate | hsa-miR-345-5p | hsa-miR-324-3p | Late | Early | 0.569 | 0.692 | 0.572 | 0.679 | 0.515 | 0.725 | 0.566 | 0.575 |
| 14 | Bivariate | hsa-miR-320b | hsa-miR-155-5p | Late | Late | 0.579 | 0.618 | 0.603 | 0.695 | 0.543 | 0.696 | 0.592 | 0.680 |
| 15 | Bivariate | hsa-miR-181a-5p | hsa-miR-155-5p | Late | Late | 0.591 | 0.583 | 0.588 | 0.702 | 0.521 | 0.696 | 0.539 | 0.685 |
| 16 | Bivariate | hsa-miR-26b-5p | hsa-miR-155-5p | Late | Late | 0.594 | 0.529 | 0.607 | 0.685 | 0.548 | 0.647 | 0.509 | 0.724 |
| 17 | Bivariate | hsa-let-7g-5p | hsa-miR-155-5p | Late | Late | 0.587 | 0.583 | 0.597 | 0.703 | 0.529 | 0.686 | 0.561 | 0.678 |
| 18 | Bivariate | hsa-miR-4443 | hsa-miR-155-5p | Late | Late | 0.571 | 0.643 | 0.556 | 0.716 | 0.529 | 0.725 | 0.561 | 0.683 |
| 19 | Bivariate | hsa-miR-425-5p | hsa-miR-155-5p | Late | Late | 0.579 | 0.598 | 0.576 | 0.696 | 0.543 | 0.686 | 0.544 | 0.685 |
| 20 | Bivariate | hsa-miR-146a-5p | hsa-miR-155-5p | Late | Late | 0.570 | 0.630 | 0.543 | 0.701 | 0.570 | 0.608 | 0.474 | 0.678 |
| 21 | Bivariate | hsa-miR-25-3p | hsa-miR-155-5p | Late | Late | 0.593 | 0.571 | 0.570 | 0.706 | 0.525 | 0.755 | 0.557 | 0.688 |
| 22 | Bivariate | hsa-miR-151a-3p | hsa-miR-155-5p | Late | Late | 0.582 | 0.628 | 0.565 | 0.711 | 0.535 | 0.696 | 0.553 | 0.683 |
| 23 | Bivariate | hsa-miR-320a | hsa-miR-155-5p | Late | Late | 0.579 | 0.615 | 0.607 | 0.709 | 0.541 | 0.686 | 0.610 | 0.673 |
| 24 | Bivariate | hsa-miR-30d-5p | hsa-miR-155-5p | Late | Late | 0.579 | 0.640 | 0.564 | 0.710 | 0.537 | 0.706 | 0.566 | 0.683 |
| 25 | Bivariate | hsa-miR-126-3p | hsa-miR-155-5p | Late | Late | 0.578 | 0.618 | 0.569 | 0.702 | 0.551 | 0.667 | 0.539 | 0.733 |
| 26 | Bivariate | hsa-miR-146b-5p | hsa-miR-155-5p | Late | Late | 0.574 | 0.638 | 0.550 | 0.702 | 0.558 | 0.627 | 0.583 | 0.685 |
| 27 | Bivariate | hsa-let-7i-5p | hsa-miR-155-5p | Late | Late | 0.600 | 0.603 | 0.607 | 0.736 | 0.552 | 0.686 | 0.548 | 0.707 |
| 28 | Bivariate | hsa-miR-26a-5p | hsa-miR-155-5p | Late | Late | 0.562 | 0.648 | 0.561 | 0.697 | 0.536 | 0.686 | 0.548 | 0.695 |
| 29 | Bivariate | hsa-miR-625-3p | hsa-miR-155-5p | Late | Late | 0.583 | 0.638 | 0.588 | 0.730 | 0.536 | 0.667 | 0.482 | 0.695 |
| 30 | Bivariate | hsa-miR-423-5p | hsa-miR-155-5p | Late | Late | 0.586 | 0.596 | 0.586 | 0.698 | 0.553 | 0.725 | 0.456 | 0.719 |
| 31 | Bivariate | hsa-miR-451a | hsa-miR-155-5p | Late | Late | 0.608 | 0.571 | 0.603 | 0.718 | 0.543 | 0.686 | 0.461 | 0.700 |
| 32 | Bivariate | hsa-miR-125a-5p | hsa-miR-155-5p | Late | Late | 0.585 | 0.625 | 0.611 | 0.714 | 0.512 | 0.755 | 0.575 | 0.704 |
| 33 | Bivariate | hsa-miR-99a-5p | hsa-miR-155-5p | Late | Late | 0.579 | 0.603 | 0.557 | 0.696 | 0.559 | 0.676 | 0.456 | 0.707 |
| 34 | Bivariate | hsa-let-7b-5p | hsa-miR-155-5p | Late | Late | 0.596 | 0.596 | 0.611 | 0.713 | 0.540 | 0.745 | 0.456 | 0.732 |
| 35 | Bivariate | hsa-miR-516b-5p | hsa-miR-155-5p | Late | Late | 0.545 | 0.720 | 0.474 | 0.699 | 0.537 | 0.716 | 0.640 | 0.688 |
| 36 | Bivariate | hsa-miR-363-3p | hsa-miR-155-5p | Late | Late | 0.593 | 0.581 | 0.606 | 0.699 | 0.526 | 0.725 | 0.535 | 0.695 |
| 37 | Univariate | hsa-miR-331-3p | | Late | Full | 0.562 | 0.414 | 0.537 | 0.621 | 0.677 | 0.676 | 0.610 | 0.663 |
| 38 | Univariate | hsa-miR-4732-5p | | Middle | Early | 0.507 | 0.618 | 0.684 | 0.540 | 0.610 | 0.745 | 0.583 | 0.555 |
| 39 | Univariate | hsa-miR-1273h-3p | | Late | Early | 0.546 | 0.615 | 0.504 | 0.624 | 0.563 | 0.745 | 0.496 | 0.541 |
| 40 | Univariate | hsa-miR-516b-5p | | Early, | Early, | 0.489 | 0.809 | 0.641 | 0.591 | 0.544 | 0.755 | 0.697 | 0.522 |
| | | | | Late | Middle | | | | | | | | |
| 41 | Univariate | hsa-miR-941 | | Middle, | Middle | 0.645 | 0.690 | 0.698 | 0.645 | 0.560 | 0.490 | 0.732 | 0.623 |
| | | | | Full | | | | | | | | | |
| | Discovery | | Verification | | | | | | | | | | |
| | Expression of | Expression of | | Expression of | | | | | | | | | |
| | individual | individual | Expression of | individual | | | | | | | | | |
| | miRNAs: | miRNAs | individual | miRNAs | | | | | Tissue Atlas | | | | |
| | Direction | (case vs ctrl): | miRNAs: | (case vs ctrl): | | | | | Numerator | Associated Carrier | | Overlap with | |
| | (case vs | Wilcoxon | Direction | Wilcoxon | Chromosome | | In miRNA Cluster | | Max/(Max- | Subclasses | | Literature | |
| Biomarker(s) | ctrl) | p-value | (case vs ctrl) | p-value | Numerator | Denominator | Numerator | Denominator | 10%) | Numerator | Denominator | Numerator | Denominator |
| 1 | Down/ | 0.926/ | Up/ | 0.599/ | chr14 | chr14 | Y | Y | Placenta/ | CD63 | | | |
| | Down | 0.001 | Down | 0.228 | | | | | Liver | | | | |
| 2 | Up/ | 0.698/ | Up/ | 0.13/ | chr02 | chr14 | Y | | Liver/ | PLAP | | | |
| | Down | 0.001 | Down | 0.152 | | | | | Placenta | | | | |
| 3 | Up/ | 0.824/ | Up/ | 0.252/ | chrX | chr14 | Y | Y | Platelets/ | CD63 | | | |
| | Down | 0.001 | Down | 0.152 | | | | | RBC | | | | |
| 4 | Up/ | 0.737/ | Up/ | 0.53/ | chr07 | chr14 | Y | Y | RBC | | | | |
| | Down | 0.001 | Down | 0.176 | | | | | | | | | |
| 5 | Up/ | 0.095/ | Down/ | 0.074/ | chr17 | chr20 | Y | Y | RBC | Input_ | Input_CD63 | | |
| | Down | 0.05 | Down | 0.973 | | | | | | AGO2 | | | |
| 6 | Down/ | 0.23/ | Up/ | 0.435/ | chr15\| | chr20 | Y | Y | Brain | | Input_CD63 | | |
| | Down | 0.05 | Down | 0.032 | chr19\| | | | | | | | | |
| | | | | | chr09 | | | | | | | | |
| 7 | Up/Up | 0.24/ | Up/ | 0.087/ | chr16 | chr14 | | | Platelets | | | | |
| | | 0.576 | Down | 0.319 | | | | | | | | | |
| 8 | Up/Up | 0.401/ | Up/ | 0.087/ | chr21 | chr14 | | | Lymphocytes | | | Jairajpuri | |
| | | 0.576 | Down | 0.319 | | | | | | | | 2017up | |
| 9 | Up/Up | 0.751/ | Down/ | 0.617/ | chr19 | chr16 | | Y | Lymphocytes | | | | |
| | | 0.988 | Down | 0.326 | | | | | | | | | |
| 10 | Down/ | 0.8/ | Down/ | 0.535/ | chr05 | chrX | | Y | Liver | | CD63 | | |
| | Up | 0.611 | Up | 0.002 | | | | | | | | | |
| 11 | Up/Up | 0.213/ | Down/ | 0.795/ | chr02\| | chr10 | | | RBC | Input_ | Input | | |
| | | 0.288 | Up | 0.562 | chr07 | | | | | AGO2 | | | |
| 12 | Up/ | 0.095/ | Down/ | 0.074/ | chr17 | chr14 | Y | Y | RBC | Input_ | | | |
| | Down | 0.192 | Up | 0.177 | | | | | | AGO2 | | | |
| 13 | Down/ | 0.073/ | Down/ | 0.473/1 | chr14 | chr17 | | | Liver | Input | | | |
| | Down | 0.05 | Up | | | | | | | | | | |
| 14 | Up/ | 0.241/ | Up/ | 0.673/ | chr01 | chr21 | | | Liver/RBC | PLAP | | | Jairajpuri |
| | Down | 0.001 | Down | 0.1 | | | | | | | | | 2017up |
| 15 | Up/ | 0.231/ | Up/ | 0.691/ | chr01\| | chr21 | | | Placenta | | | | Jairajpuri |
| | Down | 0.001 | Down | 0.1 | chr09 | | | | | | | | 2017up |
| 16 | Up/ | 0.75/ | Down/ | 0.888/ | chr02 | chr21 | | | Liver/ | PLAP | | | Jairajpuri |
| | Down | 0.001 | Down | 0.1 | | | | | Placenta | | | | 2017up |
| 17 | Down/ | 0.821/ | Down/ | 0.286/ | chr03 | chr21 | | | RBC | CD63 | | Yoffe | Jairajpuri |
| | Down | 0.001 | Down | 0.1 | | | | | | | | 2017up | 2017up |
| 18 | Up/ | 0.255/ | Up/ | 0.908/ | chr03 | chr21 | | | Liver | PLAP | | | Jairajpuri |
| | Down | 0.001 | Down | 0.1 | | | | | | | | | 2017up |
| 19 | Up/ | 0.276/ | Down/ | 0.828/ | chr03 | chr21 | Y | | Platelets/ | CD63 | | | Jairajpuri |
| | Down | 0.001 | Down | 0.1 | | | | | Placenta/ | | | | 2017up |
| | | | | | | | | | Liver/ | | | | |
| | | | | | | | | | RBC | | | | |
| 20 | Up/ | 0.75/ | Up/ | 0.599/ | chr05 | chr21 | | | Platelets | Input_C | | | Jairajpuri |
| | Down | 0.001 | Down | 0.1 | | | | | | D63 | | | 2017up |
| 21 | Up/ | 0.795/ | Down/ | 0.828/ | chr07 | chr21 | Y | | RBC | Input | | Yoffe2017 | Jairajpuri |
| | Down | 0.001 | Down | 0.1 | | | | | | | | down | 2017up |
| 22 | Down/ | 0.61/ | Down/ | 0.888/ | chr08 | chr21 | | | Platelets | CD63 | | Wu2012up | Jairajpuri |
| | Down | 0.001 | Down | 0.1 | | | | | | | | | 2017up |
| 23 | Up/ | 0.88/ | Up/ | 0.868/ | chr08 | chr21 | | | Placenta/ | PLAP | | | Jairajpuri |
| | Down | 0.001 | Down | 0.1 | | | | | Liver | | | | 2017up |
| 24 | Down/ | 0.887/ | Down/ | 0.808/ | chr08 | chr21 | Y | | Placenta | PLAP | | | Jairajpuri |
| | Down | 0.001 | Down | 0.1 | | | | | | | | | 2017up |
| 25 | Down/ | 0.34/ | Down/ | 0.908/ | chr09 | chr21 | | | Platelets | CD63 | | Salomon | Jairajpuri |
| | Down | 0.001 | Down | 0.1 | | | | | | | | 2017down | 2017up |
| 26 | Down/ | 0.604/ | Up/ | 0.768/ | chr10 | chr21 | | | Liver | CD63 | | Yoffe2017 | Jairajpuri |
| | Down | 0.001 | Down | 0.1 | | | | | | | | down | 2017up |
| 27 | Up/ | 0.388/ | Up/ | 0.969/ | chr12 | chr21 | | | RBC | CD63 | | | Jairajpuri |
| | Down | 0.001 | Down | 0.1 | | | | | | | | | 2017up |
| 28 | Down/ | 0.262/ | Down/ | 0.73/ | chr12\| | chr21 | | | Platelets/ | CD63 | | | Jairajpuri |
| | Down | 0.001 | Down | 0.1 | chr03 | | | | Liver/ | | | | 2017up |
| | | | | | | | | | Placenta | | | | |
| 29 | Up/ | 0.808/ | Down/ | 0.564/ | chr14 | chr21 | | | Platelets | CD63 | | | Jairajpuri |
| | Down | 0.001 | Down | 0.1 | | | | | | | | | 2017up |
| 30 | Down/ | 0.789/ | Up/ | 0.286/ | chr17 | chr21 | Y | | Platelets | AGO2 | | Timofeeva | Jairajpuri |
| | Down | 0.001 | Down | 0.1 | | | | | | | | 2018up, | 2017up |
| | | | | | | | | | | | | Salomon | |
| | | | | | | | | | | | | 2017up | |
| 31 | Up/ | 0.431/ | Down/ | 0.53/ | chr17 | chr21 | Y | | RBC | AGO2 | | Salomon | Jairajpuri |
| | Down | 0.001 | Down | 0.1 | | | | | | | | 2017up | 2017up |
| 32 | Up/ | 0.215/ | Down/ | 0.39/ | chr19 | chr21 | Y | | Placenta | PLAP | | Yang | Jairajpuri |
| | Down | 0.001 | Down | 0.1 | | | | | | | | 2011up | 2017up |
| 33 | Up/ | 0.947/ | Up/ | 0.08/ | chr21 | chr21 | Y | | Liver | | | | Jairajpuri |
| | Down | 0.001 | Down | 0.1 | | | | | | | | | 2017up |
| 34 | Up/ | 0.28/ | Down/ | 0.691/ | chr22 | chr21 | Y | | RBC | | | | Jairajpuri |
| | Down | 0.001 | Down | 0.1 | | | | | | | | | 2017up |
| 35 | Up/ | 0.152/ | Up/ | 0.828/ | chr19 | chr21 | Y | | Placenta | PLAP | | Hromad | Jairajpuri |
| | Down | 0.001 | Down | 0.1 | | | | | | | | nikova | 2017up |
| | | | | | | | | | | | | 2013up, | |
| | | | | | | | | | | | | Miura | |
| | | | | | | | | | | | | 2015up | |
| 36 | Up/ | 0.558/ | Up/ | 1/0.1 | chrX | chr21 | Y | | RBC | | | | Jairajpuri |
| | Down | 0.001 | Down | | | | | | | | | | 2017up |
| 37 | Down | 0.056 | Down | 0.015 | chr12 | | Y | | Liver | | | | |
| 38 | Up | 0.021 | Down | 0.087 | chr17 | | Y | | RBC | | | | |
| 39 | Down | 0.051 | Up | 0.087 | chr16 | | | | Platelets | | | | |
| 40 | Down | 0.001 | Down | 0.074 | chr19 | | Y | | Placenta | PLAP | | Hromad | |
| | | | | | | | | | | | | nikova | |
| | | | | | | | | | | | | 2013up, | |
| | | | | | | | | | | | | Miura | |
| | | | | | | | | | | | | 2015up | |
| 41 | Down | 0.005 | Down | 0.032 | chr20 | | Y | | Liver | Input_CD63 | | | |

TABLE 18
Univariate predictors and reversals selected in discovery and confirmed in blinded verification

| | | | | | | | | | | | | | | miRNA | | Tissue Atlas | | Carrier | |
|---|---|---|---|---|---|---|---|---|---|---|---|---|---|---|---|---|---|---|---|
| Univariate/ | | | GABD | Discovery Mean AUC | | | | Verification Mean AUC | | | | Chromosome | | Cluster | | (Max/(Max-10%)) | | Subclasses | |
| Bivariate | Numerator | Denominator | Window | Full | Early | Middle | Late | Full | Early | Middle | Late | Num | Denom | Num | Denom | Num | Denom | Num | Denom |
| Bivariate | hsa-miR- | hsa-miR- | Full | 0.66 | 0.57 | 0.61 | 0.70 | 0.67 | 0.70 | 0.68 | 0.68 | chr14 | chr14 | Y | Y | Placenta/Liver | Platelets | CD63 | |
| | 127-3p | 485-5p | | | | | | | | | | | | | | | | | |
| Bivariate | hsa-miR- | hsa-miR- | Early | 0.55 | 0.81 | 0.57 | 0.58 | 0.53 | 0.80 | 0.63 | 0.68 | chr17 | chr20 | Y | Y | RBC | Liver | Input_AGO2 | Input_CD63 |
| | 4732-3p | 941 | | | | | | | | | | | | | | | | | |
| Bivariate | hsa-miR- | hsa-miR- | Early | 0.57 | 0.83 | 0.57 | 0.53 | 0.62 | 0.82 | 0.57 | 0.56 | chr16 | chr14 | | | Platelets | Platelets | | |
| | 1273h-3p | 3173-5p | | | | | | | | | | | | | | | | | |
| Bivariate | hsa-miR- | hsa-miR- | Early | 0.56 | 0.76 | 0.55 | 0.50 | 0.58 | 0.76 | 0.59 | 0.51 | chr21 | chr14 | | | Lymphocytes | Platelets | | |
| | 155-5p | 3173-5p | | | | | | | | | | | | | | | | | |
| Bivariate | hsa-miR- | hsa-miR- | Middle | 0.63 | 0.76 | 0.73 | 0.58 | 0.70 | 0.58 | 0.71 | 0.76 | chr19 | chr16 | | Y | Lymphocytes | Liver | | |
| | 150-3p | 193b-5p | | | | | | | | | | | | | | | | | |
| Bivariate | hsa-miR- | hsa-mir- | Middle | 0.56 | 0.61 | 0.70 | 0.54 | 0.65 | 0.63 | 0.70 | 0.67 | chr02\| | chr10 | | | RBC | Liver | Input_AGO2 | Input |
| | 1285-3p | 378c | | | | | | | | | | chr07 | | | | | | | |
| Bivariate | hsa-miR- | hsa-miR- | Early | 0.53 | 0.82 | 0.63 | 0.57 | 0.50 | 0.80 | 0.46 | 0.56 | chr17 | chr14 | Y | Y | RBC | Placenta | Input_AGO2 | |
| | 4732-3p | 381-3p | | | | | | | | | | | | | | | | | |
| Bivariate | hsa-miR- | hsa-miR- | Late | 0.58 | 0.62 | 0.60 | 0.70 | 0.54 | 0.70 | 0.59 | 0.68 | chr01 | chr21 | | | Liver/RBC | Lymphocytes | PLAP | |
| | 320b | 155-5p | | | | | | | | | | | | | | | | | |
| Bivariate | hsa-miR- | hsa-miR- | Late | 0.59 | 0.58 | 0.59 | 0.70 | 0.52 | 0.70 | 0.54 | 0.69 | chr01\| | chr21 | | | Placenta | Lymphocytes | | |
| | 181a-5p | 155-5p | | | | | | | | | | chr09 | | | | | | | |
| Bivariate | hsa-miR- | hsa-miR- | Late | 0.59 | 0.53 | 0.61 | 0.69 | 0.55 | 0.65 | 0.51 | 0.72 | chr02 | chr21 | | | Liver/Placenta | Lymphocytes | PLAP | |
| | 26b-5p | 155-5p | | | | | | | | | | | | | | | | | |
| Bivariate | hsa-let-7g- | hsa-miR- | Late | 0.59 | 0.58 | 0.60 | 0.70 | 0.53 | 0.69 | 0.56 | 0.68 | chr03 | chr21 | | | RBC | Lymphocytes | CD63 | |
| | 5p | 155-5p | | | | | | | | | | | | | | | | | |
| Bivariate | hsa-miR- | hsa-miR- | Late | 0.57 | 0.64 | 0.56 | 0.72 | 0.53 | 0.73 | 0.56 | 0.68 | chr03 | chr21 | | | Liver | Lymphocytes | PLAP | |
| | 4443 | 155-5p | | | | | | | | | | | | | | | | | |
| Bivariate | hsa-miR- | hsa-miR- | Late | 0.58 | 0.60 | 0.58 | 0.70 | 0.54 | 0.69 | 0.54 | 0.69 | chr03 | chr21 | Y | | Platelets/Placenta/ | Lymphocytes | CD63 | |
| | 425-5p | 155-5p | | | | | | | | | | | | | | Liver/RBC | | | |
| Bivariate | hsa-miR- | hsa-miR- | Late | 0.57 | 0.63 | 0.54 | 0.70 | 0.57 | 0.61 | 0.47 | 0.68 | chr05 | chr21 | | | Platelets | Lymphocytes | Input_CD63 | |
| | 146a-5p | 155-5p | | | | | | | | | | | | | | | | | |
| Bivariate | hsa-miR- | hsa-miR- | Late | 0.59 | 0.57 | 0.57 | 0.71 | 0.52 | 0.75 | 0.56 | 0.69 | chr07 | chr21 | Y | | RBC | Lymphocytes | Input | |
| | 25-3p | 155-5p | | | | | | | | | | | | | | | | | |
| Bivariate | hsa-miR- | hsa-miR- | Late | 0.58 | 0.63 | 0.57 | 0.71 | 0.54 | 0.70 | 0.55 | 0.68 | chr08 | chr21 | | | Platelets | Lymphocytes | CD63 | |
| | 151a-3p | 155-5p | | | | | | | | | | | | | | | | | |
| Bivariate | hsa-miR- | hsa-miR- | Late | 0.58 | 0.62 | 0.61 | 0.71 | 0.54 | 0.69 | 0.61 | 0.67 | chr08 | chr21 | | | Placenta/Liver | Lymphocytes | PLAP | |
| | 320a | 155-5p | | | | | | | | | | | | | | | | | |
| Bivariate | hsa-miR- | hsa-miR- | Late | 0.58 | 0.64 | 0.56 | 0.71 | 0.54 | 0.71 | 0.57 | 0.68 | chr08 | chr21 | Y | | Placenta | Lymphocytes | PLAP | |
| | 30d-5p | 155-5p | | | | | | | | | | | | | | | | | |
| Bivariate | hsa-miR- | hsa-miR- | Late | 0.58 | 0.62 | 0.57 | 0.70 | 0.55 | 0.67 | 0.54 | 0.73 | chr09 | chr21 | | | Platelets | Lymphocytes | CD63 | |
| | 126-3p | 155-5p | | | | | | | | | | | | | | | | | |
| Bivariate | hsa-miR- | hsa-miR- | Late | 0.57 | 0.64 | 0.55 | 0.70 | 0.56 | 0.63 | 0.58 | 0.69 | chr10 | chr21 | | | Liver | Lymphocytes | CD63 | |
| | 146b-5p | 155-5p | | | | | | | | | | | | | | | | | |
| Bivariate | hsa-let-7i- | hsa-miR- | Late | 0.60 | 0.60 | 0.61 | 0.74 | 0.55 | 0.69 | 0.55 | 0.71 | chr12 | chr21 | | | RBC | Lymphocytes | CD63 | |
| | 5p | 155-5p | | | | | | | | | | | | | | | | | |
| Bivariate | hsa-miR- | hsa-miR- | Late | 0.56 | 0.65 | 0.56 | 0.70 | 0.54 | 0.69 | 0.55 | 0.69 | chr12\| | chr21 | | | Platelets/Liver/ | Lymphocytes | CD63 | |
| | 26a-5p | 155-5p | | | | | | | | | | chr03 | | | | Placenta | | | |
| Bivariate | hsa-miR- | hsa-miR- | Late | 0.58 | 0.64 | 0.59 | 0.73 | 0.54 | 0.67 | 0.48 | 0.69 | chr14 | chr21 | | | Platelets | Lymphocytes | CD63 | |
| | 625-3p | 155-5p | | | | | | | | | | | | | | | | | |
| Bivariate | hsa-miR- | hsa-miR- | Late | 0.59 | 0.60 | 0.59 | 0.70 | 0.55 | 0.73 | 0.46 | 0.72 | chr17 | chr21 | Y | | Platelets | Lymphocytes | AGO2 | |
| | 423-5p | 155-5p | | | | | | | | | | | | | | | | | |
| Bivariate | hsa-miR- | hsa-miR- | Late | 0.61 | 0.57 | 0.60 | 0.72 | 0.54 | 0.69 | 0.46 | 0.70 | chr17 | chr21 | Y | | RBC | Lymphocytes | AGO2 | |
| | 451a | 155-5p | | | | | | | | | | | | | | | | | |
| Bivariate | hsa-miR- | hsa-miR- | Late | 0.58 | 0.63 | 0.61 | 0.71 | 0.51 | 0.75 | 0.57 | 0.70 | chr19 | chr21 | Y | | Placenta | Lymphocytes | PLAP | |
| | 125a-5p | 155-5p | | | | | | | | | | | | | | | | | |
| Bivariate | hsa-miR- | hsa-miR- | Late | 0.58 | 0.60 | 0.56 | 0.70 | 0.56 | 0.68 | 0.46 | 0.71 | chr21 | chr21 | Y | | Liver | Lymphocytes | | |
| | 99a-5p | 155-5p | | | | | | | | | | | | | | | | | |
| Bivariate | hsa-let-7b- | hsa-miR- | Late | 0.60 | 0.60 | 0.61 | 0.71 | 0.54 | 0.75 | 0.46 | 0.73 | chr22 | chr21 | Y | | RBC | Lymphocytes | | |
| | 5p | 155-5p | | | | | | | | | | | | | | | | | |
| Bivariate | hsa-miR- | hsa-miR- | Late | 0.55 | 0.72 | 0.47 | 0.70 | 0.54 | 0.72 | 0.64 | 0.69 | chr19 | chr21 | Y | | Placenta | Lymphocytes | PLAP | |
| | 516b-5p | 155-5p | | | | | | | | | | | | | | | | | |
| Bivariate | hsa-miR- | hsa-miR- | Late | 0.59 | 0.58 | 0.61 | 0.70 | 0.53 | 0.73 | 0.54 | 0.69 | chrX | chr21 | Y | | RBC | Lymphocytes | | |
| | 363-3p | 155-5p | | | | | | | | | | | | | | | | | |
| Univariate | hsa-miR- | | Early | 0.49 | 0.81 | 0.64 | 0.59 | 0.54 | 0.75 | 0.70 | 0.52 | chr19 | | Y | | Placenta | | PLAP | |
| | 516b-5p | | | | | | | | | | | | | | | | | | |
| Univariate | hsa-miR- | | Middle | 0.65 | 0.69 | 0.70 | 0.64 | 0.56 | 0.49 | 0.73 | 0.62 | chr20 | | Y | | Liver | | Input_CD63 | |
| | 941 | | | | | | | | | | | | | | | | | | |

From the foregoing description, it will be apparent that variations and modifications can be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof

All patents and publications mentioned in this specification are herein incorporated by reference to the same extent as if each independent patent and publication was specifically and individually indicated to be incorporated by reference.

1. A panel of isolated nucleic acid biomarkers comprising two or more of the nucleic acid biomarkers listed in Tables 3-11 or 15-18.

2. (canceled)

3. The panel of claim 1, wherein the isolated nucleic acid biomarkers comprise two or more of the isolated nucleic acid biomarkers selected from the group consisting of hsa-miR-423-3p, hsa-miR-516b-5p, hsa-miR-155-5p, hsa-miR-331-3p, hsa-miR-4732-5p, hsa-miR-1273h-3p and hsa-miR-941.

4. The panel of claim 3, wherein the isolated nucleic acid biomarkers comprise:
(a) hsa-miR-331-3p and/or hsa-miR-941;
(b) hsa-miR-423-3p, hsa-miR-516b-5p, hsa-miR-4732-5p, and/or hsa-miR-1273h-3p;
(c) hsa-miR-4732-5p, hsa-miR-516b-5p, and/or hsa-miR-941; or
(d) hsa-miR-155-5p, hsa-miR-331-3p, hsa-miR-1273h-3p, and/or hsa-miR-516b-5p.

5.-8. (canceled)

9. The panel of claim 1, wherein the isolated nucleic acid biomarkers comprise a pair of biomarkers selected from the group consisting of
(a) hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p;
(b) hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p;
(c) hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p;
(d) hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c; or
(e) hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

10.-13. (canceled)

14. A composition of labeled and/or amplified nucleic acid molecules, wherein said labeled and/or amplified nucleic acid molecules correspond to two or more of the nucleic acid biomarkers set forth in Tables 3-11 or 15-18.

15. (canceled)

16. The composition of claim 14, wherein said labeled and/or amplified nucleic acid molecules correspond to two or more of the nucleic acid biomarkers selected from the group consisting of
(a) hsa-miR-423-3p, hsa-miR-516b-5p, hsa-miR-155-5p, hsa-miR-331-3p, hsa-miR-4732-5p, hsa-miR-1273h-3p and hsa-miR-941;
(b) hsa-miR-331-3p and/or hsa-miR-941;
(c) hsa-miR-423-3p, hsa-miR-516b-5p, hsa-miR-4732-5p, and/or hsa-miR-1273h-3p;
(d) hsa-miR-4732-5p, hsa-miR-516b-5p, and/or hsa-miR-941; or
(e) hsa-miR-155-5p, hsa-miR-331-3p, hsa-miR-1273h-3p, and/or hsa-miR-516b-5p.

17.-21. (canceled)

22. The composition of claim 14 wherein said labeled and/or amplified nucleic acid molecules correspond to a pair of biomarkers selected from the group consisting of
(a) hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p;
(b) hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p;
(c) hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p;
(d) hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c; or
(e) hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

23.-26. (canceled)

27. A method for determining a pregnant female's risk of developing placental dysfunction later in the pregnancy comprising:
(a) measuring the amount of two or more of the nucleic acid biomarkers listed in Tables 3-11 or 15-18 in a biological sample obtained from the pregnant female, calculating a risk score based upon the measured amounts of the nucleic acid biomarkers to determine the pregnant female's risk of developing placental dysfunction, and providing a score corresponding to the pregnant female's risk of developing placental dysfunction^{.} or
(b) comprising producing labeled and/or amplified nucleic acid molecules that correspond to two or more of the nucleic acid biomarkers set forth in Tables 3-11 or 15-18 in a biological sample obtained from the pregnant female; measuring the levels of expression of the labeled and/or amplified nucleic acid molecules; calculating a risk score based upon the measured levels of the labeled and/or amplified nucleic acid molecules to determine the pregnant female's risk of developing placental dysfunction.

28.-30. (canceled)

31. The method of claim 27(a), wherein said two or more of the nucleic acid biomarkers are selected from the group consisting of
(a) hsa-miR-423-3p, hsa-miR-516b-5p, hsa-miR-155-5p, hsa-miR-331-3p, hsa-miR-4732-5p, hsa-miR-1273h-3p and hsa-miR-941;
(b) hsa-miR-331-3p and/or hsa-miR-941;
(c) hsa-miR-423-3p, hsa-miR-516b-5p, hsa-miR-4732-5p, and/or hsa-miR-1273h-3p;
(d) hsa-miR-4732-5p, hsa-miR-516b-5p, and/or hsa-miR-941;
(e) hsa-miR-155-5p, hsa-miR-331-3p, hsa-miR-1273h-3p, and/or hsa-miR-516b-5p;
(f) hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p;
(g) hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p;
(h) hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p;
(i) hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c: or
(j) hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

32 -35. (canceled)

36. The method of claim 27(b), wherein said two or more of the nucleic acid biomarkers are selected from the group consisting of
(a) hsa-miR-423-3p, hsa-miR-516b-5p, hsa-miR-155-5p, hsa-miR-331-3p, hsa-miR-4732-5p, hsa-miR-1273h-3p and hsa-miR-941;
(b) hsa-miR-155-5p, hsa-miR-331-3p, hsa-miR-1273h-3p, and/or hsa-miR-516b-5p;
(c) hsa-miR-423-3p, hsa-miR-516b-5p, hsa-miR-4732-5p, and/or hsa-miR-1273h-3p;
(d) hsa-miR-4732-5p, hsa-miR-516b-5p, and/or hsa-miR-941;
(e) hsa-miR-155-5p, hsa-miR-331-3p, hsa-miR-1273h-3p, and/or hsa-miR-516b-5p;
(f) hsa-miR-127-3p/hsa-miR-485-5p, hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-26b-5p/hsa-miR-485-5p, hsa-miR-98-5p/hsa-miR-485-5p, hsa-miR-182-5p/hsa-miR-485-5p, hsa-miR-7-5p/hsa-miR-941, hsa-miR-150-3p/hsa-miR-193b-5p, hsa-miR-378e/hsa-miR-221-5p, hsa-miR-1285-3p/hsa-miR-378c, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p;
(g) hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, and hsa-miR-150-3p/hsa-miR-193b-5p;
(h) hsa-miR-4732-3p/hsa-miR-381-3p, hsa-miR-4732-3p/hsa-miR-941, hsa-miR-155-5p/hsa-miR-3173-5p, hsa-miR-1273h-3p/hsa-miR-3173-5p, hsa-miR-7-5p/hsa-miR-941, hsa-miR-378e/hsa-miR-221-5p, and hsa-miR-345-5p/hsa-miR-324-3p;
(i) hsa-miR-26b-5p/hsa-miR-485-5p, hsa-miR-98-5p/hsa-miR-485-5p, hsa-miR-7-5p/hsa-miR-941, hsa-miR-150-3p/hsa-miR-193b-5p, hsa-miR-378e/hsa-miR-221-5p, and hsa-miR-1285-3p/hsa-mir-378c:
(j) hsa-miR-451a/hsa-miR-155-5p, hsa-miR-125a-5p/hsa-miR-155-5p, hsa-let-7i-5p/hsa-miR-155-5p, hsa-let-7b-5p/hsa-miR-155-5p, hsa-miR-25-3p/hsa-miR-155-5p, hsa-miR-127-3p/hsa-miR-485-5p, hsa-miR-26b-5p/hsa-miR-485-5p, hsa-miR-98-5p/hsa-miR-485-5p, hsa-miR-182-5p/hsa-miR-485-5p, hsa-miR-345-5p/hsa-miR-324-3p, hsa-miR-320b/hsa-miR-155-5p, hsa-miR-181a-5p/hsa-miR-155-5p, hsa-miR-26b-5p/hsa-miR-155-5p, hsa-let-7g-5p/hsa-miR-155-5p, hsa-miR-4443/hsa-miR-155-5p, hsa-miR-425-5p/hsa-miR-155-5p, hsa-miR-146a-5p/hsa-miR-155-5p, hsa-miR-151a-3p/hsa-miR-155-5p, hsa-miR-320a/hsa-miR-155-5p, hsa-miR-30d-5p/hsa-miR-155-5p, hsa-miR-126-3p/hsa-miR-155-5p, hsa-miR-146b-5p/hsa-miR-155-5p, hsa-miR-26a-5p/hsa-miR-155-5p, hsa-miR-625-3p/hsa-miR-155-5p, hsa-miR-423-5p/hsa-miR-155-5p, hsa-miR-99a-5p/hsa-miR-155-5p, hsa-miR-516b-5p/hsa-miR-155-5p, and hsa-miR-363-3p/hsa-miR-155-5p.

37.-50. (canceled)

51. The method of any claim 27, wherein the risk score is calculated based on a ratio of data values.

52. The method of claim 51, wherein data transformation is applied before or after the ratio is determined.
